(19) United States
(12) Patent Application Publication
(10) Pub. No.: US 2013/0178469 A1
(43) Pub. Date: Jul. 11, 2013

(54) NOVEL ANTIVIRAL AGENTS

(71) Applicants: David Ian Rhodes, Heidelberg Heights (AU); John Joseph Deadman, Carlton (AU); Giang Thanh Le, Lower Templestowe (AU); Nicholas Andrew Van de Graff, Prahran (AU); Lu Long, Shanghai (CN); Li Xinming, Shanghai (CN); Feng Xiao, Shanghai (CN); Yu Changjiang, Shanghai (CN)

(72) Inventors: David Ian Rhodes, Heidelberg Heights (AU); John Joseph Deadman, Carlton (AU); Giang Thanh Le, Lower Templestowe (AU); Nicholas Andrew Van de Graff, Prahran (AU); Lu Long, Shanghai (CN); Li Xinming, Shanghai (CN); Feng Xiao, Shanghai (CN); Yu Changjiang, Shanghai (CN)

(73) Assignees: SHANGHAI INSTITUTE OF ORGANIC CHEMISTRY (SIOC), Shanghai (CN); AVEXA LIMITED, Richmond, Victoria (AU)

(21) Appl. No.: 13/807,420

(22) PCT Filed: Jul. 15, 2011
(86) PCT No.: PCT/AU11/00894

§ 371 Date: Mar. 18, 2013

(30) Foreign Application Priority Data
Jul. 16, 2010 (CN) 201010230629.7

(51) Int. Cl. C07D 471/04

(52) U.S. Cl. CPC C07D 471/04
USPC 514/230.8; 544/96; 544/282; 514/252.16; 514/259.41; 544/116; 514/233.2; 544/82; 514/232.5

(57) ABSTRACT

The present invention provides a compound of Formula I or a pharmaceutically acceptable derivative, salt or prodrug thereof. Further provided is a method of treatment or prophylaxis of a viral infection in a subject comprising administering to said subject an effective amount of a compound of Formula I or a pharmaceutically acceptable derivative, salt or prodrug thereof. A pharmaceutical composition or medicament comprising a compound of Formula I is also provided

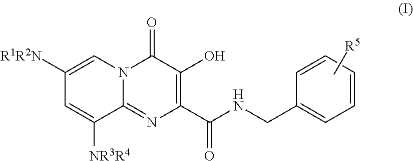

### FIELD OF THE INVENTION

The present invention relates to novel bicyclic pyrimidinone compounds comprising nitrogen-containing substituents at the 7 and 9 positions for the treatment of viral infections, particularly HIV infections.

### BACKGROUND OF THE INVENTION

The retrovirus designated “human immunodeficiency virus” or “HIV” is the etiological agent of a complex disease that progressively destroys the immune system. This disease is known as acquired immune deficiency syndrome or AIDS. As at December 2005 an estimated 40 million people are living with HIV world wide and over 3 million deaths are occurring annually.

A feature of retrovirus replication includes the reverse transcription of the viral genome into proviral DNA and its integration into the host cell genome. These steps are required for HIV replication and are mediated by the virus encoded enzymes, reverse transcriptase and integrase respectively.

HIV infection follows a path of the virus particle binding to cell surface receptors and co-receptors resulting in fusion of the virus particle with the cell. The contents of the virus are released into the cytoplasm where reverse transcription of the HIV genome occurs. Through a series of steps a double stranded proviral DNA copy is produced. The proviral DNA is transported to the nucleus in a complex known as the pre integration complex (PIC) which contains integrase and other viral and possibly cellular proteins. Once inside the nucleus the proviral DNA is integrated into the host cell genome via the action of integrase. Once integrated, transcription and translation of the viral genome can occur resulting in the production of viral proteins and a new viral RNA genome. These proteins and genome assemble at the cell surface and, depending on cell type, possibly other intracellular membranous compartments. Assembled particles then bud out from the cell and during, or soon after, this process mature into infectious HIV particles through the action of the viral protease.

The integration of the proviral genome into the host cell genome requires the action of an integrase which carries out this process in at least three steps, possibly four. The first step involves the assembly of the viral genome into a stable nucleoprotein complex, secondly, processing of two nucleotides from the 3′ termini of the genome to give staggered ends with free 3′ OH residues and thirdly the transfer of these ends into the host cell genome. The final step involves the gap filling and repair of the insertion site in the host genome. There is still some conjecture over whether the integrase performs this final step or whether it is carried out by cellular repair enzymes.

Currently HIV infection can be treated with a number of inhibitors on the market which target reverse transcriptase, protease or entry into the cell. Treatment of HIV infection with these, or a combination of these, drugs is known to be an effective treatment for AIDS and similar diseases. Shortcomings with the current inhibitors include the rapid emergence and increase incidence of resistance and numerous side effects and hence there is a need for new classes of inhibitors targeting proteins such as integrase.

We have previously described compounds for the treatment of viral infections, particularly HIV infections in WO 2008/077188, entitled “Bicyclic Pyrimidinones and Uses Thereof”. We have now found a class of bicyclic pyrimidinones bearing two nitrogen-containing substituents that has not previously been exemplified. The compounds of this class show a significant and surprising advantage in activity over the compounds previously described.

### SUMMARY OF THE INVENTION

In a first aspect, the present invention provides a compound of Formula I or a pharmaceutically acceptable derivative, salt or prodrug thereof wherein:

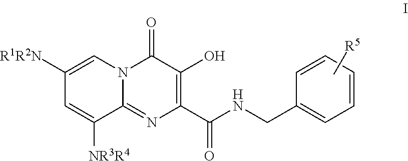

R¹ and R² are each independently selected from the group consisting of hydrogen, C₁₋₄alkyl, C₁₋₄alkylC₃₋₆cycloalkyl, C(O)C₁₋₄alkyl, CO₂C₁₋₄alkyl, —C(O)C(O)NR⁶R⁷, SO₂C₁₋₄alkyl, SO₂NR⁶R⁷; or R¹ and R² taken together with the attached nitrogen form a 4-7 membered heterocyclic ring which contains zero to two additional heteroatoms selected from N, O or S where S can be at the S, S(O) or S(O)₂ oxidation state and wherein said heterocyclic ring is optionally substituted at the carbon or nitrogen atoms with one or more substituents selected from C₁₋₄alkyl, C₃₋₆cycloalkyl, halo, aryl, C(O)C₁₋₄alkyl, SO₂C₁₋₄alkyl, SO₂H, CO₂H, CO₂C₁₋₄alkyl, NR⁶R⁷, C₁₋₄alkylNR⁶R⁷; and further wherein one of the carbon atoms in the heterocyclic ring is optionally a carbonyl carbon;
   wherein R⁶ and R⁷ are independently selected from the group consisting of hydrogen and C₁₋₄alkyl, and C₃₋₆cycloalkyl; or R⁶ and R⁷ taken together with the attached nitrogen form a 4-7 membered heterocyclic ring which contains zero to two additional heteroatoms selected from N and O;
R³ and R⁴ are each independently selected from the group consisting of hydrogen, C₁₋₄alkyl, C₁₋₄alkylC₃₋₆cycloalkyl, C(O)C₁₋₄alkyl, CO₂C₁₋₄alkyl, —C(O)C(O)NR⁸R⁹, SO₂C₁₋₄alkyl, SO₂NR⁸R⁹; or R³ and R⁴ taken together with the attached nitrogen form a 4-7 membered heterocyclic ring which contains zero to two additional heteroatoms selected from N, O or S where S can be at the S, S(O) or S(O)₂ oxidation state and wherein said heterocyclic ring is optionally substituted at the carbon or nitrogen atoms with one or more substituents selected from C₁₋₄alkyl, C₃₋₆cycloalkyl, halo, aryl, C(O)C₁₋₄alkyl, SO₂C₁₋₄alkyl, SO₂H, CO₂H, CO₂C₁₋₄alkyl, NR⁸R⁹; C₁₋₄alkylNR⁸R⁹; and further wherein one of the carbon atoms in the heterocyclic ring is optionally a carbonyl carbon;
   wherein R⁸ and R⁹ are each independently selected from the group consisting of hydrogen, C₁₋₄alkyl and C₃₋₆cycloalkyl; or R⁸ and R⁹ taken together with the attached nitrogen form a 4-7 membered heterocyclic ring which contains zero to two additional heteroatoms selected from N and O;
R⁵ is 0-3 substituents each of which is independently selected from the group consisting of halo, C₁₋₁₀alkyl, C₂₋₁₀alkenyl, —O—C₁₋₁₀alkyl, C(O)C₁₋₄alkyl CO₂H, CO₂C₁₋₄alkyl, CN, NH₂, NO₂, CF₃, aryl, heteroaryl, alkylaryl, alkylheteroaryl, —O-alkylaryl.

The present inventors have further found that bicyclic pyrimidinones of Formula I in which the 9 position is substituted by a sulphonamide or cyclic sulphonamide show enhanced activity against the HIV virus. Accordingly, in a second aspect, the present invention provides a compound of Formula I or a pharmaceutically acceptable derivative, salt or prodrug thereof:

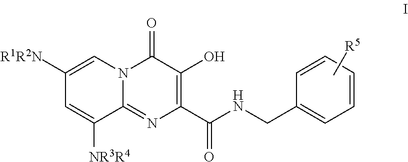

wherein R¹ and R² are each independently selected from the group consisting of hydrogen, C₁₋₄alkyl, C₁₋₄alkylC₃₋₆cycloalkyl, C(O)C₁₋₄alkyl, CO₂C₁₋₄alkyl, —C(O)C(O)NR⁸R⁹, SO₂C₁₋₄alkyl, SO₂NR⁸R⁹; or R¹ and R² taken together with the attached nitrogen form a 4-7 membered heterocyclic ring which contains zero to two additional heteroatoms selected from N, O or S where S can be at the S, S(O) or S(O)₂ oxidation state and wherein said heterocyclic ring is optionally substituted at the carbon or nitrogen atoms with one or more substituents selected from C₁₋₄alkyl, C₃₋₆cycloalkyl, halo, aryl, C(O)C₁₋₄alkyl, SO₂C₁₋₄alkyl, SO₂H, CO₂H, CO₂C₁₋₄alkyl, NR⁶R⁷, C₁₋₄alkylNR⁶R⁷; and further wherein one of the carbon atoms in the heterocyclic ring is optionally a carbonyl carbon;
   wherein R⁶ and R⁷ are independently selected from the group consisting of hydrogen and C₁₋₄alkyl, and C₃₋₆cycloalkyl; or R⁶ and R⁷ taken together with the attached nitrogen form a 4-7 membered heterocyclic ring which contains zero to two additional heteroatoms selected from N and O;
wherein R³ is C₁₋₄alkyl and R⁴ is SO₂C₁₋₄alkyl;
or wherein NR³R⁴ forms a cyclic sulphonamide of the formula II:

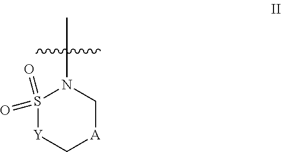

wherein Y is selected from the group consisting of a bond, CH₂, NH and NC₁₋₄alkyl; and A is a bond or CH₂;
wherein R⁵ is 0-3 substituents each of which is independently selected from the group consisting of halo, C₁₋₁₀alkyl, C₂₋₁₀alkenyl, —O—C₁₋₁₀alkyl, C(O)C₁₋₄alkyl CO₂H, CO₂C₁₋₄alkyl, CN, NH₂, NO₂, CF₃, aryl, heteroaryl, alkylaryl, alkylheteroaryl, —O-alkylaryl.

In a third aspect, the present invention provides a method of treatment or prophylaxis of a viral infection in a subject comprising administering to said subject an effective amount of a compound of the present invention or a pharmaceutically acceptable derivative, salt or prodrug thereof.

In a fourth aspect, the present invention provides the use of a compound of the present invention or a pharmaceutically acceptable derivative, salt or prodrug thereof in the preparation of a medicament for the treatment or prophylaxis of a viral infection in a subject.

In a fifth aspect, the present invention provides a pharmaceutical composition comprising a compound of the present invention or a pharmaceutically acceptable derivative, salt or prodrug thereof and a pharmaceutically acceptable carrier, diluent or excipient.

### DETAILED DESCRIPTION OF THE INVENTION

In the specification below, where we refer to positions 7 and 9, this refers to the following sites on the compound of formula I:

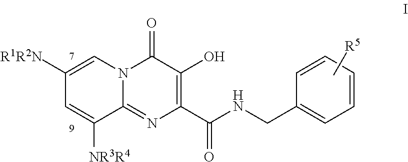

In a first aspect the present invention provides a compound of Formula I or a pharmaceutically acceptable derivative, salt or prodrug thereof wherein:

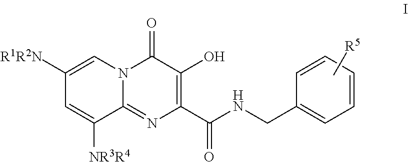

R¹ and R² are each independently selected from the group consisting of hydrogen, C₁₋₄alkyl, C₁₋₄alkylC₃₋₆cycloalkyl, C(O)C₁₋₄alkyl, CO₂C₁₋₄alkyl, —C(O)C(O)NR⁶R⁷, SO₂C₁₋₄alkyl, SO₂NR⁶R⁷; or R¹ and R² taken together with the attached nitrogen form a 4-7 membered heterocyclic ring which contains zero to two additional heteroatoms selected from N, O or S where S can be at the S, S(O) or S(O)₂ oxidation state and wherein said heterocyclic ring is optionally substituted at the carbon or nitrogen atoms with one or more substituents selected from C₁₋₄alkyl, C₃₋₆cycloalkyl, halo, aryl, C(O)C₁₋₄alkyl, SO₂C₁₋₄alkyl, SO₂H, CO₂H, CO₂C₁₋₄alkyl, NR⁶R⁷, C₁₋₄alkylNR⁶R⁷; and further wherein one of the carbon atoms in the heterocyclic ring is optionally a carbonyl carbon;
wherein R⁶ and R⁷ are independently selected from the group consisting of hydrogen and C₁₋₄alkyl, and C₃₋₆cycloalkyl; or R⁶ and R⁷ taken together with the attached nitrogen form a 4-7 membered heterocyclic ring which contains zero to two additional heteroatoms selected from N and O;

R³ and R⁴ are each independently selected from the group consisting of hydrogen, C₁₋₄alkyl, C₁₋₄alkylC₃₋₆cycloalkyl, C(O)C₁₋₄alkyl, CO₂C₁₋₄alkyl, —C(O)C(O)NR⁸R⁹, SO₂C₁₋₄alkyl, SO₂NR⁸R⁹; or R³ and R⁴ taken together with the attached nitrogen form a 4-7 membered heterocyclic ring which contains zero to two additional heteroatoms selected from N, O or S where S can be at the S, S(O) or S(O)₂ oxidation state and wherein said heterocyclic ring is optionally substituted at the carbon or nitrogen atoms with one or more substituents selected from C₁₋₄alkyl, C₃₋₆cycloalkyl, halo, aryl, C(O)C₁₋₄alkyl, SO₂C₁₋₄alkyl, SO₂H, CO₂H, CO₂C₁₋₄alkyl, NR⁸R⁹; C₁₋₄alkylNR⁸R⁹; and further wherein one of the carbon atoms in the heterocyclic ring is optionally a carbonyl carbon;
wherein R⁸ and R⁹ are each independently selected from the group consisting of hydrogen, C₁₋₄alkyl and C₃₋₆cycloalkyl; or R⁸ and R⁹ taken together with the attached nitrogen form a 4-7 membered heterocyclic ring which contains zero to two additional heteroatoms selected from N and O;

R⁵ is 0-3 substituents each of which is independently selected from the group consisting of halo, C₁₋₁₀alkyl, C₂₋₁₀alkenyl, —O—C₁₋₁₀alkyl, C(O)C₁₋₄alkyl CO₂H, CO₂C₁₋₄alkyl, CN, NH₂, NO₂, CF₃, aryl, heteroaryl, alkylaryl, alkylheteroaryl, —O-alkylaryl.

In one embodiment, when R³ and R⁴ are taken together with the attached nitrogen form a 4-7 membered heterocyclic ring and further wherein one of the carbon atoms in the heterocyclic ring is a carbonyl carbon, the carbonyl carbon is adjacent to the attached nitrogen.

In one embodiment, R³ and R⁴ taken together with the attached nitrogen form a 4-7 membered heterocyclic ring which contains zero to two additional heteroatoms selected from N, O or S where S can be at the S, S(O) or S(O)₂ oxidation state and wherein said heterocyclic ring is optionally substituted at the carbon or nitrogen atoms with one or more substituents selected from C₁₋₄alkyl; and further wherein one of the carbon atoms in the heterocyclic ring is optionally a carbonyl carbon.

In one embodiment, when R³ and R⁴ are taken together with the attached nitrogen form a 5-7 membered heterocyclic ring.

The present inventors have further found that bicyclic pyrimidinones of Formula I in which the 9 position is substituted by a sulphonamide or cyclic sulphonamide show enhanced activity against the HIV virus. This is demonstrated in Tables 1 to 5 below which show biological activities for certain compounds of the present invention. Accordingly, in a preferred embodiment, R³ and R⁴ are taken together with the attached nitrogen forms a 4-7 membered heterocyclic ring which contains at least one additional sulfur heteroatom in the S(O)₂ oxidation state adjacent to the attached nitrogen, and wherein the ring contains one additional nitrogen atom, wherein the additional nitrogen atom is optionally substituted with C₁₋₄alkyl, preferably with methyl.

Further, in a second aspect, the present invention provides a compound of Formula I or a pharmaceutically acceptable derivative, salt or prodrug thereof:

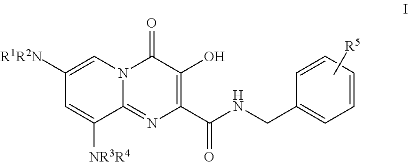

wherein R¹ and R² are each independently selected from the group consisting of hydrogen, C₁₋₄alkyl, C₁₋₄alkylC₃₋₆cycloalkyl, C(O)C₁₋₄alkyl, CO₂C₁₋₄alkyl, —C(O)C(O)NR⁶R⁷, SO₂C₁₋₄alkyl, SO₂NR⁶R⁷; or R¹ and R² taken together with the attached nitrogen form a 4-7 membered heterocyclic ring which contains zero to two additional heteroatoms selected from N, O or S where S can be at the S, S(O) or S(O)₂ oxidation state and wherein said heterocyclic ring is optionally substituted at the carbon or nitrogen atoms with one or more substituents selected from C₁₋₄alkyl, C₃₋₆cycloalkyl, halo, aryl, C(O)C₁₋₄alkyl, SO₂C₁₋₄alkyl, SO₂H, CO₂H, CO₂C₁₋₄alkyl, NR⁶R⁷C₁₋₄alkylNR⁶R⁷; and further wherein one of the carbon atoms in the heterocyclic ring is optionally a carbonyl carbon;
wherein R⁶ and R⁷ are independently selected from the group consisting of hydrogen and C₁₋₄alkyl, and C₃₋₆cycloalkyl; or R⁶ and R⁷ taken together with the attached nitrogen form a 4-7 membered heterocyclic ring which contains zero to two additional heteroatoms selected from N and O;
wherein R³ is C₁₋₄alkyl and R⁴ is SO₂C₁₋₄alkyl;
or wherein NR³R⁴ forms a cyclic sulphonamide of the formula II:

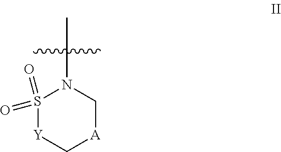

wherein Y is selected from the group consisting of a bond, CH₂, NH and NC₁₋₄alkyl; and A is a bond or CH₂;
wherein R⁵ is 0-3 substituents each of which is independently selected from the group consisting of halo, C₁₋₁₀alkyl, C₂₋₁₀alkenyl, —O—C₁₋₁₀alkyl, C(O)C₁₋₄alkyl CO₂H, CO₂C₁₋₄alkyl, CN, NH₂, NO₂, CF₃, aryl, heteroaryl, alkylaryl, alkylheteroaryl, —O-alkylaryl.

In one embodiment of the first and second aspects, when R¹ and R² are taken together with the attached nitrogen form a 4-7 membered heterocyclic ring and further wherein one of the carbon atoms in the heterocyclic ring is a carbonyl carbon, the carbonyl carbon is adjacent to the attached nitrogen.

In one embodiment of the first and second aspects, R¹ and R² taken together with the attached nitrogen form a 4-7 membered heterocyclic ring which contains zero to two additional heteroatoms selected from N or O, wherein said heterocyclic ring is optionally substituted at the carbon or nitrogen atoms with one or more C₁₋₄alkyl substituents; and further wherein one of the carbon atoms in the heterocyclic ring is optionally a carbonyl carbon.

In one embodiment of the first and second aspects, when R¹ and R² are taken together with the attached nitrogen form a 5-7 membered heterocyclic ring.

In one embodiment of the first and second aspects, R¹ and R² taken together with the attached nitrogen form morpholine.

In one embodiment of the first and second aspects, R¹ and R² taken together with the attached nitrogen form piperazine

In one embodiment of the first and second aspects, R¹ and R² taken together with the attached nitrogen form N-methyl piperazine.

Preferably R⁵ is 1-3 substituents, more preferably R⁵ is 1-2 substituents, even more preferably R⁵ is 1 or 2 substituents.

In one embodiment of the first and second aspects, R⁵ is 1-2 substituents each independently selected from halo. Preferably, R⁵ is 1-2 substituents each independently selected from Cl or F. In one embodiment, R⁵ is a fluorine substituent at the 4-position the 3 and 4-positions of the phenyl ring.

In another embodiment of the second aspect, R³ is C₁₋₄alkyl and R⁴ is SO₂C₁₋₄alkyl.

In one embodiment of the second aspect, NR³R⁴ forms a cyclic sulphonamide of the formula II:

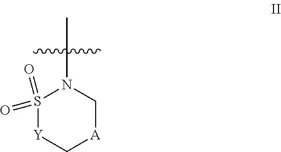

wherein Y is selected from the group consisting of a bond, CH₂, NH and NC₁₋₄alkyl; and A is a bond or CH₂.

As understood by a person skilled in the art when A and Y are each a bond the cyclic sulphonamide is a 4 membered ring.

As understood by a person skilled in the art when A is a bond and Y is not a bond, or vice versa, the cyclic sulphonamide is a 5 membered ring.

As understood by a person skilled in the art when A is CH₂ and Y is not a bond, the cyclic sulphonamide is a 6 membered ring.

In a preferred embodiment of the first and second aspects, NR¹R² is selected from the group consisting of:

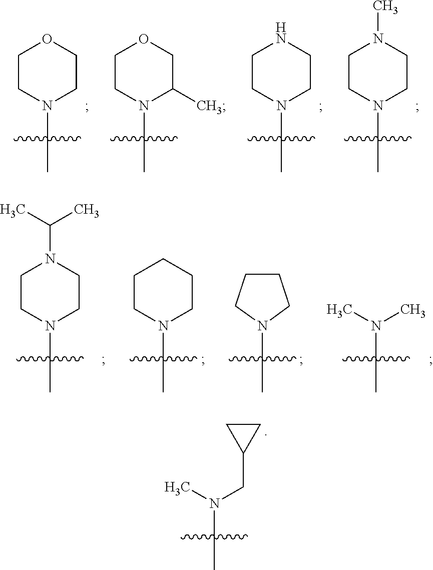

In a more preferred embodiment of the first and second aspects, NR¹R² is selected from the group consisting of:

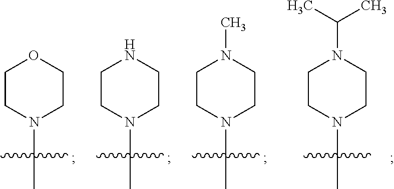

even more preferably NR¹R² is selected from the group consisting of:

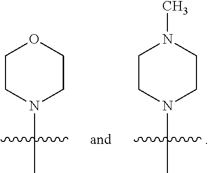

In a preferred embodiment of the first aspect, NR³R⁴ is selected from the group consisting of:

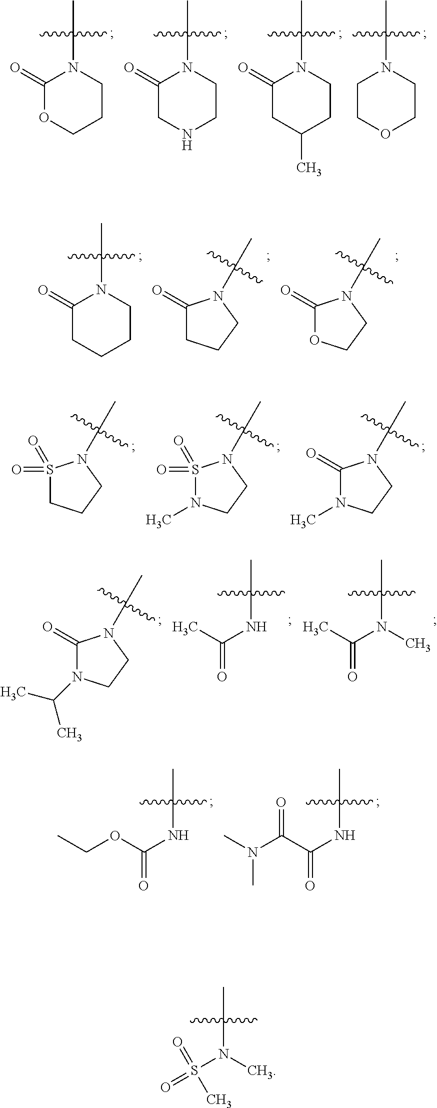

In a more preferred embodiment of the first aspect, NR³R⁴ is selected from the group consisting of:

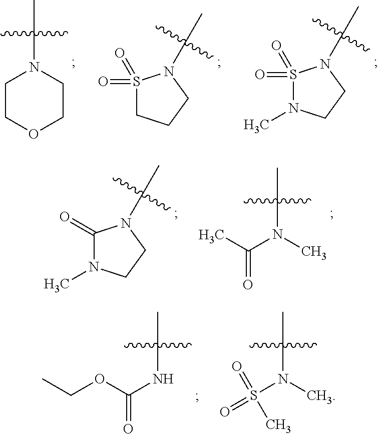

In a preferred embodiment of the second aspect, NR³R⁴ forms a cyclic sulphonamide of the formula II:

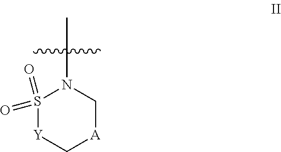

wherein Y is selected from the group consisting of a bond, CH₂, NH and NC₁₋₄alkyl; and A is a bond or CH₂.

In another preferred embodiment of the second aspect, NR³R⁴ is selected from the group consisting of:

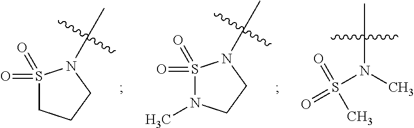

more preferably NR³R⁴ is:

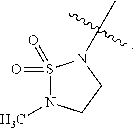 [SMILES: *N1CCN(C)S1(=O)=O]

In a preferred embodiment the present invention comprises a compound of formula I selected from the group consisting of:

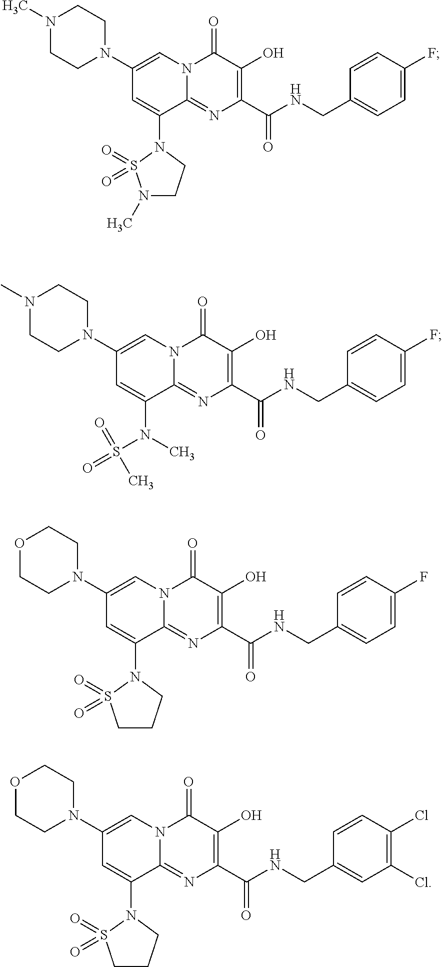 [SMILES: CN1CCN(c2cc(N3CCN(C)S3(=O)=O)c3nc(C(=O)NCc4ccc(F)cc4)c(O)c(=O)n3c2)CC1 ; CN1CCN(c2cc(N(C)S(C)(=O)=O)c3nc(C(=O)NCc4ccc(F)cc4)c(O)c(=O)n3c2)CC1 ; O=C(NCc1ccc(F)cc1)c1nc2c(N3CCCS3(=O)=O)cc(N3CCOCC3)cn2c(=O)c1O ; O=C(NCc1ccc(Cl)c(Cl)c1)c1nc2c(N3CCCS3(=O)=O)cc(N3CCOCC3)cn2c(=O)c1O]

As understood by a person skilled in the art 4-7 membered heterocyclic ring means a heterocyclic ring with 4, 5, 6 or 7 atoms or a range comprising any of two of those integers.

As understood by a person skilled in the art 5-7 membered heterocyclic ring means a heterocyclic ring with 5, 6 or 7 atoms or a range comprising any of two of those integers.

As understood by a person skilled in the art a heterocyclic ring which contains zero to two additional heteroatoms means a heterocyclic ring that contains zero, one or two additional heteroatoms or a range comprising any of two of those integers.

As used herein, the term “halo” or “halogen” refers to fluorine (fluoro), chlorine (chloro), bromine (bromo) or iodine (iodo).

As used herein, the term “alkyl” either used alone or in compound terms such as NH(alkyl) or N(alkyl)₂, refers to monovalent straight chain or branched hydrocarbon groups, having 1 to 3 (meaning 1, 2 or 3 carbon atoms or a range comprising any of two of those integers), 1 to 6 (meaning 1, 2, 3, 4, 5 or 6 carbon atoms or a range comprising any of two of those integers), or 1 to 10 carbon atoms (meaning 1, 2, 3, 4, 5, 6, 7, 8, 9 or 10 carbon atoms or a range comprising any of two of those integers) as appropriate. For example, suitable alkyl groups include, but are not limited to methyl, ethyl, propyl, isopropyl, n-butyl, sec-butyl, tert-butyl, pentyl, 2-methylbutyl, 3-methylbutyl, n-hexyl, 2-, 3- or 4-methylpentyl, 2-ethylbutyl, n-hexyl or 2-, 3-, 4- or 5-methylpentyl.

As understood by a person skilled in the art, the term “C₁₋₄alkyl” means an alkyl chain with 1, 2, 3 or 4 carbon atoms or a range comprising any of two of those integers.

As understood by a person skilled in the art, the term “C₁₋₁₀alkyl” means an alkyl chain with 1, 2, 3, 4, 5, 6, 7, 8, 9 or 10 carbon atoms or a range comprising any of two of those integers.

As used herein, the term “alkenyl” refers to a straight chain or branched hydrocarbon groups having one or more double bonds between carbon atoms. Suitable alkenyl groups include, but are not limited to, ethenyl, allyl, propenyl, iso-propenyl, butenyl, pentenyl and hexenyl.

As understood by a person skilled in the art, the term “C₂₋₁₀alkenyl” means an alkenyl chain with 2, 3, 4, 5, 6, 7, 8, 9 or 10 carbon atoms or a range comprising any of two of those integers.

The term “cycloalkyl” as used herein, refers to cyclic hydrocarbon groups. Suitable cycloalkyl groups include, but are not limited to cyclopropyl, cyclobutyl, cyclopentyl and cyclohexyl.

As understood by a person skilled in the art, the term “C₃₋₆cycloalkyl” means a cycloalkyl group with 3, 4, 5, or 6 carbon atoms or a range comprising any of two of those integers.

The term “aryl” as used herein, refers to a C₆-C₁₀ aromatic hydrocarbon group, for example phenyl or naphthyl.

The term “alkylaryl” includes, for example, benzyl.

The term “heterocycle” when used alone or in compound words includes monocyclic, polycyclic, fused or conjugated hydrocarbon residues, preferably C₃₋₆ (which means 3, 4, 5, or 6 carbon atoms or a range comprising any of two of those integers), wherein one or more carbon atoms (and where appropriate, hydrogen atoms attached thereto) are replaced by a heteroatom so as to provide a non-aromatic residue. The bonds between atoms may be saturated or unsaturated. Suitable heteroatoms include, O, N and S. Where two or more carbon atoms are replaced, this may be by two or more of the same heteroatom or by different heteroatoms. Suitable examples of heterocyclic groups may include pyrrolidinyl, piperidyl, piperazinyl, morpholino, quinolinyl, isoquinolinyl, thiomorpholino, dioxanyl, 2,2′-dimethyl-[1,3]-dioxolanyl, tetrahydrofuranyl, tetrahydropyranyl, tetrahydropyrrolyl, cyclic sulfonamides such as sultams etc.

The term “sultam” is directed to cyclic sulfonamides in which the nitrogen and sulfur atoms of the sulfonamide group form part of the heterocyclic ring.

Preferred sultams include:

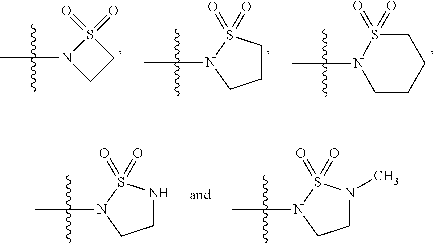

The term “heteroaryl” includes a 5- or 6-membered heteroaromatic ring containing one or more heteroatoms selected from O, N and S. Suitable examples of heteroaryl groups include furanyl, thiophenyl, tetrazolyl, 1,2,3-triazolyl, 1,2,4-triazolyl, imidazolyl, pyrazolyl, pyridinyl, pyrimidinyl, oxazolyl, oxadiazolyl, thioazolyl, thiodiazolyl etc. The heteroaromatic ring may be fused to a 5- or 6-membered aromatic or heteroaromatic ring to form a bicyclic aromatic ring system eg benzofuran.

Unless otherwise stated, each alkyl, cycloalkyl, alkylaryl, aryl, heterocyclyl, or heteroaryl group may be optionally substituted with one or more of C₁-C₃alkyl, C₃-C₆cycloalkyl, C₆aryl, heterocyclyl, heteroaryl, C₁-C₃alkylOH, alkylaryl, OH, OC₁-C₃alkyl, halo, CN, NO₂, CO₂H, CO₂C₁-C₃alkyl, CONH₂, CONH(C₁-C₃alkyl), CON(C₁-C₃alkyl)₂, trifluoromethyl, NH₂, NH(C₁-C₃alkyl) or N(C₁-C₃alkyl)₂. For example, an optionally substituted aryl group may be 4-methylphenyl or 4-hydroxyphenyl group, and an optionally substituted alkyl group may be 2-hydroxyethyl, trifluoromethyl, or difluoromethyl. Each optional alkyl, cycloalkyl, alkylaryl, aryl, heterocyclyl, or heteroaryl substituent may also be optionally substituted.

“C₁-C₃alkyl” means an alkyl chain with 1, 2 or 3 carbon atoms or a range comprising any of two of those integers.

As understood by a person skilled in the art “0-3 substituents” means 0, 1, 2 or 3 substituents or a range comprising any two of those integers.

Examples of optional substituents also include suitable nitrogen protecting groups (see “Protective Groups in Organic Synthesis” Theodora Greene and Peter Wuts, third edition, Wiley Interscience, 1999).

The salts of the compound of formula I are preferably pharmaceutically acceptable, but it will be appreciated that non-pharmaceutically acceptable salts also fall within the scope of the present invention, since these are useful as intermediates in the preparation of pharmaceutically acceptable salts.

The term “pharmaceutically acceptable derivative” may include any pharmaceutically acceptable salt, hydrate or prodrug, or any other compound which upon administration to a subject, is capable of providing (directly or indirectly) a compound of formula I or an antibacterially active metabolite or residue thereof.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1^{st} edition, 2002, Wiley-VCH.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

Hydroxyl groups may be esterified with groups including lower alkyl carboxylic acids, such as acetic acid and 2,2-dimethylpropionic acid, or sulfonated with groups including alkyl sulfonic acids, such as methyl sulfonic acid.

This invention also encompasses pharmaceutical compositions containing prodrugs of compounds of formula I. This invention also encompasses methods of treating or preventing a viral infection in a subject by administering prodrugs of compounds of the formula I. Compounds of formula I having free amino, amido, hydroxy or carboxylic groups can be converted into prodrugs.

Prodrugs include compounds wherein an amino acid residue, or a polypeptide chain of two or more (eg, two, three or four) amino acid residues which are covalently joined to free amino, hydroxy and carboxylic acid groups of compounds of formula I. The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of formula I through the carbonyl carbon prodrug sidechain. Prodrugs also include phosphate derivatives of compounds of formula I (such as acids, salts of acids, or esters) joined through a phosphorus-oxygen bond to a free hydroxyl of compounds of formula I.

It will also be recognised that the compounds of formula I may possess asymmetric centres and are therefore capable of existing in more than one stereoisomeric form.

The invention thus also relates to compounds in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution.

The present invention provides a method of treatment or prophylaxis of a viral infection in a subject comprising administering to said subject an effective amount of a compound of the present invention or a pharmaceutically acceptable derivative, salt or prodrug thereof.

The present invention provides the use of a compound of the present invention or a pharmaceutically acceptable derivative, salt or prodrug thereof, in the preparation of a medicament for the treatment or prophylaxis of a viral infection in a subject.

Preferably, the viral infection is a HIV or SIV infection.

The present invention provides a pharmaceutical composition comprising a compound of the present invention or a pharmaceutically acceptable derivative, salt or prodrug thereof, and a pharmaceutically acceptable carrier, diluent or excipient.

The compositions of the present invention may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The compounds of the present invention may be administered by any suitable means, for example, parenterally, such as by subcutaneous, intravenous, intramuscular, or intracisternal injection or infusion techniques (e.g., as sterile injectable aqueous or non-aqueous solutions or suspensions).

Pharmaceutical formulations include those for oral, rectal, nasal, topical (including buccal and sub-lingual), vaginal or parenteral (including intramuscular, sub-cutaneous and intravenous) administration or in a form suitable for administration by inhalation or insufflation. The compounds of the invention, together with a conventional adjuvant, carrier or diluent, may thus be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, in the form of suppositories for rectal administration; or in the form of sterile injectable solutions for parenteral (including subcutaneous) use.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The subjects treated in the above method are mammals, including, but not limited to, cows, sheep, goats, horses, dogs, cats, guinea pigs, rats or other bovine, ovine, equine, canine, feline, rodent or murine species, and preferably a human being, male or female.

The term “effective amount” means the amount of the subject composition that will elicit the biological or medical response of a tissue, system, animal or human that is being sought by the researcher, veterinarian, medical doctor or other clinician.

As would be understood by those skilled in the art of treating viral infections, and particularly HIV infections, the term “treatment” does not necessarily mean that the viral infection is completely cured. The term “treatment” encompasses any reduction in the viral load and/or inhibition of replication in the subject being treated.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The pharmaceutical composition and method of the present invention may further comprise other therapeutically active compounds which are usually applied in the treatment of the above mentioned pathological conditions. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

When other therapeutic agents are employed in combination with the compounds of the present invention they may be used for example in amounts as noted in the Physician Desk Reference (PDR) or as otherwise determined by one of ordinary skill in the art.

In the treatment or prevention of conditions which require HIV inhibition or HIV integrase enzyme inhibition an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In order that the nature of the present invention may be more clearly understood preferred forms thereof will now be described by reference to the following non-limiting Examples.

### EXAMPLES

### Methods

### HPLC Conditions

All HPLC measurements were performed on a Waters 2690 Alliance System.

### Method 1

Column:

Waters Exterra C18 Column (Part #186000410) at 30° C., flow rate 0.4 mL/min, spectra measured at 254 nM

### Buffers:

Buffer A: 100% water, Buffer B: 100% acetonitrile, Buffer C: 2% aqueous TFA Gradient: (linear gradient curve 6)

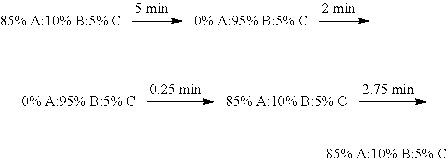

### Method 2

### Column:

Merck C18 Chromolith Column (Part #1.02129.0001) at 30° C., flow rate 4 mL/min, spectra measured at 254 nM

### Buffers:

Buffer A: 100% water, Buffer B: 100% acetonitrile, Buffer C: 2% aqueous TFA

Gradient: (linear gradient curve 6)

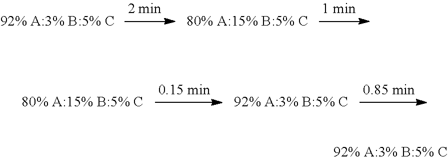

### Method 3

### Column:

Merck C18 Chromolith Column (Part #1.02129.0001) at 30° C., flow rate 4 mL/min, spectra measured at 254 nM

### Buffers:

Buffer A: 100% water, Buffer B: 100% acetonitrile, Buffer C: 2% aqueous TFA

Gradient: (linear gradient curve 6)

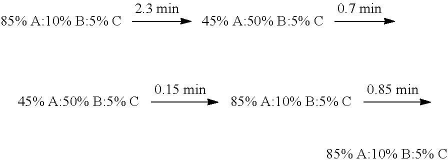

### Method 4

### Column:

Merck C18 Chromolith Column (Part #1.02129.0001) at 30° C., flow rate 4 mL/min, spectra measured at 254 nM Buffers.

Buffer A: 100% water, Buffer B: 100% acetonitrile, Buffer C: 2% aqueous TFA

Gradient: (linear gradient curve 6)

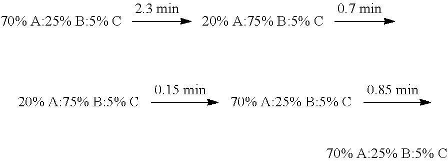

### Method 5

### Column:

Phenomenex Gemini C18 Column (Part #344382-3) at 30° C., flow rate 0.4 mL/min, spectra measured at 254 nM

### Buffers:

Buffer A: 100% water, Buffer B: 100% acetonitrile, Buffer C: 2% aqueous TFA

Gradient: (linear gradient curve 6)

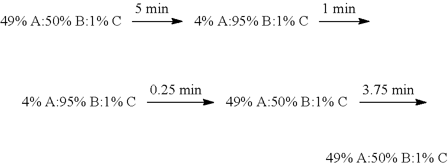

### Method 6

### Column:

Phenomenex Gemini C18 Column (Part #344382-3) at 30° C., flow rate 0.4 mL/min, spectra measured at 254 nM

### Buffers:

Buffer A: 100% water, Buffer B: 100% acetonitrile, Buffer C: 2% aqueous TFA

Gradient: (linear gradient curve 6)

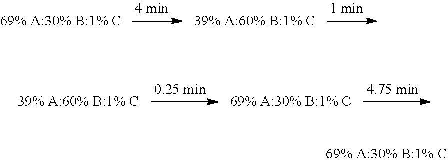

### Method 7

### Column:

Waters Symmetry® C18 Column (Part No WAT045905) at 25° C., flow rate 1 mL/min, spectra measured at 254 nM

### Buffers:

Buffer A: 100% acetonitrile, Buffer B: 0.1% aqueous TFA

Gradient: (linear gradient curve 6)

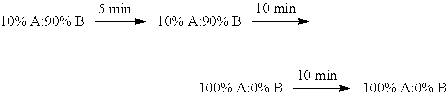

### SYNTHETIC EXAMPLES

### Synthesis of Starting Material—Synthetic Scheme 1

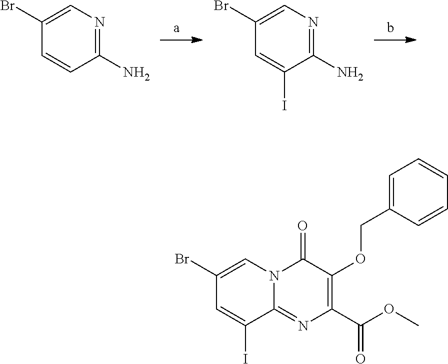 [SMILES: Nc1ccc(Br)cn1 ; Nc1ncc(Br)cc1I ; COC(=O)c1nc2c(I)cc(Br)cn2c(=O)c1OCc1ccccc1]

Reagents and conditions (a) I₂, HIO₄.H₂O, AcOH, H₂SO₄.H₂O, 75° C., 7 h, 70% (b) i) DAF, pTSOH, 100° C., 30 h; ii) BnBr, K₂CO₃, DMF, 70° C., 16 h, 16% 2-steps.

### Synthesis of the Starting Material 3-benzyloxy-7-dimethylamino-9-iodo-4-oxo-4H-pyrido[1,2-a]pyrimidine-2-carboxylic Acid Methyl Ester

### Step 1: Preparation of Preparation of 3-iodo-N⁵,N⁵-dimethylpyridine-2,5-diamine

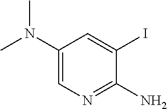

Paraformaldehyde (1 g, 33.3 mmol) was suspended in MeOH (30 ml) and refluxed for 2 h, then cooled to room temperature. Then 3-iodopyridine-2,5-diamine (2 g, 8.5 mmol) was added to the above mixture, followed by NaCNBH₃ (4.7 g, 76 mmol) in small portions. After TLC plate indicated that the reaction was over, most of the solvent was removed under reduced pressure. Water was added and the mixture was extracted with ethyl acetate. The combined organic layers were washed with water, dried and concentrated in vacuo. The residue was purified by column chromatography to give the desired product (0.8 g, 35% yield).

¹H NMR (300 MHz, DMSO-d⁶) δ 2.72 (s, 6H), 5.32 (s, 2H), 7.48 (d, J=2.6 Hz, 1H), 7.62 (d, J=2.6 Hz, 1H)

MS (ESI⁺) m/z 264 (M+1)

### Step 2: Preparation of 3-Acetoxy-7-dimethylamino-9-iodo-4-oxo-4H-pyrido[1,2-a]pyrimidine-2-carboxylic acid methyl ester

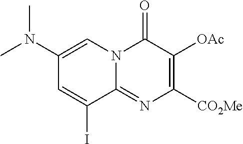 [SMILES: COC(=O)c1nc2c(I)cc(N(C)C)cn2c(=O)c1OC(C)=O]

The product of example 227 (100 mg, 0.38 mmol), p-toluenesulfonic acid (10 mg, 0.52 mmol), DAF (400 mg, 1.53 mmol) were mixed in MeOH (0.5 ml). The mixture was stirred at 80° C. for 8 h, after which the solvent was evaporated and acetic anhydride (400 mg, 4 mmol) in pyridine (5 ml) was added. The mixture was refluxed for 1 h. After cooling to the room temperature, the mixture was concentrated under reduced pressure. The resulting residue was purified by column chromatography to give the desired product (20 mg, 12% yield).

¹H NMR (300 MHz, DMSO-d⁶) δ 2.31 (s, 3H), 3.03 (s, 6H), 3.90 (s, 3H), 8.00 (d, J=2.4 Hz, 1H), 8.56 (d, J=2.7 Hz, 1H)

MS (ESI⁺) m/z 454 (M+23)

### Step 3: Preparation of methyl 7-(dimethylamino)-3-hydroxy-9-iodo-4-oxo-4H-pyrido[1,2-a]pyrimidine-2-carboxylate

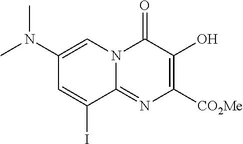 [SMILES: COC(=O)c1nc2c(I)cc(N(C)C)cn2c(=O)c1O]

To a solution of the product of example 228 (15 mg, 0.035 mmol) in MeOH (5 ml) was added K₂CO₃ (30 mg, 0.217 mmol). The mixture was refluxed for 5 h and then extracted with dichloromethane and water. Organic layer was concentrated into dryness to give the titled product (12 mg, yield 80%).

¹H NMR (300 MHz, DMSO-d⁶) δ 2.97 (s, 6H), 3.89 (s, 3H), 7.84 (d, J=2.3 Hz, 1H), 8.35 (d, J=2.5 Hz, 1H), 10.18 (s, 1H)

### Step 4: Preparation of 3-Benzyloxy-7-dimethylamino-9-iodo-4-oxo-4H-pyrido[1,2-a]pyrimidine-2-carboxylic acid methyl ester

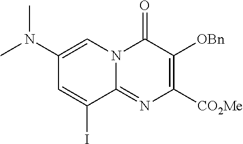 [SMILES: COC(=O)c1nc2c(I)cc(N(C)C)cn2c(=O)c1OCc1ccccc1]

The title compound was prepared by adapting methods described in example 8.1-8.2 of International Patent Application Publication No. WO2008/077188 to Avexa Limited using the product of example 65.

¹H NMR (300 MHz, DMSO-d⁶) δ 3.02 (s, 6H), 3.84 (s, 3H), 5.17 (s, 2H), 7.28-7.48 (m, 5H), 8.05 (d, J=2.7 Hz, 1H), 8.49 (d, J=2.7 Hz, 1H)

### Example 1

### Preparation of N-(4-fluorobenzyl)-3-hydroxy-7-(4-methylpiperazin-1-yl)-4-oxo-9-(2-oxo-1,3-oxazinan-3-yl)-4H-pyrido[1,2-a]pyrimidine-2-carboxamide

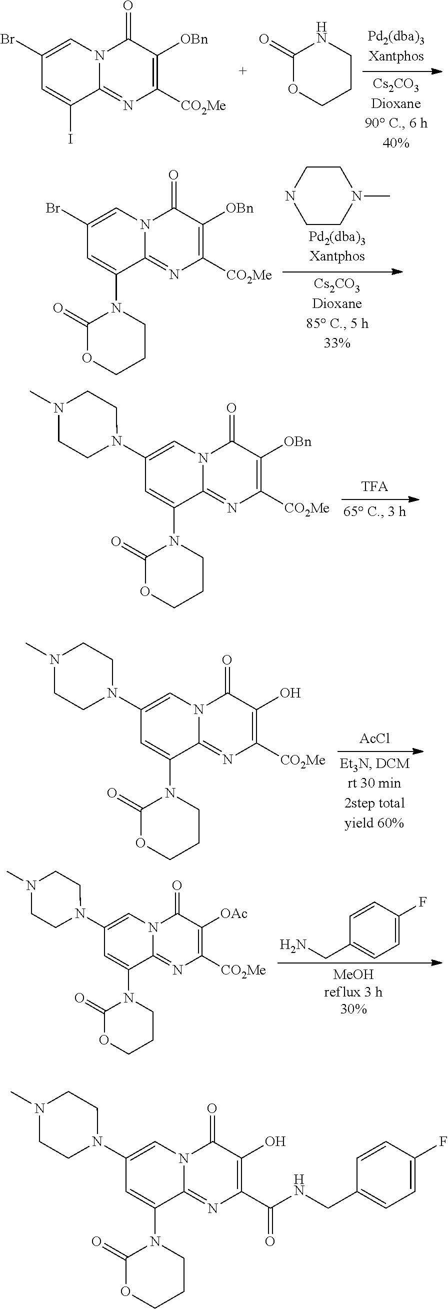

[1,3]Oxazinan-2-one was made according to the literature: Journal of Heterocyclic Chemistry, 1966, 3(1), 84-89, using starting material described in synthetic scheme 1.

### Example 1.1

### Preparation of Methyl-3-(benzyloxy)-7-bromo-4-oxo-9-(2-oxo-1,3-oxazinan-3-yl)-4H-pyrido[1,2-a]pyrimidine-2-carboxylate

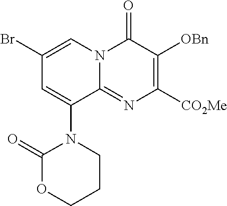 [SMILES: COC(=O)c1nc2c(N3CCCOC3=O)cc(Br)cn2c(=O)c1OCc1ccccc1]

The mixture of [1,3]Oxazinan-2-one (235 mg, 2.23 mmol), methyl 3-(benzyloxy)-7-bromo-9-iodo-4-oxo-4H-pyrido[1,2-a]pyrimidine-2-carboxylate (1 g, 1.94 mmol), Pd₂dba₃ (179 mg, 0.19 mmol), xantphos (224 mg, 0.39 mmol) and Cs₂CO₃ (950 mg, 2.9 mmol) were mixed in dioxane (10 ml). The mixture was heated under N₂ atmosphere at 90° C. for 6 h. After cooling down to room temperature, dioxane was removed in vacuo. The residue was purified by column chromatography (EA/PE=1/1) to give the desired product (350 mg, 40%).

¹H NMR (300 M Hz, CDCl₃) δ 9.08 (d, J=2.1 Hz, 1H), 7.79 (d, J=2.1 Hz, 1H), 7.55-7.45 (m, 2H), 7.44-7.31 (m, 3H), 5.34 (s, 2H), 4.53 (t, J=5.1 Hz, 2H), 3.91 (s, 3H), 3.84-3.64 (m, 2H), 2.34-2.19 (m, 2H).

MS (ESI⁺) m/z 488 (M[Br⁷⁹]+1), 490 (M[Br⁸¹]+1)

### Example 1.2

### Preparation of Methyl-3-(benzyloxy)-7-(4-methylpiperazin-1-yl)-4-oxo-9-(2-oxo-1,3-oxazinan-3-yl)-4H-pyrido[1,2-a]pyrimidine-2-carboxylate

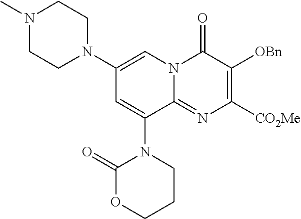 [SMILES: COC(=O)c1nc2c(N3CCCOC3=O)cc(N3CCN(C)CC3)cn2c(=O)c1OCc1ccccc1]

The product from example 1.1 (300 mg, 0.61 mmol), 1-methylpiperazine (123 mg, 1.23 mmol), Pd₂ dba₃ (56 mg, 0.06 mmol), xantphos (69 mg, 0.12 mmol) and Cs₂CO₃ (298 mg, 0.92 mmol) were mixed in dioxane (3 ml). The mixture was heated at 85° C. under N₂ atmosphere for 6 h. After cooling down to room temperature, dioxane was removed in vacuo. The residue was purified by column chromatography (DCM/MeOH=20/1) to give the desired product (120 mg, 33%).

¹H NMR (300 M Hz, CDCl₃) δ 9.08 (d, J=2.1 Hz, 1H), 7.79 (d, J=2.1 Hz, 1H), 7.59-7.49 (m, 2H), 7.44-7.31 (m, 3H), 5.30 (s, 2H), 4.53 (t, J=4.8 Hz, 2H), 3.89 (s, 3H), 3.84-3.70 (m, 2H), 3.36-3.20 (m, 4H), 2.67-2.51 (m, 4H), 2.38 (s, 3H), 2.34-2.18 (m, 2H).

MS (ESI⁺) m/z 508 (M+1)

### Example 1.3

### Preparation of Methyl-3-acetoxy-7-(4-methylpiperazin-1-yl)-4-oxo-9-(2-oxo-1,3-oxazinan-3-yl)-4H-pyrido[1,2-a]pyrimidine-2-carboxylate

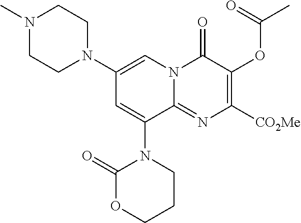 [SMILES: COC(=O)c1nc2c(N3CCCOC3=O)cc(N3CCN(C)CC3)cn2c(=O)c1OC(C)=O]

The solution of the product from example 1.2 (120 mg, 0.24 mmol) in TFA (2 ml) was heated at 65° C. for 2 h. After cooling down to room temperature, TFA was removed in vacuo to give the crude product methyl 3-hydroxy-7-(4-methylpiperazin-1-yl)-4-oxo-9-(2-oxo-1,3-oxazinan-3-yl)-4H-pyrido[1,2-a]pyrimidine-2-carboxylate, which was used directly in the acetylation step.

To the solution of the crude product from last step and TEA (119 mg, 1.18 mmol) in DCM (3 ml), was added AcCl (185 mg, 2.36 mmol) dropwise. The mixture was stirred at room temperature for 1 h, and then water was added, followed by EA. The organic phase was washed with water 3 times, dried over Na₂SO₄, and concentrated in vacuo. The residue was purified by column chromatography (DCM/MeOH=20/1) to give the desired product (62 mg, two step total 60%).

¹H NMR (300 M Hz, CDCl₃) δ 8.30 (s, 1H), 8.79 (s, 1H), 4.56 (t, J=4.8 Hz, 2H), 3.95 (s, 3H), 3.89-3.73 (m, 2H), 3.48-3.28 (m, 4H), 2.80-2.58 (m, 4H), 2.46 (s, 3H), 2.41 (s, 3H), 2.31 (t, J=4.9, 2H).

MS (ESI⁺) m/z 460 (M+1)

### Example 1.4

### Preparation of N-(4-fluorobenzyl)-3-hydroxy-7-(4-methylpiperazin-1-yl)-4-oxo-9-(2-oxo-1,3-oxazinan-3-yl)-4H-pyrido[1,2-a]pyrimidine-2-carboxamide

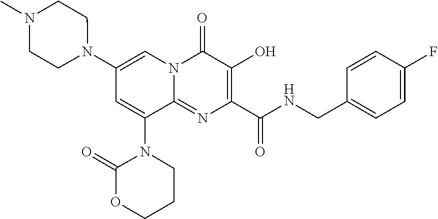 [SMILES: CN1CCN(c2cc(N3CCCOC3=O)c3nc(C(=O)NCc4ccc(F)cc4)c(O)c(=O)n3c2)CC1]

The solution of the product from example 1.3 (60 mg, 0.13 mmol) and (4-fluorophenyl)methan amine (84 mg, 0.67 mmol) in MeOH (1 ml) was heated at 65° C. for 4 h. After cooling down to room temperature, MeOH was removed in vacuo to give the crude product, which was further purified on preparative HPLC using 0.1% formic acid as eluent to give the desired product (20 mg, 30%).

¹H NMR (300 M Hz, CDCl₃) δ 8.17 (m, 1H), 7.86 (m, 1H), 7.52 (m, 1H), 7.46-7.34 (m, 2H), 7.17-7.01 (m, 2H), 4.73-4.46 (m, 2H), 4.37-4.07 (m, 2H), 3.74-3.52 (m, 2H), 3.40-3.17 (m, 4H), 2.78-2.59 (m, 4H), 2.43 (s, 3H), 2.15-1.89 (m, 2H).

MS (ESI⁺) m/z 511 (M+1)

### Example 2

### Preparation of 3-Hydroxy-7-(4-methyl-piperazin-1-yl)-4-oxo-9-(2-oxo-pyrrolidin-1-yl)-4H-pyrido[1,2-a]pyrimidine-2-carboxylic acid 4-fluoro-benzylamide

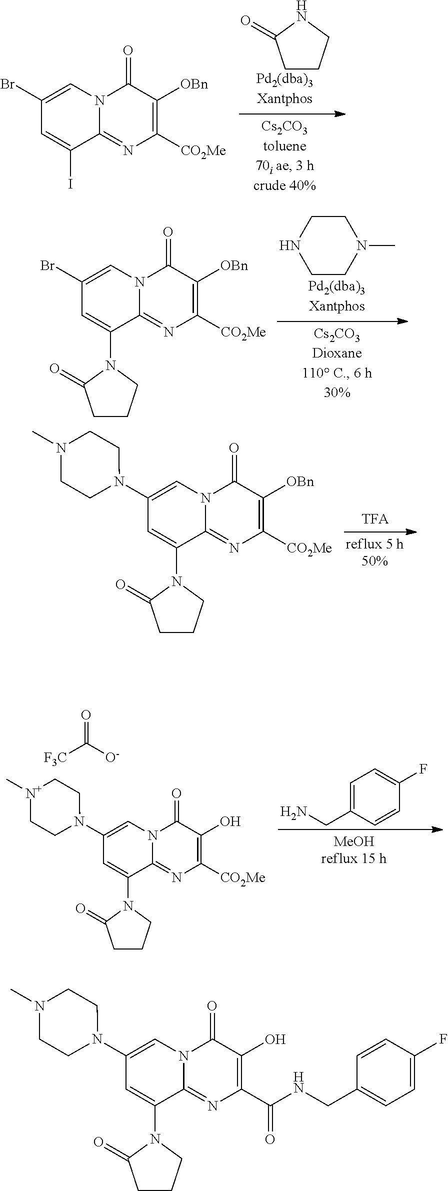

### Example 2.1

### Preparation of 3-Benzyloxy-7-bromo-4-oxo-9-(2-oxo-pyrrolidin-1-yl)-4H-pyrido[1,2-a]pyrimidine-2-carboxylic acid methyl ester

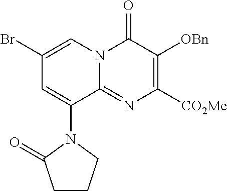 [SMILES: COC(=O)c1nc2c(N3CCCC3=O)cc(Br)cn2c(=O)c1OCc1ccccc1]

Compound prepared as set out in step 1 of the scheme for example 2, the crude product was used in the next step.

MS (ESI⁺) m/z 472 (M[Br⁷⁹]+1), 474 (M[Br⁸¹]+1)

### Example 2.2

### Preparation of 3-Benzyloxy-7-(4-methyl-piperazin-1-yl)-4-oxo-9-(2-oxo-pyrrolidin-1-yl)-4H-pyrido[1,2-a]pyrimidine-2-carboxylic acid methyl ester

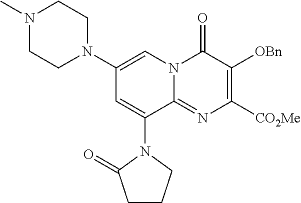 [SMILES: COC(=O)c1nc2c(N3CCCC3=O)cc(N3CCN(C)CC3)cn2c(=O)c1OCc1ccccc1]

This compound was prepared by adapting the procedure described in Example 1.2.

¹H NMR (300 MHz, CDCl₃) δ 2.17-2.27 (m, 2H), 2.39 (s, 3H), 2.55-2.75 (m, 6H), 3.25-3.35 (m, 4H), 3.89 (s, 3H), 4.17 (t, J=7.1 Hz, 2H), 5.30 (s, 2H), 7.30-7.42 (m, 3H), 7.46-7.56 (m, 2H), 7.78 (d, J=2.5 Hz, 1H), 8.29 (d, J=2.4 Hz, 1H).

MS (ESI⁺) m/z 492 (M+1)

### Example 2.3

### Preparation of Trifluoro-acetate-4-[3-hydroxy-2-methoxycarbonyl-4-oxo-9-(2-oxo-pyrrolidin-1-yl)-4H-pyrido[1,2-a]pyrimidin-7-yl]-1-methyl-piperazin-1-ium

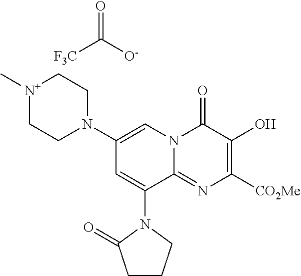 [SMILES: O=C([O-])C(F)(F)F ; COC(=O)c1nc2c(N3CCCC3=O)cc(N3CC[NH+](C)CC3)cn2c(=O)c1O]

The mixture of the product from example 2.2 (300 mg, 0.611 mmol) in TFA (5 ml) was heated to reflux for 5 h. After cooling down to room temperature, the mixture was concentrated in vacuo. The resulting residue was recrystallized from a mixed solvent of PE/EA/MeOH (10/3/1) to give the title compound (200 mg, 81.6%).

¹H NMR (300 MHz, DMSO-d⁶) δ 2.08-2.22 (m, 2H), 2.45-2.60 (m, 2H, overlap), 2.87 (s, 3H), 2.95-3.95 (m, 8H), 3.88 (s, 3H), 3.97 (t, J^{=7.0} Hz, 2H), 7.86 (d, J^{=2.5} Hz, 1H), 8.04 (d, J=2.3 Hz, 1H), 9.80-10.02 (brs, 1H), 10.30 (s, 1H)

MS (ESI⁺) m/z 402 (M-TFA+1)

### Example 2.4

### Preparation of 3-Hydroxy-7-(4-methyl-piperazin-1-yl)-4-oxo-9-(2-oxo-pyrrolidin-1-yl)-4H-pyrido[1,2-a]pyrimidine-2-carboxylic acid 4-fluoro-benzylamide

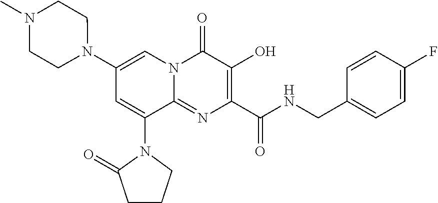 [SMILES: CN1CCN(c2cc(N3CCCC3=O)c3nc(C(=O)NCc4ccc(F)cc4)c(O)c(=O)n3c2)CC1]

The product from example 2.3 (100 mg, 0.25 mmol) was suspended in MeOH (5 ml) under N₂ atmosphere, and then 4-fluorobenzylamine (100 mg, 1.00 mmol) was added. The mixture was heated at 75° C. for 15 hours. After cooling down to room temperature, the mixture was concentrated in vacuo. The residue was sonicated in MeOH (2 ml). The resulting solids were collected by filtration, washed with cold methanol and dried in vacuo to afford the desired product as yellow solids (70 mg, 57%).

¹H NMR (300 MHz, CDCl₃) δ 2.00-2.15 (m, 2H), 2.37 (s, 3H), 2.47 (t, J=8.2 Hz, 2H), 2.54-2.65 (m, 4H), 3.18-3.30 (m, 4H), 3.85 (t, J=6.9 Hz, 2H), 4.60 (d, J=5.9 Hz, 2H), 7.08 (t, J=8.9 Hz, 2H), 7.30-7.40 (m, 2H), 7.51 (d, J=2.6 Hz, 1H), 7.82 (t, J=5.4 Hz, 1H), 8.15 (d, J^{=2.6} Hz, 1H), 11.65-11.90 (brs, 1H).

MS (ESI⁺) m/z 495 (M+1)

HPLC 98.4%

### Example 3

### The preparation of 3-Hydroxy-9-(5-methyl-1,1-dioxo-1,6-[1,2,5]thiadiazolidin-2-yl)-7-(4-methyl-piperazin-1-yl)-4-oxo-4H-pyrido[1,2-a]pyrimidine-2-carboxylic acid 4-fluoro-benzylamide

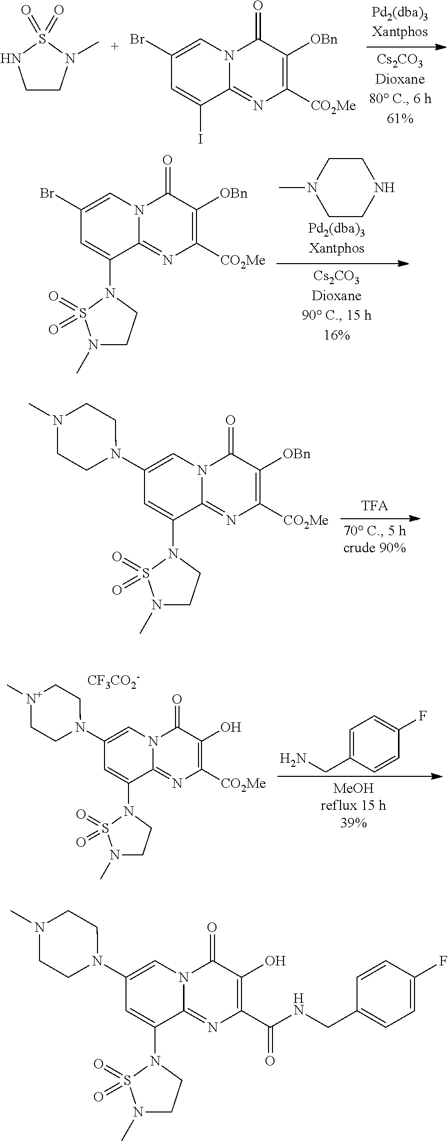

The cyclic sulfonamide starting material was prepared according to the literature: Eur. J. Med. Chem. 2007, 42 (9), 1176-1183. The target compound was prepared by adapting the procedure described in Example 2.

### Example 3.1

### Preparation of 3-Benzyloxy-7-bromo-9-(5-methyl-1,1-dioxo-1,6-[1,2,5]thiadiazolidin-2-yl)-4-oxo-4H-pyrido[1,2-a]pyrimidine-2-carboxylic acid methyl ester

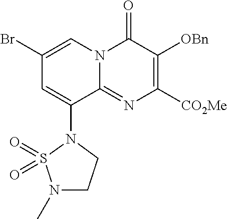 [SMILES: COC(=O)c1nc2c(N3CCN(C)S3(=O)=O)cc(Br)cn2c(=O)c1OCc1ccccc1]

Compound prepared as set out in step 1 of the scheme for example 3.

¹H NMR (300 MHz, CDCl₃) δ 2.89 (s, 3H), 3.57 (t, J=6.6 Hz, 2H), 3.89 (s, 3H), 4.37 (t, J=6.3 Hz, 2H,), 5.34 (s, 2H), 7.32-7.42 (m, 3H), 7.48 (dd, J=8.4, 1.8 Hz, 2H), 7.97 (d, J=2.3 Hz, 1H), 8.98 (d, J=1.8 Hz, 1H)

MS (ESI⁺) m/z 523 (M[Br⁷⁹]+1), 525 (M[Br⁸¹]+1)

### Example 3.2

### Preparation of 3-Benzyloxy-9-(5-methyl-1,1-dioxo-1,6-[1,2,5]thiadiazolidin-2-yl)-7-(4-methyl-piperazin-1-yl)-4-oxo-4H-pyrido[1,2-a]pyrimidine-2-carboxylic acid methyl ester

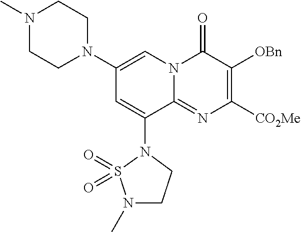 [SMILES: COC(=O)c1nc2c(N3CCN(C)S3(=O)=O)cc(N3CCN(C)CC3)cn2c(=O)c1OCc1ccccc1]

Compound prepared as set out in step 2 of the scheme for example 3.

¹H NMR (300 MHz, CDCl₃) δ 2.38 (s, 3H), 2.55-2.65 (m, 4H), 2.89 (s, 3H), 3.24-3.35 (m, 4H), 3.59 (t, J=6.4 Hz, 2H), 3.88 (s, 3H), 4.39 (t, J=6.7 Hz, 2H), 5.30 (s, 2H), 7.30-7.45 (m, 3H), 7.47-7.57 (m, 2H), 7.92 (d, J=2.3 Hz, 1H), 8.25 (d, J=2.1 Hz, 1H).

MS (ESI⁺) m/z 543 (M+1)

### Example 3.3

### Preparation of Trifluoro-acetate-4-[3-hydroxy-2-methoxycarbonyl-9-(5-methyl-1,1-dioxo-1,6-[1,2,5]thiadiazolidin-2-yl)-4-oxo-4H-pyrido[1,2-a]pyrimidin-7-yl]-1-methyl-piperazin-1-ium

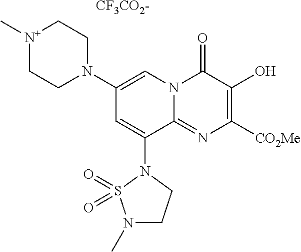 [SMILES: COC(=O)c1nc2c(N3CCN(C)S3(=O)=O)cc(N3CC[NH+](C)CC3)cn2c(=O)c1O ; "CF3CO2-"]

Compound prepared as set out in step 3 of the scheme for example 3, and the crude product was used in the next step.

MS (ESI⁺) m/z 453 (M-TFA+1).

### Example 3.4

### Preparation of 3-Hydroxy-9-(5-methyl-1,1-dioxo-1,6-[1,2,5]thiadiazolidin-2-yl)-7-(4-methyl-piperazin-1-yl)-4-oxo-4H-pyrido[1,2-a]pyrimidine-2-carboxylic acid 4-fluoro-benzylamide

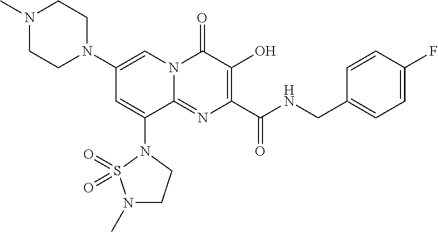 [SMILES: CN1CCN(c2cc(N3CCN(C)S3(=O)=O)c3nc(C(=O)NCc4ccc(F)cc4)c(O)c(=O)n3c2)CC1]

Compound prepared as set out in step 4 of the scheme for example 3, using the product of example 3.3.

¹H NMR (300 MHz, CDCl₃) δ 2.38 (s, 3H), 2.53-2.67 (m, 4H), 2.76 (s, 3H), 3.18-3.30 (m, 4H), 3.50 (t, J=5.8 Hz 2H,), 3.94 (t, J=5.8 Hz, 2H), 4.59 (t, J=5.4 Hz, 2H), 6.95-7.11 (m, 3H), 7.32-7.44 (m, 2H), 8.06 (d, J=1.6 Hz, 1H), 9.38-9.50 (brs, 1H), 12.15-12.35 (brs, 1H).

MS (ESI⁺) m/z 546 (M+1)

HPLC=96.7%

### Example 4

### Preparation of 3-Hydroxy-4-oxo-9-(2-oxo-piperazin-1-yl)-7-pyrrolidin-1-yl-4H-pyrido[1,2-a]pyrimidine-2-carboxylic acid 4-fluoro-benzylamide

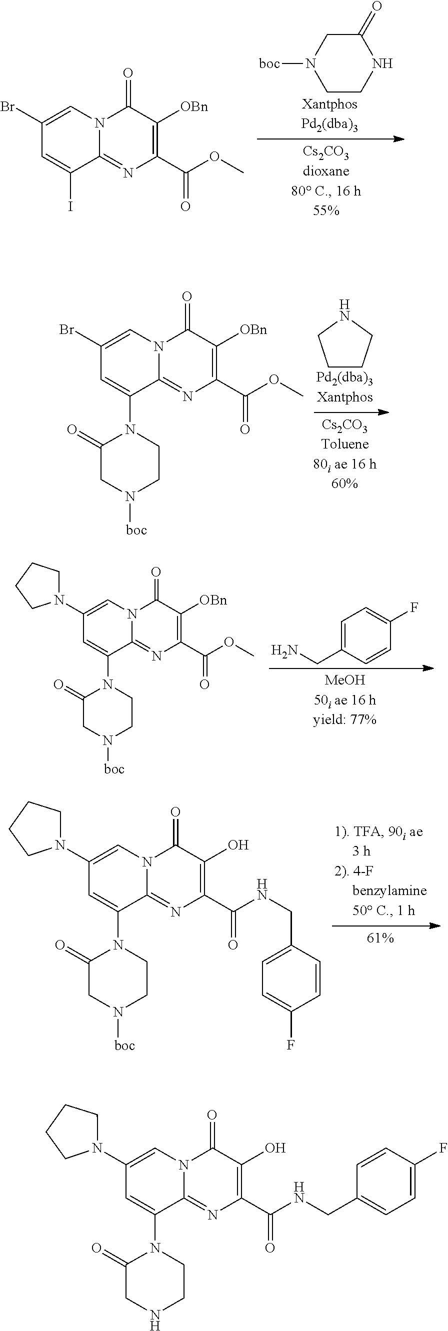

### Example 4.1

### Preparation of 3-Benzyloxy-7-bromo-9-(4-tert-butoxycarbonyl-2-oxo-piperazin-1-yl)-4-oxo-4H-pyrido[1,2-a]pyrimidine-2-carboxylic acid methyl ester

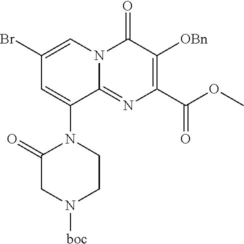

This compound was prepared as set out in step 1 of the scheme for example 4.

¹H NMR (300 MHz, CDCl₃) δ 9.09 (d, J=2.1 Hz, 1H), 7.73 (d, J=2.1 Hz, 1H), 7.53-7.43 (m, 2H), 7.42-7.31 (m, 3H), 5.33 (s, 2H), 4.30 (s, 2H), 3.93-3.72 (m, 7H), 1.51 (s, 9H)

MS (ESI⁺) m/z 587 (M[Br⁷⁹]+1), 589 (M[Br⁸¹]+1)

### Example 4.2

### Preparation of 3-Benzyloxy-9-(4-tert-butoxycarbonyl-2-oxo-piperazin-1-yl)-4-oxo-7-pyrrolidin-1-yl-4H-pyrido[1,2-a]pyrimidine-2-carboxylic acid methyl ester

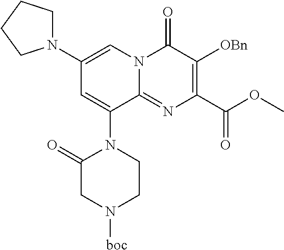

This compound was prepared by adapting the procedure described in Example 1.2.

¹H-NMR (300 MHz, CDCl₃) δ 8.06 (d, J=2.0 Hz, 1H), 7.48-7.56 (m, 2H), 7.43 (d, J=2.0 Hz, 1H), 7.30-7.41 (m, 3H), 5.29 (s, 2H), 4.32 (s, 2H), 3.70-3.96 (m, 7H), 3.31-3.49 (m, 4H), 2.01-2.15 (m, 4H), 1.51 (s, 9H)).

MS (ESI⁺) m/z 578 (M+1)

### Example 4.3

### Preparation of 4-[3-Benzyloxy-2-(4-fluoro-benzylcarbamoyl)-4-oxo-7-pyrrolidin-1-yl-4H-pyrido[1,2-a]pyrimidin-9-yl]-3-oxo-piperazine-1-carboxylic acid tert-butyl ester

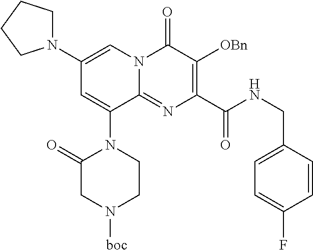

The product from Example 4.2 (224 mg, 0.39 mmol) 4-fluorobenzylamine (3.50 g, 28.00 mmol) were mixed in methanol (4 ml). The mixture was heated at 50° C. for 20 hours. After cooling down to room temperature, the mixture was diluted with DCM (20 ml), washed with 0.5 N HCl (10 ml×2) and water (20 ml×3) successively, dried over Na₂SO₄ and concentrated in vacuo. The residue was purified on column chromatography using a mixed solvent EA/PE (1:1) as eluent to give the desired product (202 mg, 77%).

¹H NMR (300 MHz, CDCl₃) δ 8.02 (d, J=2.2 Hz, 1H), 7.67-7.59 (m, 1H), 7.50-7.44 (m, 2H), 7.42 (d, J=2.7 Hz, 1H), 7.38-7.30 (m, 3H), 7.24-7.20 (m, 2H), 7.05 (t, J=8.7 Hz, 2H), 5.30 (s, 2H), 4.52 (d, J=5.9 Hz, 2H), 4.23 (s, 2H), 3.85-3.75 (m, 4H), 3.50-3.30 (m, 4H), 2.15-2.05 (m, 4H), 1.51 (s, 9H).

MS (ESI⁺) m/z 671 (M+1)

### Example 4.4

### Preparation of 3-Hydroxy-4-oxo-9-(2-oxo-piperazin-1-yl)-7-pyrrolidin-1-yl-4H-pyrido[1,2-a]pyrimidine-2-carboxylic acid 4-fluoro-benzylamide

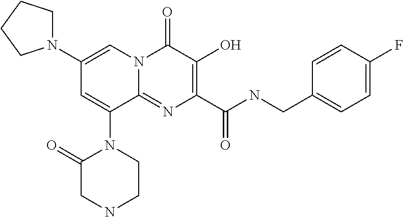 [SMILES: O=C(NCc1ccc(F)cc1)c1nc2c(N3CCNCC3=O)cc(N3CCCC3)cn2c(=O)c1O]

The product from example 4.3 (100 mg, 0.15 mmol) was mixed in TFA (2 ml). The mixture was heated at 90° C. for 3 hours. After cooling down to room temperature, the mixture was concentrated into dryness. The residue was sonicated in EA. The resulting solids were collected by filtration, washed with EA (2 ml×2), dried in vacuo. The crude TFA salt was mixed in methanol (2 ml) and then 4-fluorobenzylamine (100 mg, 0.80 mmol) was added. The mixture was heated at 50° C. for 1 hour. After cooling down to room temperature, the solids were collected by filtration, washed with anhydrous methanol (2 ml×3) and dried in vacuo to give the final target (44 mg. 61%).

¹H NMR (300 MHz, CDCl₃) δ 11.75-11.40 (brs, 1H), 8.13 (t, J=5.1 Hz, 1H), 7.90 (d, J=2.3 Hz, 1H), 7.41-7.32 (m, 2H), 7.30 (d, J=2.6 Hz, 1H), 7.08 (t, J=8.6 Hz, 2H), 4.59 (d, J=5.4 Hz, 2H), 3.83-3.45 (m, 4H), 3.43-3.29 (m, 4H), 3.18-2.97 (brs, 2H), 2.15-1.94 (m, 4H).

MS (ESI⁺) m/z 481 (M+1), 503 (M+23)

HPLC 97.48%

### Example 5

### Preparation of 3-Hydroxy-9-(methanesulfonyl-methyl-amino)-7-(4-methyl-piperazin-1-yl)-4-oxo-4H-pyrido[1,2-a]pyrimidine-2-carboxylic acid 4-fluoro-benzylamide

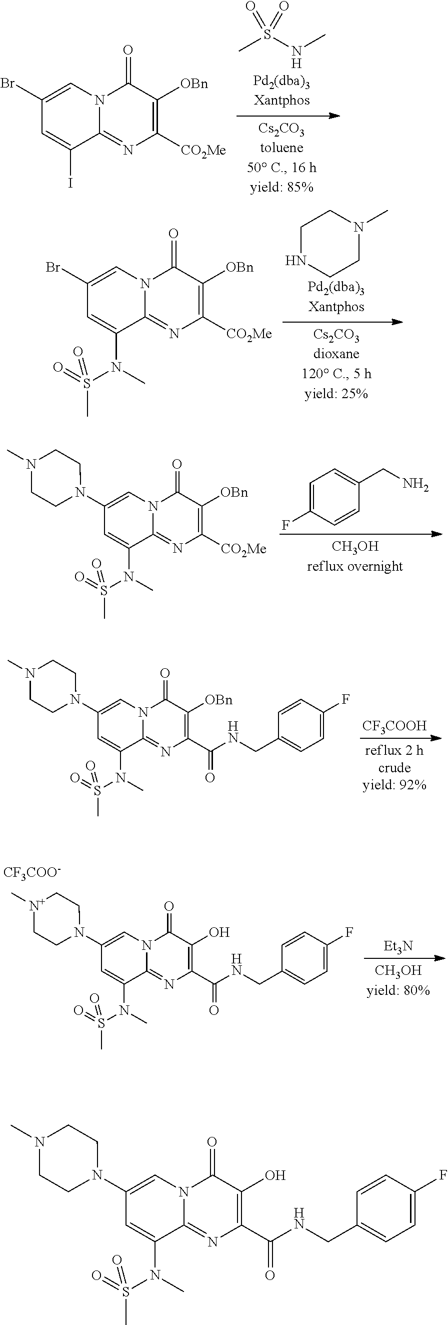

This target was prepared by adapting the procedure described in Example 4, except that TEA was used instead of 4-fluorobenzylamine in the de-salting step.

### Example 5.1

### Preparation of 3-Benzyloxy-7-bromo-9-(methanesulfonyl-methyl-amino)-4-oxo-4H-pyrido[1,2-a]pyrimidine-2-carboxylic acid methyl ester

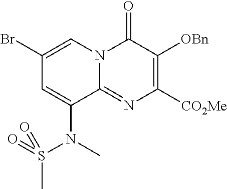 [SMILES: COC(=O)c1nc2c(N(C)S(C)(=O)=O)cc(Br)cn2c(=O)c1OCc1ccccc1]

This compound was prepared by adapting the procedure described in example 4.1.

¹H NMR (300 MHz, CDCl₃) δ 9.07 (d, J=2.1 Hz, 1H), 7.84 (d, J=2.1 Hz, 1H), 7.52-7.45 (m, 2H), 7.42-7.33 (m, 3H), 5.35 (s, 2H), 3.89 (s, 3H), 3.39 (s, 3H), 3.22 (s, 3H).

MS (ESI⁺) m/z 496 (M[Br⁷⁹]+1), 498 (M[Br⁸¹]+1)

### Example 5.2

### Preparation of 3-Benzyloxy-9-(methanesulfonyl-methyl-amino)-7-(4-methyl-piperazin-1-yl)-4-oxo-4H-pyrido[1,2-a]pyrimidine-2-carboxylic acid methyl ester

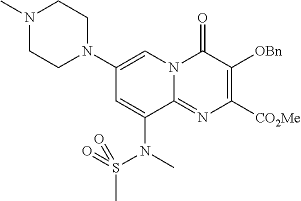 [SMILES: COC(=O)c1nc2c(N(C)S(C)(=O)=O)cc(N3CCN(C)CC3)cn2c(=O)c1OCc1ccccc1]

This compound was prepared by adapting the procedure described in example 4.2.

¹H NMR (300 MHz, CDCl₃) δ 8.30 (d, J=2.4 Hz, 1H), 7.77 (d, J=2.4 Hz, 1H), 7.49-7.56 (m, 2H), 7.41-7.31 (m, 3H), 5.31 (s, 2H), 3.88 (s, 3H), 3.41 (s, 3H), 3.30-3.26 (m, 4H), 3.20 (s, 3H), 2.67-2.63 (m, 4H), 2.41 (s, 3H).

MS (ESI⁺) m/z 516 (M+1)

### Example 5.3

### Preparation of 3-Benzyloxy-9-(methanesulfonyl-methyl-amino)-7-(4-methyl-piperazin-1-yl)-4-oxo-4H-pyrido[1,2-a]pyrimidine-2-carboxylic acid 4-fluoro-benzylamide

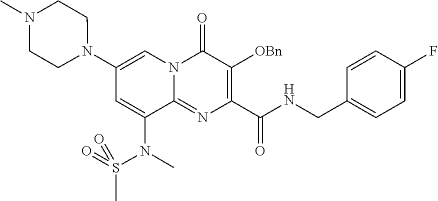 [SMILES: CN1CCN(c2cc(N(C)S(C)(=O)=O)c3nc(C(=O)NCc4ccc(F)cc4)c(OCc4ccccc4)c(=O)n3c2)CC1]

This compound was prepared by adapting the procedure described in example 4.3.

¹H NMR (300 MHz, CDCl₃) δ 8.24 (d, J=2.4 Hz, 1H), 8.10 (t, J=5.4 Hz, 1H), 7.75 (d, J=2.4 Hz, 1H), 7.39-7.27 (m, 5H), 7.16-7.20 (m, 2H), 7.09-6.96 (m, 2H), 5.30 (s, 2H), 4.48 (d, J=5.7 Hz, 2H), 3.43 (s, 3H), 3.37 (s, 3H), 3.33-3.24 (m, 4H), 2.63-2.59 (s, 4H), 2.38 (s, 3H).

MS (ESI⁺) m/z 609 (M+1)

### Example 5.4

### Preparation of 3-Hydroxy-9-(methanesulfonyl-methyl-amino)-7-(4-methyl-piperazin-1-yl)-4-oxo-4H-pyrido[1,2-a]pyrimidine-2-carboxylic acid 4-fluoro-benzylamide

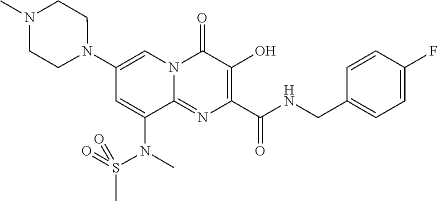 [SMILES: CN1CCN(c2cc(N(C)S(C)(=O)=O)c3nc(C(=O)NCc4ccc(F)cc4)c(O)c(=O)n3c2)CC1]

This compound was prepared by adapting the procedure described in example 4.4.

¹H NMR (300 MHz, CDCl₃) δ 8.44 (t, J=6.3 Hz, 1H), 8.15 (d, J=2.4 Hz, 1H), 7.53 (d, J=2.4 Hz, 1H), 7.37-7.29 (m, 2H), 7.05 (t, J=8.7 Hz, 2H), 4.61 (d, J=6.3 Hz, 2H), 3.26 (s, 7H), 2.99 (s, 3H), 2.62 (s, 4H), 2.39 (s, 3H).

MS (ESI⁺) m/z 519 (M+1)

### Example 6

### Preparation of Trifluoro-acetate 4-[2-(4-fluoro-benzylcarbamoyl)-3-hydroxy-9-(methanesulfonyl-methyl-amino)-4-oxo-4H-pyrido[1,2-a]pyrimidin-7-yl]-1-isopropyl-piperazin-1-ium

This compound was made by adapting the procedure described in Example 4, except that no de-salting was done.

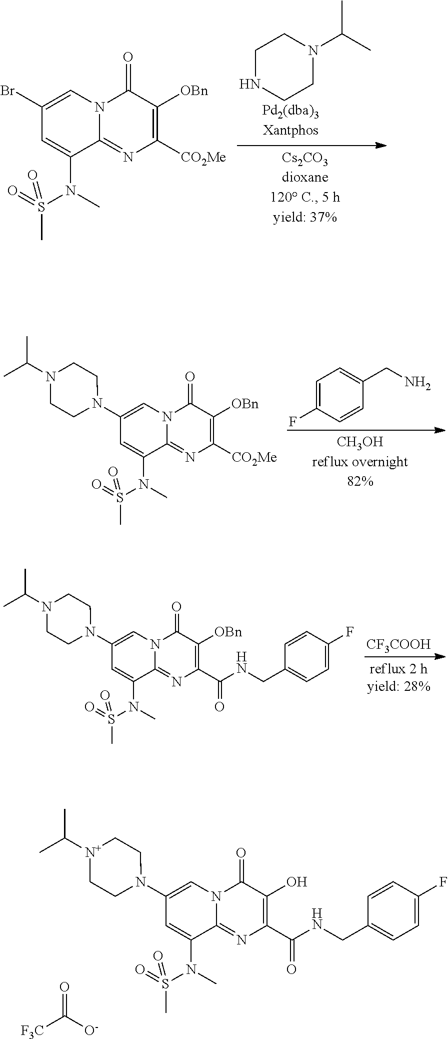

### Example 6.1

### Preparation of 3-Benzyloxy-7-(4-isopropyl-piperazin-1-yl)-9-(methanesulfonyl-methyl-amino)-4-oxo-4H-pyrido[1,2-a]pyrimidine-2-carboxylic acid methyl ester

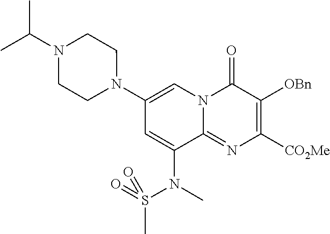 [SMILES: COC(=O)c1nc2c(N(C)S(C)(=O)=O)cc(N3CCN(C(C)C)CC3)cn2c(=O)c1OCc1ccccc1]

This compound was prepared by adapting the procedure described in example 1.2.

¹H NMR (300 MHz, CDCl₃) δ 8.29 (d, J=2.2 Hz, 1H), 7.77 (d, J=2.4 Hz, 1H), 7.52 (d, J=6.6 Hz, 2H), 7.37 (dd, J=15.0, 7.4 Hz, 3H), 5.31 (s, 2H), 3.88 (s, 3H), 3.41 (s, 3H), 3.30 (s, 4H), 3.21 (s, 3H), 2.72 (s, 5H), 1.12 (s, 6H).

MS (ESI⁺) m/z 544 (M+1)

### Example 6.2

### Preparation of 3-Benzyloxy-7-(4-isopropyl-piperazin-1-yl)-9-(methanesulfonyl-methyl-amino)-4-oxo-4H-pyrido[1,2-a]pyrimidine-2-carboxylic acid 4-fluoro-benzylamide

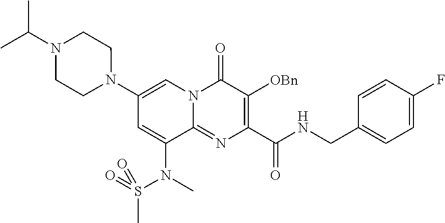 [SMILES: CC(C)N1CCN(c2cc(N(C)S(C)(=O)=O)c3nc(C(=O)NCc4ccc(F)cc4)c(OCc4ccccc4)c(=O)n3c2)CC1]

This compound was prepared by adapting the procedure described in example 4.3.

¹H NMR (300 MHz, CDCl₃) δ 8.23 (d, J=2.7 Hz, 1H), 8.11 (m, 1H), 7.76 (d, J=2.4 Hz, 1H), 7.33 (s, 5H), 7.21-7.13 (m, 2H), 6.98 (t, J=8.7 Hz, 2H), 5.29 (s, 2H), 4.48 (d, J=5.4 Hz, 2H), 3.43 (s, 3H), 3.38 (s, 3H), 3.37-3.30 (m, 4H), 2.75-2.70 (m, 5H), 1.14-1.08 (s, 6H).

MS (ESI⁺) m/z 637 (M+1)

### Example 6.3

### Preparation of Trifluoro-acetate4-[2-(4-fluoro-benzylcarbamoyl)-3-hydroxy-9-(methanesulfonyl-methyl-amino)-4-oxo-4H-pyrido[1,2-a]pyrimidin-7-yl]-1-isopropyl-piperazin-1-ium

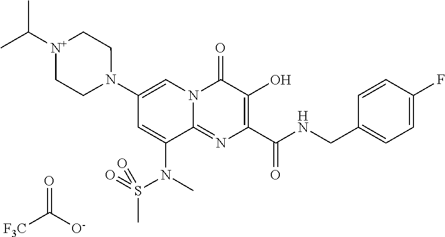 [SMILES: CC(C)[NH+]1CCN(c2cc(N(C)S(C)(=O)=O)c3nc(C(=O)NCc4ccc(F)cc4)c(O)c(=O)n3c2)CC1 ; O=C([O-])C(F)(F)F]

This compound was prepared by adapting the procedure described in example 4.4, except that no de-salting was done.

¹H NMR (300 MHz, CDCl₃) δ 8.49 (m, 1H), 8.12 (d, J=2.1 Hz, 1H), 7.54 (d, J=2.1 Hz, 1H), 7.38-7.29 (m, 2H), 7.04 (t, J=8.7 Hz, 2H), 4.60 (d, J=6 Hz, 2H), 3.28-3.22 (m, 7H), 3.01 (s, 3H), 2.76-2.70 (m, 5H), 1.09 (d, J=6.6 Hz, 6H).

MS (ESI⁺) m/z 547 (M-TFA+1)

### Example 7

### Preparation of 7-(dimethylamino)-N-(4-fluorobenzyl)-3-hydroxy-4-oxo-9-(2-oxooxazolidin-3-yl)-4H-pyrid[1,2-a]pyrimidine-2-carboxamide

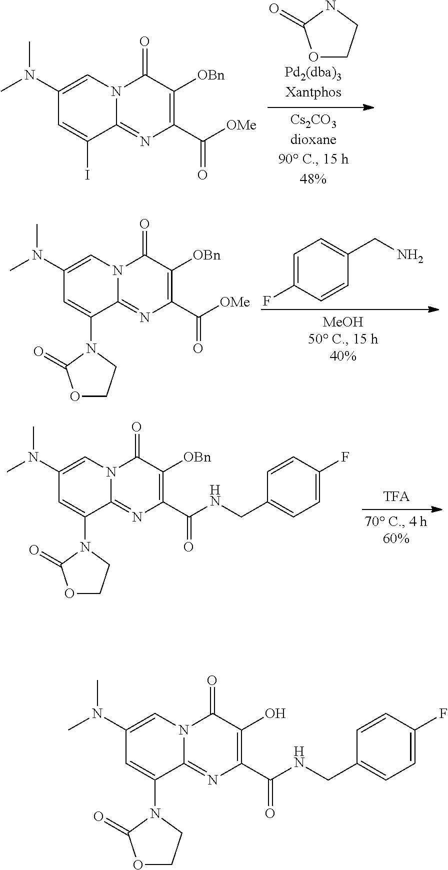

The starting material 3-benzyloxy-7-dimethylamino-9-iodo-4-oxo-4H-pyrido[1,2-a]pyrimidine-2-carboxylic acid methyl ester was prepared by a similar method to the procedure described under the heading “Synthesis of the starting material 3-benzyloxy-7-dimethylamino-9-iodo-4-oxo-4H-pyrido[1,2-a]pyrimidine-2-carboxylic acid methyl ester” above.

As can be seen from the synthetic scheme, the substituent at the 7-position was fixed at the core stage and the substituent at the 9-position was introduced by coupling reaction. Amide reaction followed by deprotection of Bn gave the desired product.

### Example 7.1

### Preparation of methyl 3-(benzyloxy)-7-(dimethylamino)-4-oxo-9-(2-oxooxazolidin-3-yl)-4H-pyrido[1,2-a]pyrimidine-2-carboxylate

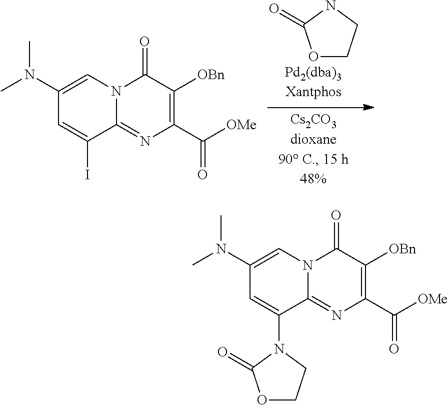

This compound was prepared by adapting the procedure in Example 1.1, using the product of step 5 of the synthetic examples set out above.

¹H NMR (300 MHz, CDCl₃) δ 8.15 (d, J=2.7 Hz, 1H), 7.86 (d, J=2.7 Hz, 1H), 7.56-7.50 (m, 2H), 7.43-7.32 (m, 3H), 5.31 (s, 2H), 4.60-4.50 (m, 4H), 3.90 (s, 3H), 3.08 (s, 6H).

MS (ESI⁺) m/z 439 (M+1), 461 (M+23)

### Example 7.2

### Preparation of 3-(benzyloxy)-7-(dimethylamino)-N-(4-fluorobenzyl)-4-oxo-9-(2-oxooxazolidin-3-yl)-4H-pyrido[1,2-a]pyrimidine-2-carboxamide

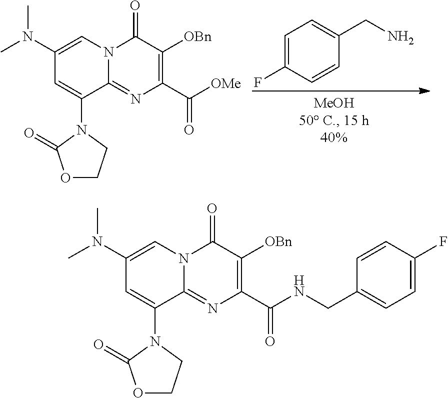

This compound was prepared by adapting the procedure in Example 4.3.

¹H NMR (300 MHz, CDCl₃) δ 8.09 (d, J=3.0 Hz, 1H), 7.98-7.87 (m, 2H), 7.42-7.36 (m, 2H), 7.36-7.30 (m, 3H), 7.27-7.21 (m, 2H), 6.99 (t, J=8.8 Hz, 2H), 5.32 (s, 2H), 4.64-4.44 (m, 6H), 3.08 (s, 6H).

MS (ESI⁺) m/z 532 (M+1), 554 (M+23)

### Example 7.3

### Preparation of 7-(dimethylamino)-N-(4-fluorobenzyl)-3-hydroxy-4-oxo-9-(2-oxooxazolidin-3-yl)-4H-pyrido[1,2-a]pyrimidine-2-carboxamide

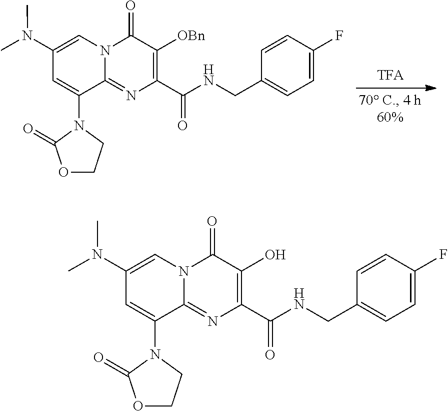 [SMILES: CN(C)c1cc(N2CCOC2=O)c2nc(C(=O)NCc3ccc(F)cc3)c(OCc3ccccc3)c(=O)n2c1 ; CN(C)c1cc(N2CCOC2=O)c2nc(C(=O)NCc3ccc(F)cc3)c(O)c(=O)n2c1]

The product from Example 4.2 (60 mg, 0.11 mmol) was dissolved in TFA (5 ml) and heated at 70° C. for 4 h. TFA was removed in vacuo and then methanol (5 ml) was added. The solids were collected by filtration, washed with methanol and dried in vacuo to afford the desired compound (30 mg, 60%).

¹H NMR (300 MHz, DMSO-d⁶) δ 12.05 (s, 1H), 9.31 (t, J=3.0 Hz, 1H), 7.90 (d, J=2.7 Hz, 1H), 7.81 (d, J=2.4 Hz, 1H), 7.45-7.33 (m, 2H), 7.17 (t, J=8.7 Hz, 2H), 4.59-4.51 (m, 4H), 4.19 (t, J=8.0 Hz, 2H), 2.98 (s, 6H).

MS (ESI⁺) m/z 440 (M−1)

### Example 8

### Preparation of 7-(dimethylamino)-N-(4-fluorobenzyl)-3-hydroxy-4-oxo-9-(2-oxo-1,3-oxazinan-3-yl)-4H-pyrido[1,2-a]pyrimidine-2-carboxamide

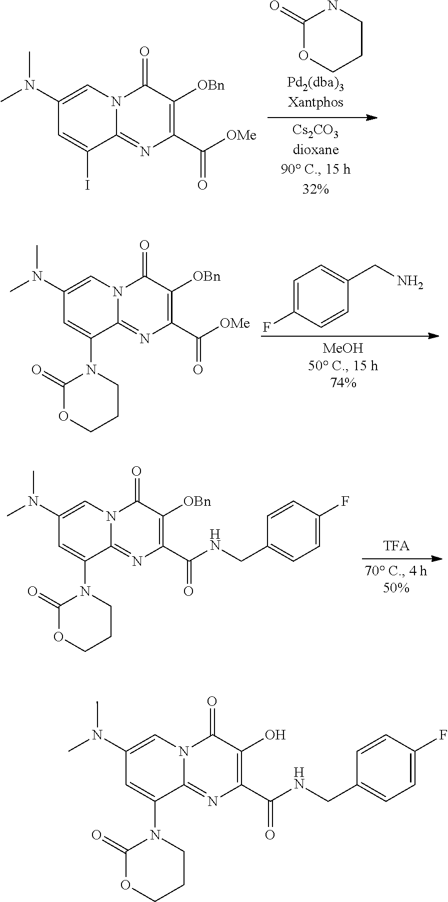

This target was made by adapting the procedure described in Example 7.

### Example 8.1

### Preparation of methyl 3-(benzyloxy)-7-(dimethylamino)-4-oxo-9-(2-oxo-1,3-oxazinan-3-yl)-4H-pyrido[1,2-a]pyrimidine-2-carboxylate

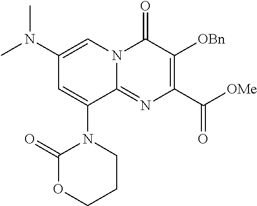 [SMILES: COC(=O)c1nc2c(N3CCCOC3=O)cc(N(C)C)cn2c(=O)c1OCc1ccccc1]

This compound was made by adapting the procedure described in example 7.1.

¹H NMR (300 MHz, CDCl₃) δ 8.15 (d, J=2.7 Hz, 1H), 7.62 (d, J=2.7 Hz, 1H), 7.57-7.51 (m, 2H), 7.43-7.31 (m, 3H), 5.29 (s, 2H), 4.53 (t, J=2.7 Hz, 2H), 3.89 (s, 3H), 3.84-3.71 (m, 2H), 3.07 (s, 6H), 2.35-2.24 (m, 2H).

MS (ESI⁺) m/z 453 (M+1), 475 (M+23)

### Example 8.2

### Preparation of 3-(benzyloxy)-7-(dimethylamino)-N-(4-fluorobenzyl)-4-oxo-9-(2-oxo-1,3-oxazinan-3-yl)-4H-pyrido[1,2-a]pyrimidine-2-carboxamide

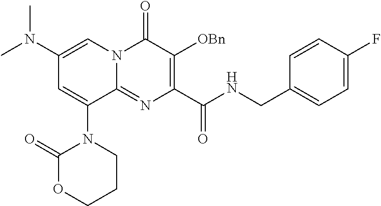 [SMILES: CN(C)c1cc(N2CCCOC2=O)c2nc(C(=O)NCc3ccc(F)cc3)c(OCc3ccccc3)c(=O)n2c1]

This compound was made by adapting the procedure described in example 7.2.

¹H NMR (300 MHz, CDCl₃) δ 8.11 (d, J=2.7 Hz, 1H), 7.63-7.55 (m, 2H), 7.54-7.48 (m, 2H), 7.38-7.27 (m, 5H), 7.01 (t, J=8.7 Hz, 2H), 5.31 (s, 2H), 4.54 (d, J=5.1 Hz, 2H), 4.32 (t, J=4.8 Hz, 2H), 3.78-3.66 (m, 2H), 3.06 (s, 6H), 2.18-2.07 (m, 2H).

MS (ESI⁺) m/z 546 (M+1), 568 (M+23)

### Example 8.3

### Preparation of 7-(dimethylamino)-N-(4-fluorobenzyl)-3-hydroxy-4-oxo-9-(2-oxo-1,3-oxazinan-3-yl)-4H-pyrido[1,2-a]pyrimidine-2-carboxamide

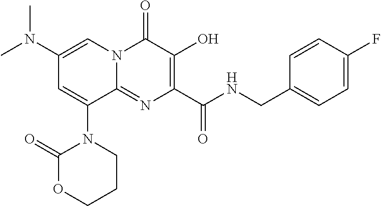 [SMILES: CN(C)c1cc(N2CCCOC2=O)c2nc(C(=O)NCc3ccc(F)cc3)c(O)c(=O)n2c1]

This compound was made by adapting the procedure described in example 7.3.

¹H NMR (300 MHz, DMSO-d⁶) δ 11.88 (s, 1H), 8.89 (t, J=6.6 Hz, 1H), 7.95 (d, J=2.7 Hz, 1H), 7.81 (d, J=2.7 Hz, 1H), 7.47-7.33 (m, 2H), 7.19 (t, J=8.7 Hz, 2H), 4.57 (d, J=6.6 Hz, 2H), 4.40 (t, J=4.5 Hz, 2H), 3.94-3.47 (m, 2H), 2.99 (s, 6H), 2.22-2.07 (m, 2H).

MS (ESI⁺) m/z 456 (M+1), 478 (M+23)

### Example 9

### Preparation of 7-(dimethylamino)-N-(4-fluorobenzyl)-3-hydroxy-4-oxo-9-(2-oxopiperazin-1-yl)-4H-pyrido[1,2-a]pyrimidine-2-carboxamide

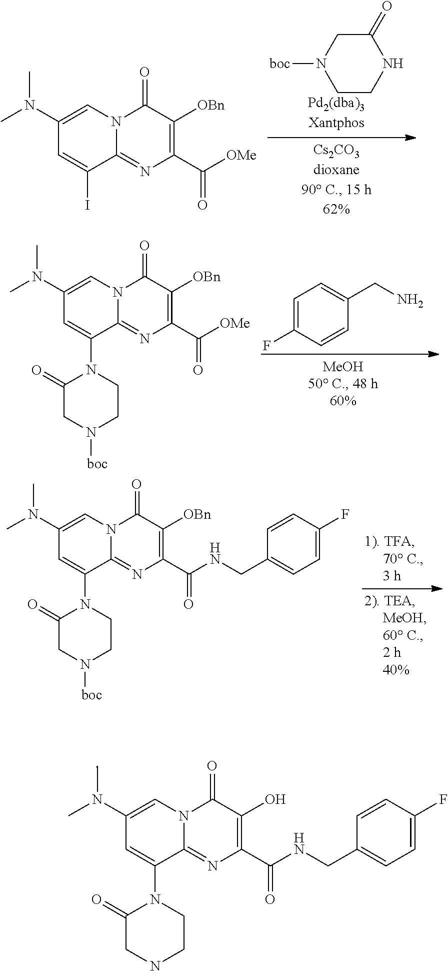

This target was made by adapting the procedure described in the Example 7, except that TEA was used for desalting of the deprotection product.

### Example 9.1

### Preparation of methyl 3-(benzyloxy)-9-(4-(tert-butoxycarbonyl)-2-oxopiperazin-1-yl)-7-(dimethylamino)-4-oxo-4H-pyrido[1,2-a]pyrimidine-2-carboxylate

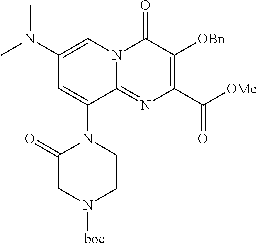

This compound was made by adapting the procedure described in example 7.1.

¹H NMR (300 MHz, CDCl₃) δ 8.17 (d, J=2.7 Hz, 1H), 7.60-7.50 (m, 3H), 7.42-7.36 (m, 3H), 5.29 (s, 2H), 4.32 (s, 2H), 3.92-3.80 (m, 7H), 3.07 (s, 6H), 1.50 (s, 9H).

MS (ESI⁺) m/z 552 (M+1), 574 (M+23)

### Example 9.2

### Preparation of tert-butyl 4-(3-(benzyloxy)-7-(dimethylamino)-2-(4-fluorobenzylcarbamoyl)-4-oxo-4H-pyrido[1,2-a]pyrimidin-9-yl)-3-oxopiperazine-1-carboxylate

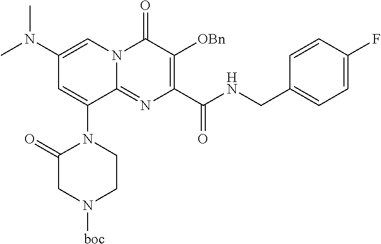

This compound was made by adapting the procedure described in example 7.2.

¹H NMR (300 MHz, CDCl₃) δ 8.14 (d, J=2.7 Hz, 1H), 7.63 (t, J=4.8 Hz, 1H), 7.56 (d, J=2.7 Hz, 1H), 7.49-7.46 (m, 2H), 7.34-7.30 (m, 3H), 7.27-7.22 (m, 2H), 7.00 (t, J=8.7 Hz, 2H), 5.30 (s, 2H), 4.51 (d, J=5.4 Hz, 2H), 4.23 (s, 2H), 3.84-3.73 (m, 4H), 3.08 (s, 6H), 1.50 (s, 9H).

MS (ESI⁺) m/z 645 (M+1), 667 (M+23)

### Example 9.3

### Preparation of 7-(dimethylamino)-N-(4-fluorobenzyl)-3-hydroxy-4-oxo-9-(2-oxopiperazin-1-yl)-4H-pyrido[1,2-a]pyrimidine-2-carboxamide

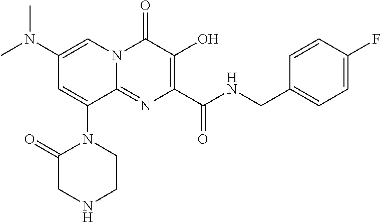 [SMILES: CN(C)c1cc(N2CCNCC2=O)c2nc(C(=O)NCc3ccc(F)cc3)c(O)c(=O)n2c1]

This compound was made by adapting the procedure described in example 7.3, except that TEA was used for desalting of the deprotection product.

¹H NMR (300 MHz, DMSO-d⁶) δ 8.84 (t, J=4.8 Hz, 1H), 7.80 (m, 2H), 7.40 (m, 2H), 7.19 (t, J=8.7 Hz, 2H), 4.58 (d, J=6 Hz, 2H), 3.78-3.50 (m, 2H), 3.41 (s, 2H), 3.09-3.01 (m, 2H), 2.98 (s, 6H).

MS (ESI⁺) m/z 455 (M+1)

### Example 10

### Preparation of N-(4-fluorobenzyl)-3-hydroxy-7-morpholino-4-oxo-9-(2-oxopiperazin-1-yl)-4H-pyrido[1,2-a]pyrimidine-2-carboxamide

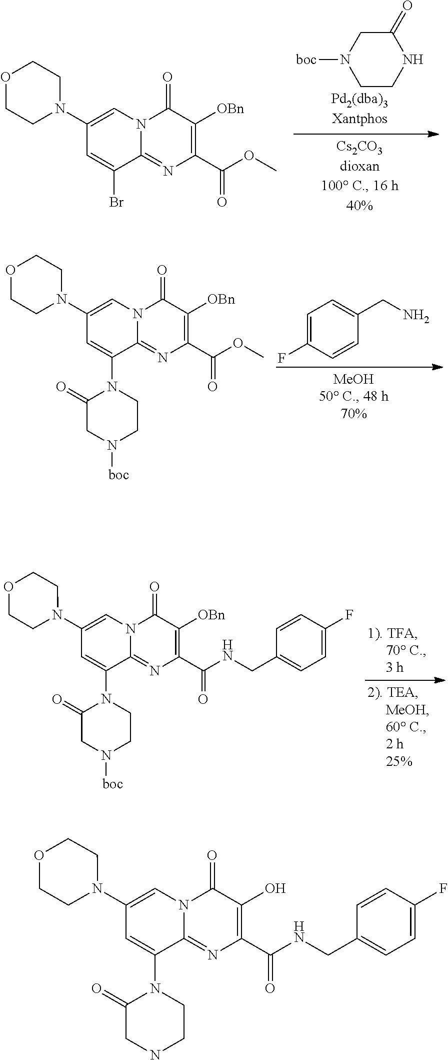

This target was made by adapting the procedure described in the Example 7, except that TEA was used for desalting of the deprotection product.

### Example 10.1

### Preparation of 3-Benzyloxy-9-(4-tert-butoxycarbonyl-2-oxo-piperazin-1-yl)-7-morpholin-4-yl-4-oxo-4H-pyrido[1,2-a]pyrimidine-2-carboxylic acid methyl ester

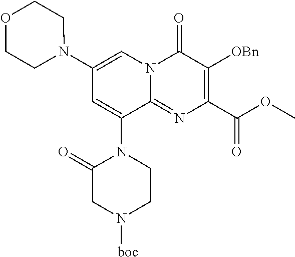

This compound was made by adapting the procedure described in example 7.1.

¹H NMR (300 MHz, CDCl₃) δ 8.32 (d, J=2.6 Hz, 1H), 7.62 (d, J=2.6 Hz, 1H), 7.56-7.47 (m, 2H), 7.42-7.32 (m, 3H), 5.30 (s, 2H), 4.31 (s, 2H), 3.96-3.74 (m, 11H), 3.30-3.18 (m, 4H), 1.51 (s, 9H).

MS (ESI⁺) m/z 594 (M+1)

### Example 10.2

### Preparation of tert-butyl 4-(2-(4-fluorobenzylcarbamoyl)-3-hydroxy-7-morpholino-4-oxo-4H-pyrido[1,2-a]pyrimidin-9-yl)-3-oxopiperazine-1-carboxylate

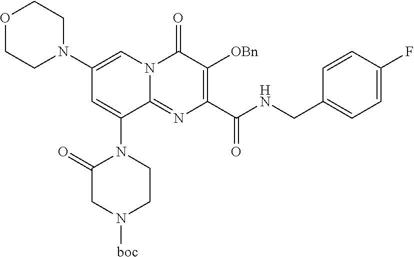

This compound was made by adapting the procedure described in example 7.2.

¹H NMR (300 MHz, CDCl₃) δ 8.28 (d, J=2.4 Hz, 1H), 7.64-7.56 (m, 2H), 7.48-7.44 (m, 2H), 7.36-7.31 (m, 3H), 7.28-7.22 (m, 2H), 7.00 (t, J=8.7 Hz, 2H), 5.30 (s, 2H), 4.51 (d, J=5.4 Hz, 2H), 4.22 (s, 2H), 3.93-3.74 (m, 8H), 3.23 (t, J=4.3 Hz, 4H), 1.50 (s, 9H).

MS (ESI⁺) m/z 687 (M+1), 709 (M+23)

### Example 10.3

### Preparation of N-(4-fluorobenzyl)-3-hydroxy-7-morpholino-4-oxo-9-(2-oxopiperazin-1-yl)-4H-pyrido[1,2-a]pyrimidine-2-carboxamide

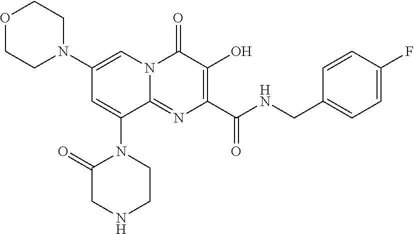 [SMILES: O=C(NCc1ccc(F)cc1)c1nc2c(N3CCNCC3=O)cc(N3CCOCC3)cn2c(=O)c1O]

This compound was made by adapting the procedure described in example 7.3, except that TEA was used for desalting of the deprotection product.

¹H NMR (300 MHz, DMSO-d⁶) δ 8.90 (t, J=6.0 Hz, 1H), 7.96 (d, J=1.8 Hz, 1H), 7.91 (d, J=1.8 Hz, 1H), 7.46-7.34 (m, 2H), 7.19 (t, J=8.7 Hz, 2H), 4.58 (d, J=6 Hz, 2H), 3.76 (t, J=4.3 Hz, 4H), 3.67-3.53 (m, 2H), 3.42 (s, 2H), 3.18 (t, J=4.3 Hz, 4H), 3.09-2.99 (m, 2H).

MS (ESI⁺) m/z 497 (M+1), 519 (M+23)

### Example 11

### Preparation of N-(4-fluorobenzyl)-3-hydroxy-7-(3-methylmorpholino)-4-oxo-9-(2-oxopiperazin-1-yl)-4H-pyrido[1,2-a]pyrimidine-2-carboxamide

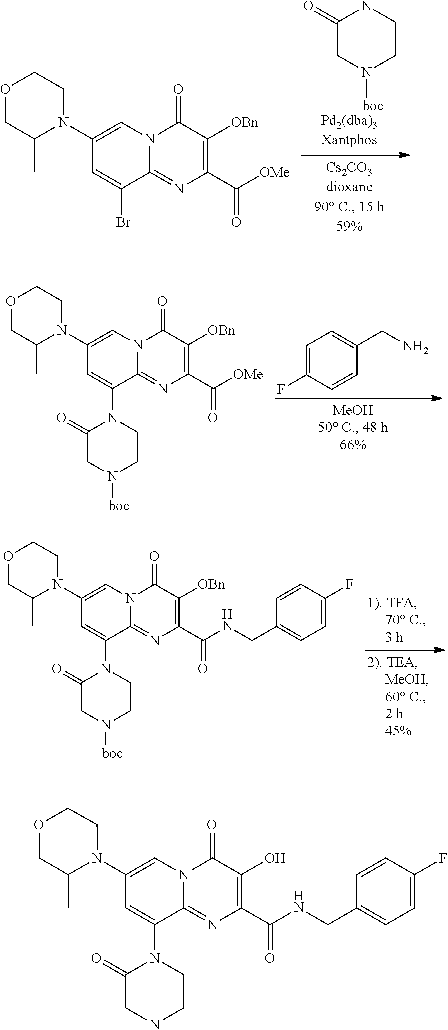

This target was made by adapting the procedure described in the Example 7, except that TEA was used for desalting of the deprotection product.

The starting material 7-(3-methylmorpholino)-9-Br core was made in a similar method to its morpholine derivative. 3-Methylmorpholine was made according to US2005/38032 A1.

### Example 11.1

### Preparation of methyl 3-(benzyloxy)-9-(4-(tert-butoxycarbonyl)-2-oxopiperazin-1-yl)-7-(3-methylmorpholino)-4-oxo-4H-pyrido[1,2-a]pyrimidine-2-carboxylate

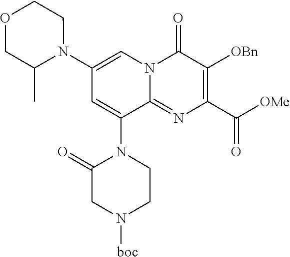

This compound was made by adapting the procedure described in example 7.1.

¹H NMR (300 MHz, CDCl₃) δ 8.28 (d, J=2.7 Hz, 1H), 7.61 (d, J⁼2.7 Hz, 1H), 7.56-7.52 (m, 2H), 7.43-7.30 (m, 3H), 5.30 (s, 2H), 4.31 (s, 2H), 4.04 (d, J=10.2 Hz, 1H), 3.92-3.63 (m, 11H), 3.38-3.26 (m, 1H), 3.22-3.14 (m, 1H), 1.51 (s, 9H), 1.22 (d, J=6.3 Hz, 3H).

MS (ESI⁺) m/z 608 (M+1), 630 (M+23)

### Example 11.2

### Preparation of tert-butyl 4-(3-(benzyloxy)-2-(4-fluorobenzyl carbamoyl)-7-(3-methylmorpholino)-4-oxo-4H-pyrido[1,2-a]pyrimidin-9-yl)-3-oxopiperazine-1-carboxylate

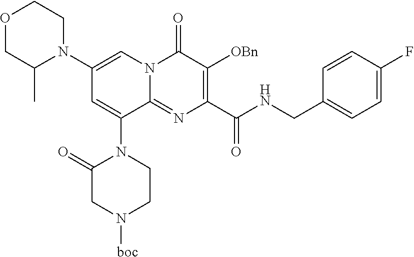

This compound was made by adapting the procedure described in example 7.2.

¹H NMR (300 MHz, CDCl₃) δ 8.23 (d, J=2.4 Hz, 1H), 7.63-7.55 (m, 2H), 7.50-7.44 (m, 2H), 7.37-7.30 (m, 3H), 7.30-7.22 (m, 2H), 7.00 (t, J=8.7 Hz, 2H), 5.30 (s, 2H), 4.51 (d, J=6 Hz, 2H), 4.22 (s, 2H), 4.02 (d, J=9.9 Hz, 1H), 3.65-3.90 (m, 8H), 3.37-3.25 (m, 1H), 3.21-3.13 (m, 1H), 1.50 (s, 9H), 1.22 (d, J=6.6 Hz, 3H).

MS (ESI⁺) m/z 701 (M+1), 723 (M+23)

### Example 11.3

### Preparation of N-(4-fluorobenzyl)-3-hydroxy-7-(3-methylmorpholino)-4-oxo-9-(2-oxopiperazin-1-yl)-4H-pyrido[1,2-a]pyrimidine-2-carboxamide

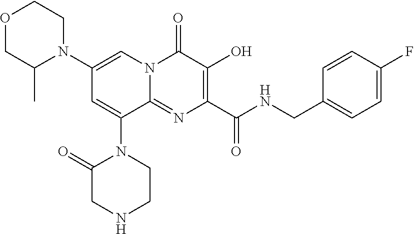 [SMILES: CC1COCCN1c1cc(N2CCNCC2=O)c2nc(C(=O)NCc3ccc(F)cc3)c(O)c(=O)n2c1]

This compound was made by adapting the procedure described in example 7.3, except that TEA was used for desalting of the deprotection product.

¹H NMR (300 MHz, DMSO-d⁶) δ 8.89 (t, J=6.0 Hz, 1H), 7.93-7.91 (m, 2H), 7.46-7.33 (m, 2H), 7.19 (t, J=8.7 Hz, 2H), 4.58 (d, J=6.0 Hz, 2H), 3.96 (d, J=8.8 Hz, 2H), 3.72 (s, 2H), 3.65-3.51 (m, 3H), 3.44-3.40 (m, 2H), 3.24-2.96 (m, 4H), 1.06 (d, J=6.5 Hz, 3H).

MS (ESI⁺) m/z 511 (M+1)

### Example 12

### Preparation of 3-Hydroxy-4-oxo-9-(2-oxo-piperazin-1-yl)-7-piperidin-1-yl-4H-pyrido[1,2-a]pyrimidine-2-carboxylic acid 4-fluoro-benzylamide

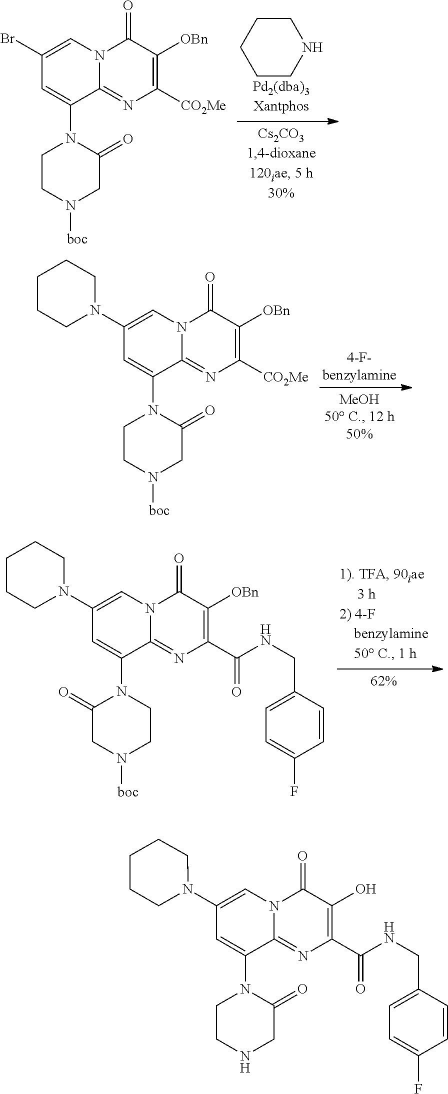

This target was prepared by adapting the procedure described in Example 4.

### Example 12.1

### Preparation of 3-Benzyloxy-9-(4-tert-butoxycarbonyl-2-oxo-piperazin-1-yl)-4-oxo-7-piperidin-1-yl-4H-pyrido[1,2-a]pyrimidine-2-carboxylic acid methyl ester

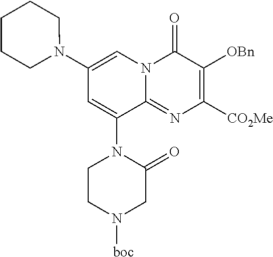

This compound was made by adapting the procedure described in example 4.2.

¹H NMR (300 MHz, CDCl₃) δ 8.31 (d, J=2.7 Hz, 1H), 7.65 (d, J=2.7 Hz, 1H), 7.57-7.47 (m, 2H), 7.45-7.30 (m, 3H), 5.32 (s, 2H), 4.32 (s, 2H), 3.94-3.72 (m, 7H), 3.28-3.18 (m, 4H), 1.82-1.54 (m, 6H), 1.52 (s, 9H).

MS (ESI⁺) m/z 614 (M+23)

### Example 12.2

### Preparation of 4-[3-Benzyloxy-2-(4-fluoro-benzylcarbamoyl)-4-oxo-7-piperidin-1-yl-4H-pyrido[1,2-a]pyrimidin-9-yl]-3-oxo-piperazine-1-carboxylic acid tert-butyl ester

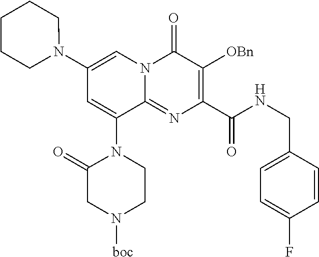

This compound was made by adapting the procedure described in example 4.3.

¹H NMR (300 MHz, CDCl₃) δ 8.27 (d, J=2.6 Hz, 1H), 7.65-7.54 (m, 2H), 7.52-7.42 (m, 2H), 7.36-7.28 (m, 3H), 7.26-7.20 (m, 2H), 7.00 (t, J=8.6 Hz, 2H), 5.32 (s, 2H), 4.52 (d, J=6.0 Hz, 2H), 4.23 (s, 2H), 3.85-3.65 (m, 4H), 3.28-3.18 (m, 4H), 1.80-1.60 (m, 6H), 1.51 (s, 9H).

MS (ESI⁺) m/z 685 (M+1)

### Example 12.3

### Preparation of 3-Hydroxy-4-oxo-9-(2-oxo-piperazin-1-yl)-7-piperidin-1-yl-4H-pyrido[1,2-a]pyrimidine-2-carboxylic acid 4-fluoro-benzylamide

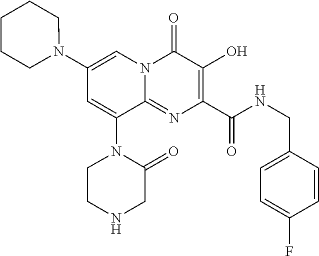 [SMILES: O=C(NCc1ccc(F)cc1)c1nc2c(N3CCNCC3=O)cc(N3CCCCC3)cn2c(=O)c1O]

This compound was made by adapting the procedure described in example 4.4.

¹H NMR (300 MHz, CDCl₃) δ 8.25-8.10 (m, 2H), 7.46 (d, J=2.4 Hz, 1H), 7.33 (dd, J=6.0, 8.5 Hz, 2H), 7.06 (t, J=8.1 Hz, 2H), 4.56 (d, J=5.1 Hz, 2H), 3.78-3.40 (m, 4H), 3.25-2.90 (m, 6H), 1.85-1.50 (m, 6H).

MS (ESI⁺) m/z 495 (M+1)

### Example 13

### Preparation of 7-(Cyclopropylmethyl-methyl-amino)-3-hydroxy-4-oxo-9-(2-oxo-piperazin-1-yl)-4H-pyrido[1,2-a]pyrimidine-2-carboxylic acid 4-fluoro-benzylamide

This target was made by adapting the procedure described in Example 4, except that an extra step of reduction amination step was required.

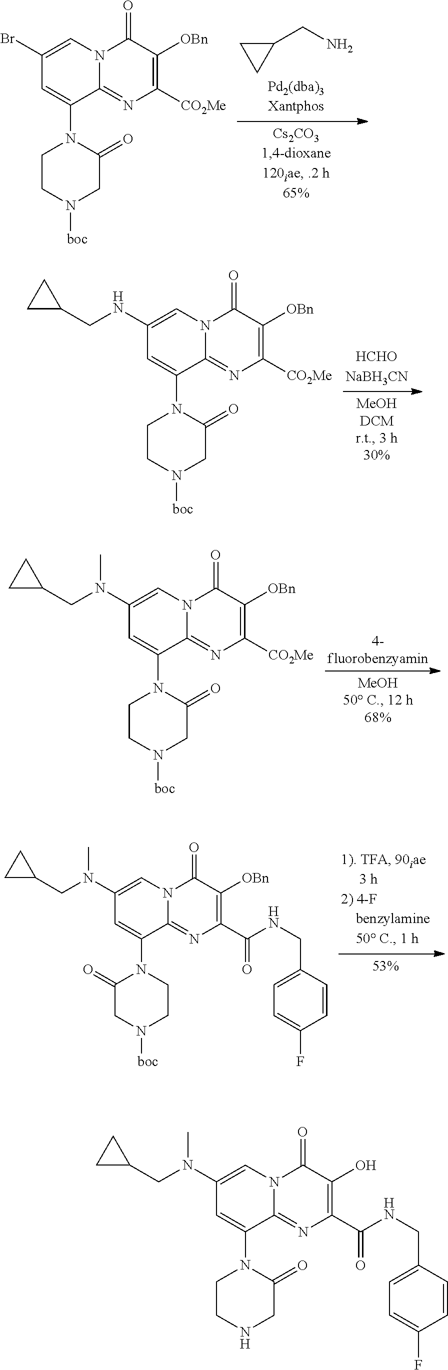

### Example 13.1

### Preparation of 3-Benzyloxy-9-(4-tert-butoxycarbonyl-2-oxo-piperazin-1-yl)-7-(cyclopropylmethyl-amino)-4-oxo-4H-pyrido[1,2-a]pyrimidine-2-carboxylic acid methyl ester

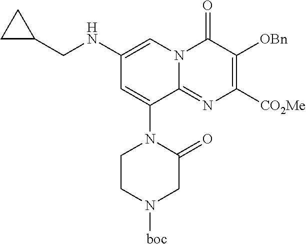

This compound was made by adapting the procedure described in example 4.2.

¹H NMR (300 MHz, CDCl₃) δ 8.06 (d, J=2.0 Hz, 1H), 7.58-7.48 (m, 2H), 7.42-7.28 (m, 5H), 5.32 (s, 2H), 4.32 (s, 2H), 3.95-3.70 (m, 7H), 2.95 (s, 2H), 1.50 (s, 9H), 1.25-1.15 (m, 1H), 0.68-0.54 (m, 2H), 0.32-0.22 (m, 2H).

MS (ESI⁺) m/z 578 (M+1)

### Example 13.2

### Preparation of 3-Benzyloxy-9-(4-tert-butoxycarbonyl-2-oxo-piperazin-1-yl)-7-(cyclopropylmethyl-methyl-amino)-4-oxo-4H-pyrido[1,2-a]pyrimidine-2-carboxylic acid methyl ester

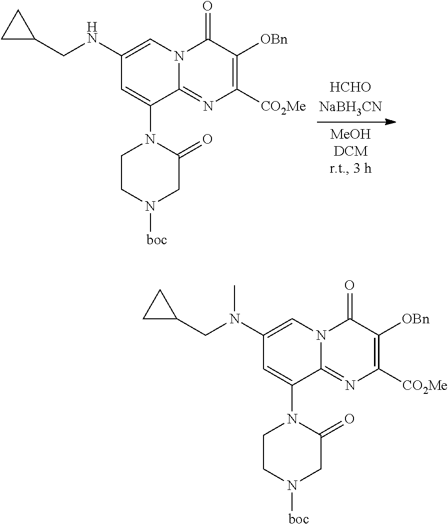

The product from example 13.1 (450 mg, 0.781 mmol) was suspended in a mixed solvent of DCM (2 ml) and MeOH (2 ml). To the above stirred mixture was added HCHO (100 mg, 3.30 mmol), HOAc (201), NaBH₃CN (200 mg 3.2 mmol) successively. The mixture was stirred at room temperature for 30 minutes, after which it was concentrated in vacuo. The residue was purified on column chromatography (PE/EtOAc=1/1) to give the crude product, which was further purified on preparative TLC to afford the desired product (130 mg, 30%).

¹H NMR (300 MHz, CDCl₃) δ 8.21 (d, J=2.6 Hz, 1H), 7.60 (d, J=2.6 Hz, 1H), 7.56-7.48 (m, 2H), 7.41-7.30 (m, 3H), 5.32 (s, 2H), 4.32 (s, 2H), 3.93-3.77 (m, 7H), 3.29 (d, J=6.8 Hz, 2H), 3.10 (s, 3H), 1.50 (s, 9H), 1.25-1.15 (m, 1H), 0.75-0.65 (m, 2H), 0.33-0.23 (m, 2H).

MS (ESI⁺) m/z 592 (M+1)

### Example 13.3

### Preparation of 4-[3-Benzyloxy-7-(cyclopropylmethyl-methyl-amino)-2-(4-fluoro-benzylcarbamoyl)-4-oxo-4H-pyrido[1,2-a]pyrimidin-9-yl]-3-oxo-piperazine-1-carboxylic acid tert-butyl ester

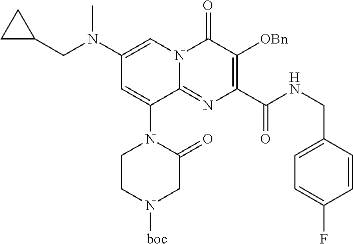

This compound was made by adapting the procedure described in example 4.3.

¹H NMR (300 MHz, CDCl₃) δ 8.17 (d, J=2.6 Hz, 1H), 7.67-7.55 (m, 2H), 7.54-7.42 (m, 2H), 7.38-7.29 (m, 3H), 7.26-7.18 (m, 2H), 7.00 (t, J=8.5 Hz, 2H), 5.32 (s, 2H), 4.52 (d, J=5.4 Hz, 2H), 4.22 (s, 2H), 3.85-3.70 (m, 4H), 3.29 (d, J=6.6 Hz, 2H), 3.08 (s, 3H), 1.50 (s, 9H), 1.10-1.00 (m, 1H), 0.69-0.55 (m, 2H), 0.30-0.20 (m, 2H).

MS (ESI⁺) m/z 685 (M+1)

### Example 13.4

### Preparation of 7-(Cyclopropylmethyl-methyl-amino)-3-hydroxy-4-oxo-9-(2-oxo-piperazin-1-yl)-4H-pyrido[1,2-a]pyrimidine-2-carboxylic acid 4-fluoro-benzylamide

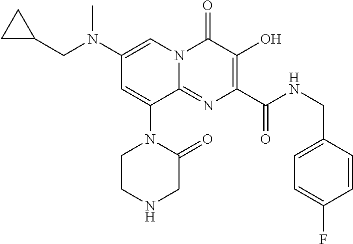 [SMILES: CN(CC1CC1)c1cc(N2CCNCC2=O)c2nc(C(=O)NCc3ccc(F)cc3)c(O)c(=O)n2c1]

This compound was made by adapting the procedure described in example 4.4.

¹H NMR (300 MHz, CDCl₃) δ 8.14 (t, J=4.8 Hz, 1H), 8.03 (d, J=2.4 Hz, 1H), 7.46 (d, J=2.4 Hz, 1H), 7.33 (dd, J=5.9, 8.2 Hz, 2H), 7.07 (t, J=8.2 Hz, 2H), 4.60 (d, J=5.1 Hz, 2H), 3.80-3.35 (m, 4H), 3.24 (d, J=6.3 Hz, 2H), 3.15-2.95 (m, 5H), 1.10-0.95 (m, 1H), 0.65-0.50 (m, 2H), 0.30-0.18 (m, 2H).

MS (ESI⁺) m/z 495 (M+1)

### Example 14

### Preparation of 3-Hydroxy-7-(4-methyl-piperazin-1-yl)-4-oxo-9-(2-oxo-piperazin-1-yl)-4H-pyrido[1,2-a]pyrimidine-2-carboxylic acid 4-fluoro-benzylamide

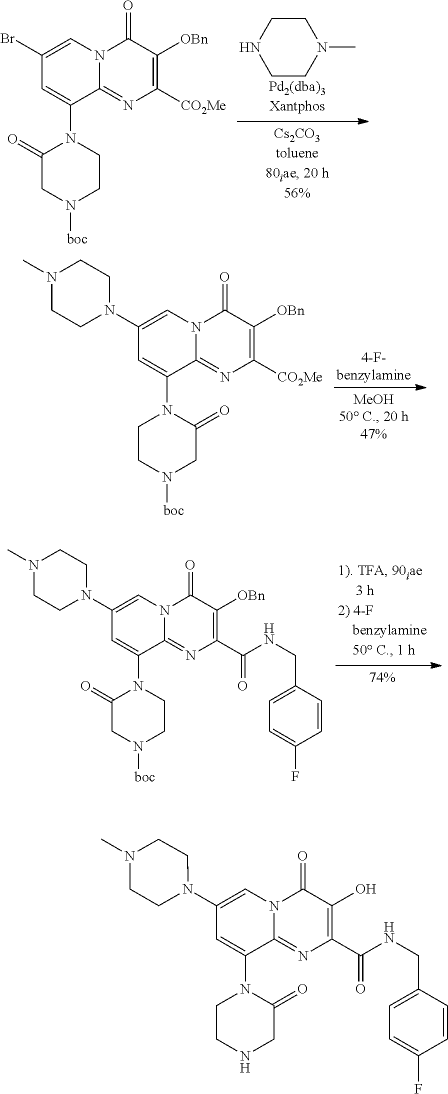

This target was made by adapting the procedure described in Example 4.

### Example 14.1

### Preparation of 3-Benzyloxy-9-(4-tert-butoxycarbonyl-2-oxo-piperazin-1-yl)-7-(4-methyl-piperazin-1-yl)-4-oxo-4H-pyrido[1,2-a]pyrimidine-2-carboxylic acid methyl ester

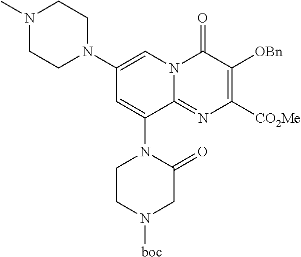

This compound was made by adapting the procedure described in example 4.2.

¹H NMR (300 MHz, CDCl₃) δ 8.33 (d, J=2.4 Hz, 1H), 7.76 (d, J=2.4 Hz, 1H), 7.58-7.48 (m, 2H), 7.47-7.37 (m, 3H), 5.32 (s, 2H), 4.31 (s, 2H), 3.95-3.75 (m, 7H), 3.36-3.24 (m, 4H), 2.68-2.57 (m, 4H), 2.38 (s, 3H), 1.53 (s, 9H).

MS (ESI⁺) m/z 607 (M+1)

### Example 14.2

### Preparation of 4-[3-Benzyloxy-2-(4-fluoro-benzylcarbamoyl)-7-(4-methyl-piperazin-1-yl)-4-oxo-4H-pyrido[1,2-a]pyrimidin-9-yl]-3-oxo-piperazine-1-carboxylic acid tert-butyl ester

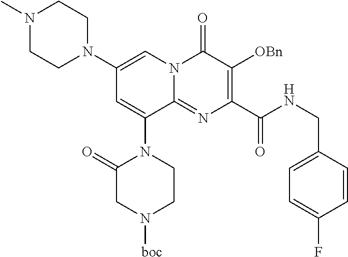

This compound was made by adapting the procedure described in example 4.3 and the crude product was used in the next step.

MS (ESI⁺) m/z 700 (M+1).

### Example 14.3

### Preparation of 3-Hydroxy-7-(4-methyl-piperazin-1-yl)-4-oxo-9-(2-oxo-piperazin-1-yl)-4H-pyrido[1,2-a]pyrimidine-2-carboxylic acid 4-fluoro-benzylamide

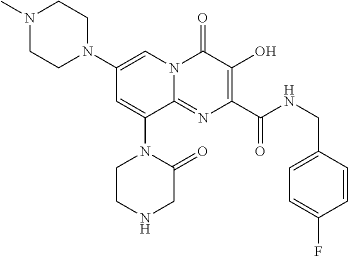 [SMILES: CN1CCN(c2cc(N3CCNCC3=O)c3nc(C(=O)NCc4ccc(F)cc4)c(O)c(=O)n3c2)CC1]

This compound was made by adapting the procedure described in example 4.4.

¹H NMR (300 MHz, CDCl₃) δ 8.20-8.02 (m, 2H), 7.48 (d, J=2.1 Hz, 1H), 7.34 (dd, J=4.9, 8.1 Hz, 2H), 7.07 (t, J=8.5 Hz, 2H), 4.59 (d, J=5.1 Hz, 2H), 3.80-3.40 (m, 4H), 3.30-3.16 (m, 4H), 3.08 (s, 2H), 2.68-2.50 (m, 4H), 2.36 (s, 3H).

MS (ESI⁺) m/z 510 (M+1)

### Example 15

### Preparation of 3-Hydroxy-4-oxo-9-(2-oxo-piperazin-1-yl)-7-piperazin-1-yl-4H-pyrido[1,2-a]pyrimidine-2-carboxylic acid 4-fluoro-benzylamide

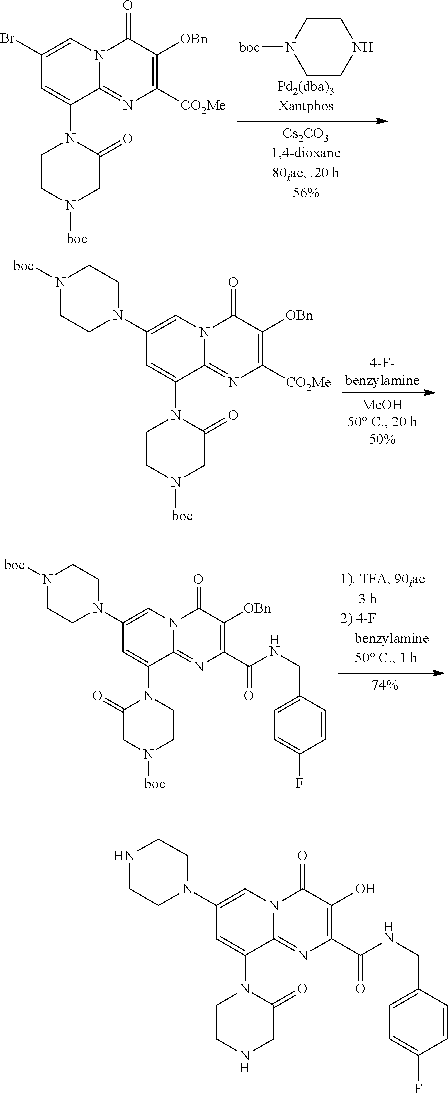

This target was prepared by adapting the procedure described in Example 4.

### Example 15.1

### Preparation of 3-Benzyloxy-9-(4-tert-butoxycarbonyl-2-oxo-piperazin-1-yl)-7-(4-tert-butoxycarbonyl-piperazin-1-yl)-4-oxo-4H-pyrido[1,2-a]pyrimidine-2-carboxylic acid methyl ester

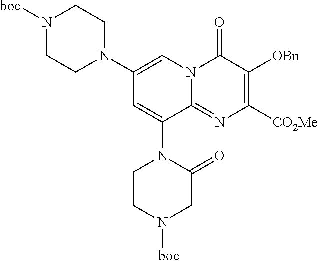

This compound was made by adapting the procedure described in example 4.2.

¹H NMR (300 MHz, CDCl₃) δ 8.34 (d, J=2.1 Hz, 1H), 7.64 (d, J=2.1 Hz, 1H), 7.55-7.47 (m, 2H), 7.43-7.30 (m, 3H), 5.32 (s, 2H), 4.32 (s, 2H), 3.92-3.75 (m, 7H), 3.67-3.55 (m, 4H), 3.26-3.16 (m, 4H), 1.50 (d, J=4.2 Hz, 18H).

MS (ESI⁺) m/z 693 (M+1)

### Example 15.2

### Preparation of 4-[3-Benzyloxy-7-(4-tert-butoxycarbonyl-piperazin-1-yl)-2-(4-fluoro-benzylcarbamoyl)-4-oxo-4H-pyrido[1,2-a]pyrimidin-9-yl]-3-oxo-piperazine-1-carboxylic acid tert-butyl ester

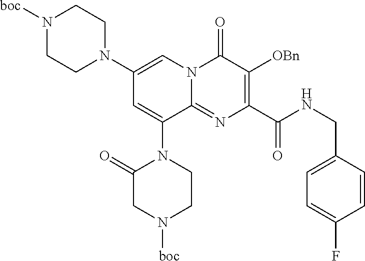

This compound was made by adapting the procedure described in example 4.3.

¹H NMR (300 MHz, CDCl₃) δ 8.27 (d, J=2.4 Hz, 1H), 7.67-7.52 (m, 2H), 7.50-7.38 (m, 2H), 7.37-7.28 (m, 3H), 7.27-7.17 (m, 2H), 7.00 (t, J=8.7 Hz, 2H), 5.32 (s, 2H), 4.52 (d, J=5.1 Hz, 2H), 4.23 (s, 2H), 3.84-3.70 (m, 4H), 3.66-3.54 (m, 4H), 3.28-3.14 (m, 4H), 1.49 (d, J=3.1 Hz, 18H)

MS (ESI⁺) m/z 786 (M+1)

### Example 15.3

### Preparation of 3-Hydroxy-4-oxo-9-(2-oxo-piperazin-1-yl)-7-piperazin-1-yl-4H-pyrido[1,2-a]pyrimidine-2-carboxylic acid 4-fluoro-benzylamide

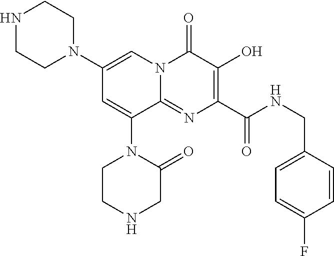 [SMILES: O=C(NCc1ccc(F)cc1)c1nc2c(N3CCNCC3=O)cc(N3CCNCC3)cn2c(=O)c1O]

This compound was made by adapting the procedure described in example 4.4.

¹H NMR (300 MHz, CDCl₃) δ 8.23-8.07 (m, 2H), 7.45 (d, J=2.7 Hz, 1H), 7.40-7.30 (m, 2H), 7.07 (t, J=8.3 Hz, 2H), 4.59 (d, J=5.5 Hz, 2H), 3.80-3.40 (m, 4H), 3.25-3.12 (m, 4H), 3.12-2.95 (m, 6H).

MS (ESI⁺) m/z 496 (M+1)

### Example 16

### Preparation of 3-Hydroxy-9-(4-methyl-2-oxo-piperazin-1-yl)-7-morpholin-4-yl-4-oxo-4H-pyrido[1,2-a]pyrimidine-2-carboxylic acid 4-fluoro-benzylamide

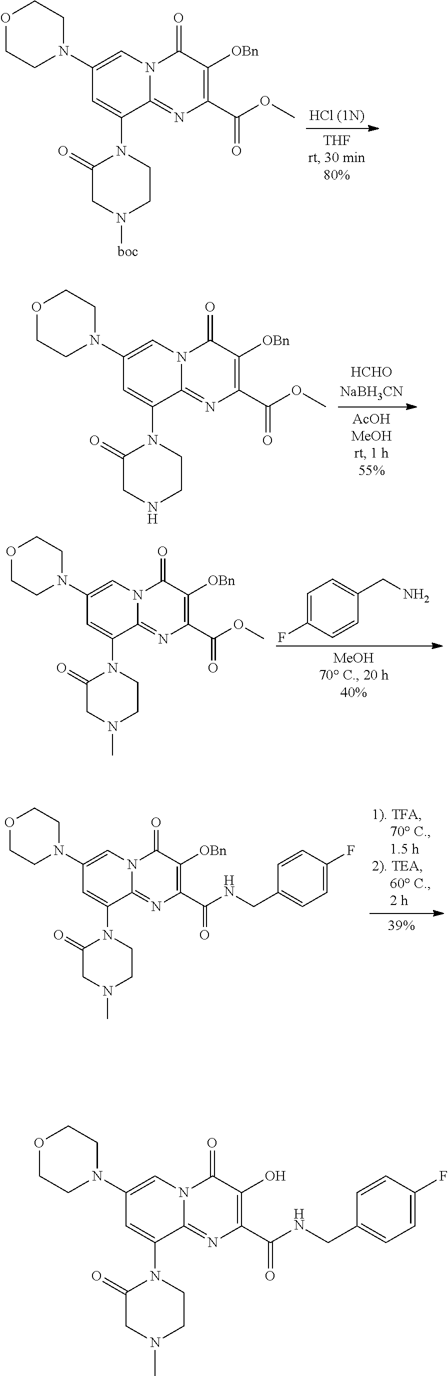

This target was made by adapting the procedure described in Example 7, except that TEA was used for desalting of the final target, and an extra step was required for the reductive amination.

### Example 16.1

### Preparation of 3-Benzyloxy-7-morpholin-4-yl-4-oxo-9-(2-oxo-piperazin-1-yl)-4H-pyrido[1,2-a]pyrimidine-2-carboxylic acid methyl ester

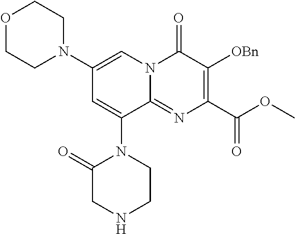 [SMILES: COC(=O)c1nc2c(N3CCNCC3=O)cc(N3CCOCC3)cn2c(=O)c1OCc1ccccc1]

This compound was made by adapting the procedure described in example 7.1.

¹H NMR (300 MHz, CDCl₃) δ 8.32 (d, J=2.5 Hz, 1H), 7.64 (d, J=2.5 Hz, 1H), 7.56-7.47 (m, 2H), 7.42-7.30 (m, 3H), 5.29 (s, 2H), 3.96-3.74 (m, 11H), 3.34 (t, J=5.1 Hz, 2H), 3.28-3.16 (m, 4H).

MS (ESI⁺) m/z 494 (M+1)

### Example 16.2

### Preparation of 3-Benzyloxy-9-(4-methyl-2-oxo-piperazin-1-yl)-7-morpholin-4-yl-4-oxo-4H-pyrido[1,2-a]pyrimidine-2-carboxylic acid methyl ester

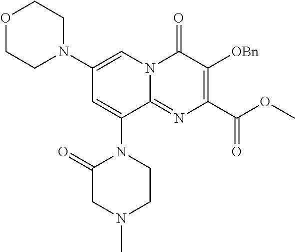 [SMILES: COC(=O)c1nc2c(N3CCN(C)CC3=O)cc(N3CCOCC3)cn2c(=O)c1OCc1ccccc1]

This compound was made by adapting the procedure described in example 13.2.

¹H NMR (300 MHz, CDCl₃) δ 8.32 (d, J=2.4 Hz, 1H), 7.67 (d, J=2.5 Hz, 1H), 7.56-7.45 (m, 2H), 7.43-7.31 (m, 3H), 5.43-5.15 (m, 2H), 4.35 (s, 2H), 4.01-3.41 (m, 11H), 3.26 (t, J=4.5 Hz, 4H), 3.14 (s, 3H).

MS (ESI⁺) m/z 508 (M+1)

### Example 16.3

### Preparation of 3-Benzyloxy-9-(4-methyl-2-oxo-piperazin-1-yl)-7-morpholin-4-yl-4-oxo-4H-pyrido[1,2-a]pyrimidine-2-carboxylic acid 4-fluoro-benzylamide

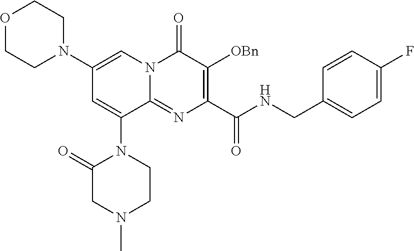 [SMILES: CN1CCN(c2cc(N3CCOCC3)cn3c(=O)c(OCc4ccccc4)c(C(=O)NCc4ccc(F)cc4)nc23)C(=O)C1]

This compound was made by adapting the procedure described in example 7.2.

¹H NMR (300 MHz, CDCl₃) δ 8.27 (d, J=2.8 Hz, 1H), 7.80-7.68 (m, 1H), 7.64-7.50 (m, 3H), 7.42-7.31 (m, 5H), 7.03 (t, J=8.7 Hz, 2H), 5.31 (s, 2H), 4.55 (d, J=5.6 Hz, 2H), 3.88 (t, J=4.6 Hz, 4H), 3.80-3.66 (brs, 2H), 3.30-3.10 (m, 6H), 2.77-2.60 (brs, 2H), 2.31 (s, 3H).

MS (ESI⁺) m/z 601 (M+1)

### Example 16.4

### Preparation of 3-Hydroxy-9-(4-methyl-2-oxo-piperazin-1-yl)-7-morpholin-4-yl-4-oxo-4H-pyrido[1,2-a]pyrimidine-2-carboxylic acid 4-fluoro-benzylamide

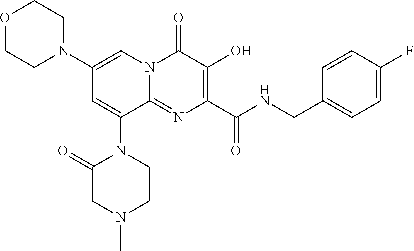 [SMILES: CN1CCN(c2cc(N3CCOCC3)cn3c(=O)c(O)c(C(=O)NCc4ccc(F)cc4)nc23)C(=O)C1]

This compound was made by adapting the procedure described in example 7.3, except that TEA was used for desalting.

¹H NMR (300 MHz, DMSO-d⁶) δ 11.80-11.60 (brs, 1H), 8.54 (t, J=5.6 Hz, 1H), 8.02-7.94 (m, 2H), 7.44 (dd, J=5.8, 8.2 Hz, 2H), 7.22 (t, J=8.8 Hz, 2H), 4.57 (d, J=5.6 Hz, 2H), 3.82-3.70 (m, 4H), 3.66-3.50 (m, 2H), 3.25-3.14 (m, 4H), 3.09 (s, 2H), 2.76-2.62 (m, 2H), 2.22 (s, 3H).

MS (ESI⁺) m/z 511 (M+1)

HPLC: 98.9%

### Example 17

### Preparation of 3-Hydroxy-9-(3-isopropyl-2-oxo-imidazolidin-1-yl)-7-(4-methyl-piperazin-1-yl)-4-oxo-4H-pyrido[1,2-a]pyrimidine-2-carboxylic acid 4-fluoro-benzylamide

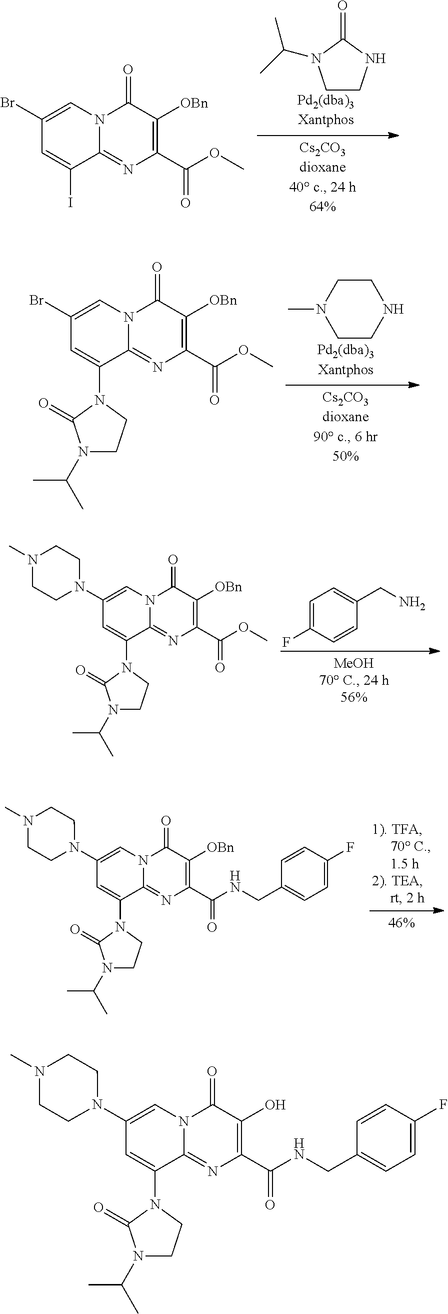

This target was made by adapting the procedure described in the Example 4, except that TEA was used in the desalting step.

### Example 17.1

### Preparation of 3-Benzyloxy-7-bromo-9-(3-isopropyl-2-oxo-imidazolidin-1-yl)-4-oxo-4H-pyrido[1,2-a]pyrimidine-2-carboxylic acid methyl ester

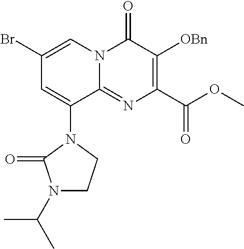 [SMILES: COC(=O)c1nc2c(N3CCN(C(C)C)C3=O)cc(Br)cn2c(=O)c1OCc1ccccc1]

This compound was made by adapting the procedure described in example 4.1.

¹H NMR (300 MHz, CDCl₃) δ 8.95 (d, J=1.9 Hz, 1H), 8.14 (d, J=1.9 Hz, 1H), 7.44-7.54 (m, 2H), 7.30-7.42 (m, 3H), 5.34 (s, 2H), 4.20-4.40 (m, 3H), 3.90 (s, 3H), 3.48 (t, J=7.9 Hz, 2H), 1.21 (d, J=6.8 Hz, 6H).

MS (ESI⁺) m/z 537 (M[Br⁷⁹]+23), 539 (M[Br⁸¹]+23)

### Example 17.2

### Preparation of 3-Benzyloxy-9-(3-isopropyl-2-oxo-imidazolidin-1-yl)-7-(4-methyl-piperazin-1-yl)-4-oxo-4H-pyrido[1,2-a]pyrimidine-2-carboxylic acid methyl ester

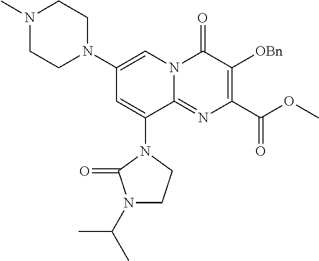

This compound was made by adapting the procedure described in example 4.2.

¹H NMR (300 MHz, CDCl₃) δ 8.24 (d, J=2.5 Hz, 1H), 8.01 (d, J=2.5 Hz, 1H), 7.48-7.58 (m, 2H), 7.28-7.42 (m, 3H), 5.31 (s, 2H), 4.39-4.16 (m, 3H), 3.89 (s, 3H), 3.48 (t, J=8.1 Hz, 2H), 3.24-3.36 (m, 4H), 2.54-2.68 (m, 4H), 2.38 (s, 3H), 1.21 (d, J=6.7 Hz, 6H).

MS (ESI⁺) m/z 535 (M+1)

### Example 17.3

### Preparation of 3-Benzyloxy-9-(3-isopropyl-2-oxo-imidazolidin-1-yl)-7-(4-methyl-piperazin-1-yl)-4-oxo-4H-pyrido[1,2-a]pyrimidine-2-carboxylic acid 4-fluoro-benzylamide

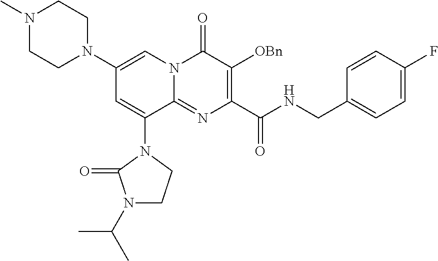 [SMILES: CC(C)N1CCN(c2cc(N3CCN(C)CC3)cn3c(=O)c(OCc4ccccc4)c(C(=O)NCc4ccc(F)cc4)nc23)C1=O]

This compound was made by adapting the procedure described in example 4.3.

¹H NMR (300 MHz, DMSO-d⁶) δ 8.93 (t, J=6.1 Hz, 1H), 8.13 (d, J=1.9 Hz, 1H), 7.98 (d, J=1.9 Hz, 1H), 7.52-7.42 (m, 2H), 7.41-7.30 (m, 5H), 7.07 (t, J^{=8.8} Hz, 2H), 5.14 (s, 2H), 4.45 (d, J=5.9 Hz, 2H), 4.16-3.93 (m, 3H), 3.42 (t, J=7.8 Hz, 2H), 3.30-3.12 (m, 4H), 2.58-2.41 (m, 4H), 2.24 (s, 3H), 1.13 (d, J=6.7 Hz, 6H).

MS (ESI⁺) m/z 650 (M+23)

### Example 17.4

### Preparation of 3-Hydroxy-9-(3-isopropyl-2-oxo-imidazolidin-1-yl)-7-(4-methyl-piperazin-1-yl)-4-oxo-4H-pyrido[1,2-a]pyrimidine-2-carboxylic acid 4-fluoro-benzylamide

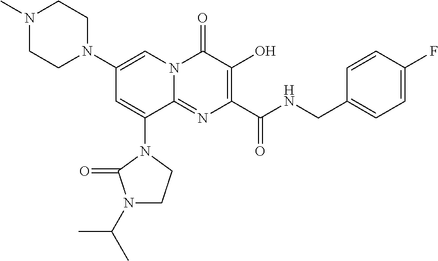 [SMILES: CC(C)N1CCN(c2cc(N3CCN(C)CC3)cn3c(=O)c(O)c(C(=O)NCc4ccc(F)cc4)nc23)C1=O]

This compound was made by adapting the procedure described in example 4.4, except that TEA was used in desalting.

¹H NMR (300 MHz, CDCl₃) δ 11.83-11.65 (m, 1H), 8.13 (d, J=2.5 Hz, 1H), 7.94 (t, J=5.6 Hz, 1H), 7.61 (d, J=2.5 Hz, 1H), 7.38-7.29 (m, 2H), 7.07 (t, J=8.6 Hz, 2H), 4.62 (d, J=5.7 Hz, 2H), 4.24-4.08 (m, 1H), 3.85 (t, J=7.8 Hz, 2H), 3.33 (m, J=7.8 Hz, 2H), 3.29-3.19 (m, 4H), 2.66-2.50 (m, 4H), 2.36 (s, 3H), 1.11 (d, J^{=6.8} Hz, 6H).

MS (ESI⁺) m/z 560 (M+23)

HPLC: 99.1%

### Example 18

### Preparation of 3-Hydroxy-7-(4-methyl-piperazin-1-yl)-4-oxo-9-(2-oxo-piperidin-1-yl)-4H-pyrido[1,2-a]pyrimidine-2-carboxylic acid 4-fluoro-benzylamide

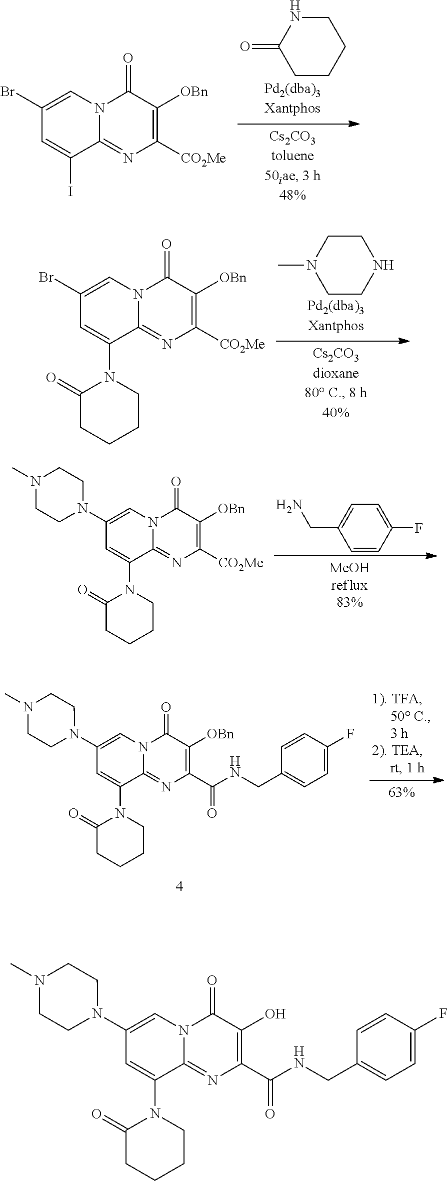

This target was made by adapting the procedure described in Example 4, except that TEA was used instead of 4-fluorobenzylamine in the desalting step.

### Example 18.1

### Preparation of 3-Benzyloxy-7-bromo-4-oxo-9-(2-oxo-piperidin-1-yl)-4H-Pyrido[1,2-a]pyrimidine-2-carboxylic acid methyl ester

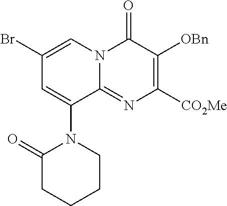 [SMILES: COC(=O)c1nc2c(N3CCCCC3=O)cc(Br)cn2c(=O)c1OCc1ccccc1]

This compound was made by adapting the procedure described in example 4.1.

¹H NMR (300 MHz, CDCl₃) δ 9.06 (d, J=1.8 Hz, 1H), 7.67 (d, J=2.1 Hz, 1H), 7.53-7.45 (m, 2H), 7.41-7.31 (m, 3H), 5.31 (s, 2H), 3.88 (s, 3H), 3.75-3.60 (brs, 2H), 2.64-2.53 (m, 2H), 2.06-1.93 (m, 4H)

MS (ESI⁺) m/z 486 (M+1), 508 (M+23)

### Example 18.2

### Preparation of 3-Benzyloxy-7-(4-methyl-piperazin-1-yl)-4-oxo-9-(2-oxo-piperidin-1-yl)-4H-pyrido[1,2-a]pyrimidine-2-carboxylic acid methyl ester

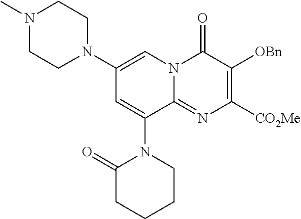 [SMILES: COC(=O)c1nc2c(N3CCCCC3=O)cc(N3CCN(C)CC3)cn2c(=O)c1OCc1ccccc1]

This compound was made by adapting the procedure described in example 4.2.

¹H NMR (300 MHz, CDCl₃) δ 8.30 (d, J=2.1 Hz, 1H), 7.59 (d, J=2.4 Hz, 1H), 7.53-7.49 (m, 2H), 7.41-7.30 (m, 3H), 5.28 (s, 2H), 3.87 (s, 3H), 3.79-3.61 (m, 2H), 3.33-3.24 (m, 4H), 2.65-2.54 (m, 6H), 2.37 (s, 3H), 2.06-1.93 (m, 4H)

MS (ESI⁺) m/z 506 (M+1), 528 (M+23)

### Example 18.3

### Preparation of 3-Benzyloxy-7-(4-methyl-piperazin-1-yl)-4-oxo-9-(2-oxo-piperidin-1-yl)-4H-pyrido[1,2-a]pyrimidine-2-carboxylic acid 4-fluoro-benzylamide

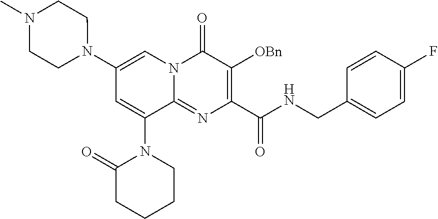 [SMILES: CN1CCN(c2cc(N3CCCCC3=O)c3nc(C(=O)NCc4ccc(F)cc4)c(OCc4ccccc4)c(=O)n3c2)CC1]

This compound was made by adapting the procedure described in example 4.3.

¹H NMR (300 MHz, DMSO-d⁶) δ 8.26 (d, J=2.7 Hz, 1H), 7.49-7.43 (m, 4H), 7.40-7.31 (m, 5H), 7.13-7.04 (m, 2H), 5.13 (s, 2H), 4.48-4.41 (m, 2H), 3.74-3.46 (m, 2H), 3.37-3.41 (m, 4H), 2.50-2.70 (m, 4H), 2.30-2.50 (m, 5H), 1.89-1.77 (m, 4H)

MS (ESI⁺) m/z 599 (M+1)

### Example 18.4

### Preparation of 3-Hydroxy-7-(4-methyl-piperazin-1-yl)-4-oxo-9-(2-oxo-piperidin-1-yl)-4H-pyrido[1,2-a]pyrimidine-2-carboxylic acid 4-fluoro-benzylamide

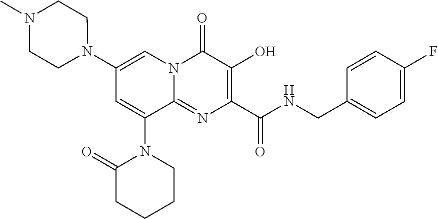 [SMILES: CN1CCN(c2cc(N3CCCCC3=O)c3nc(C(=O)NCc4ccc(F)cc4)c(O)c(=O)n3c2)CC1]

This compound was made by adapting the procedure described in example 4.4, except that TEA was used instead of 4-fluorobenzylamine in the desalting step.

¹H NMR (300 MHz, CDCl₃) δ 11.79 (s, 1H), 8.15 (d, J=1.8 Hz, 1H), 7.83 (m, 1H), 7.42 (d, J=1.8, 1H), 7.39-7.30 (m, 2H), 7.13-7.03 (m, 2H), 4.68-4.47 (m, 2H), 3.70-3.80 (m, 2H), 3.29-3.18 (m, 4H), 2.64-2.52 (m, 4H), 2.50-2.30 (m, 5H), 1.94-1.51 (m, 4H)

MS (ESI⁺) m/z 509 (M+1)

### Example 19

### Preparation of 3-Hydroxy-4-oxo-9-(2-oxo-oxazolidin-3-yl)-7-piperazin-1-yl-4H-pyrido[1,2-a]pyrimidine-2-carboxylic acid 4-fluoro-benzylamide

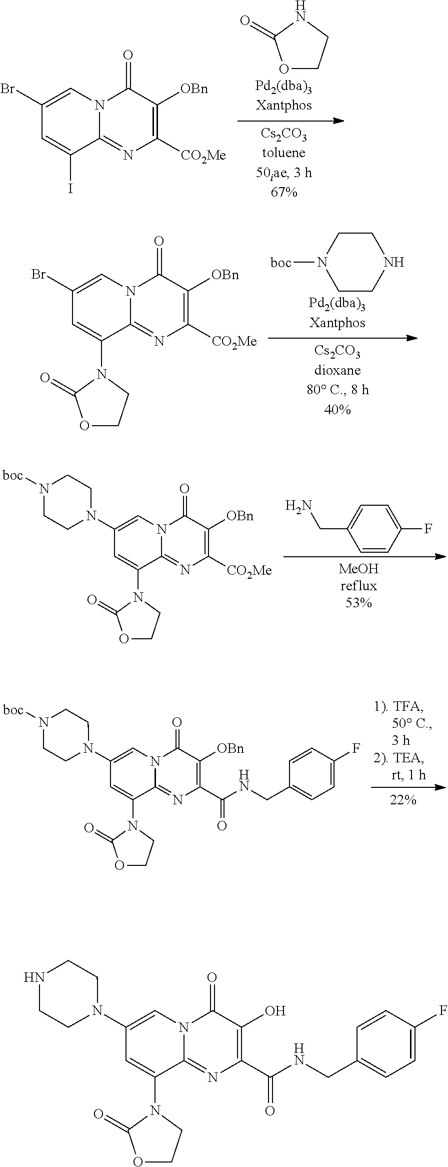

This target was made by adapting the procedure described in Example 4, except that TEA was used instead of 4-fluorobenzylamine in the desalting step.

### Example 19.1

### Preparation of 3-Benzyloxy-7-bromo-4-oxo-9-(2-oxo-oxazolidin-3-yl)-4H-pyrido[1,2-a]pyrimidine-2-carboxylic acid methyl ester

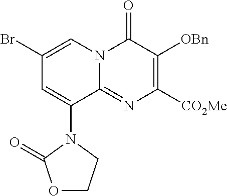 [SMILES: COC(=O)c1nc2c(N3CCOC3=O)cc(Br)cn2c(=O)c1OCc1ccccc1]

This compound was made by adapting the procedure described in example 4.1.

¹H NMR (300 MHz, CDCl₃) δ 9.07 (d, J=1.8 Hz, 1H), 7.67 (d, J=2.4 Hz, 1H), 7.53-7.45 (m, 2H), 7.41-7.31 (m, 3H), 5.31 (s, 2H), 3.88 (s, 3H), 3.75-3.62 (m, 2H), 2.63-2.55 (m, 2H)

MS (ESI⁺) m/z 474 (M+1)

### Example 19.2

### Preparation of 3-Benzyloxy-7-(4-tert-butoxycarbonyl-piperazin-1-yl)-4-oxo-9-(2-oxo-oxazolidin-3-yl)-4H-pyrido[1,2-a]pyrimidine-2-carboxylic acid methyl ester

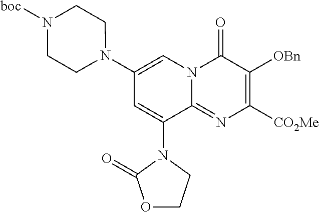

This compound was made by adapting the procedure described in example 4.2.

¹H NMR (300 MHz, CDCl₃) δ 8.28 (d, J=2.7 Hz, 1H), 7.91 (d, J=2.4 Hz, 1H), 7.52-7.47 (m, 2H), 7.40-7.31 (m, 3H), 5.31 (s, 2H), 4.57-4.50 (m, 4H), 3.89 (s, 3H), 3.65-3.57 (m, 4H), 3.25-3.17 (m, 4H), 1.49 (s, 9H)

MS (ESI⁺) m/z 580 (M+1), 602 (M+23)

### Example 19.3

### Preparation of 4-[3-Benzyloxy-2-(4-fluoro-benzylcarbamoyl)-4-oxo-9-(2-oxo-oxazolidin-3-yl)-4H-pyrido[1,2-a]pyrimidin-7-yl]-piperazine-1-carboxylic acid tert-butyl ester

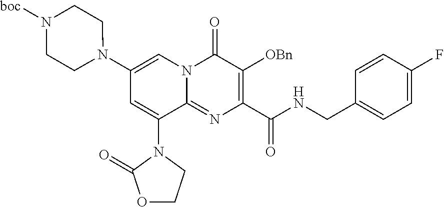

This compound was made by adapting the procedure described in example 4.3.

¹H NMR (300 MHz, DMSO-d₆) δ 8.99 (t, J=6.0 Hz, 1H), 8.21 (d, J=2.1 Hz, 1H), 8.17 (d, J=2.1 Hz, 1H), 7.49-7.41 (m, 2H), 7.41-7.32 (m, 5H), 7.07 (t, J=8.7 Hz, 2H), 5.14 (s, 2H), 4.56-4.42 (m, 4H), 4.26-4.16 (m, 2H), 3.56-3.47 (m, 4H), 3.27-3.19 (m, 4H), 1.45 (s, 9H)

MS (ESI⁺) m/z 673 (M+1), 695 (M+23)

### Example 19.4

### Preparation of 3-Hydroxy-4-oxo-9-(2-oxo-oxazolidin-3-yl)-7-piperazin-1-yl-4H-pyrido[1,2-a]pyrimidine-2-carboxylic acid 4-fluoro-benzylamide

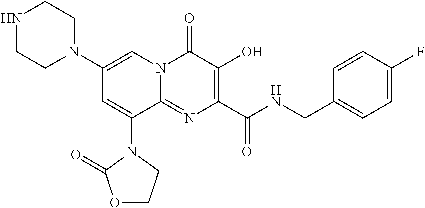 [SMILES: O=C(NCc1ccc(F)cc1)c1nc2c(N3CCOC3=O)cc(N3CCNCC3)cn2c(=O)c1O]

This compound was made by adapting the procedure described in example 4.4, except that TEA was used instead of 4-fluorobenzylamine in the desalting step.

¹H NMR (300 MHz, CDC₃) δ 8.15 (m, 1H), 7.91-7.81 (m, 1H), 7.62 (d, J=2.7 Hz, 1H), 7.39-7.31 (m, 2H), 7.08 (t, J=8.7 Hz, 2H), 4.65-4.59 (m, 2H), 4.44-4.35 (m, 2H), 4.10-4.02 (m, 2H), 3.22-3.14 (m, 4H), 3.09-3.01 (m, 4H)

MS (ESI⁺) m/z 483 (M+1)

### Example 20

### Preparation of 3-Hydroxy-7-(4-methyl-piperazin-1-yl)-4-oxo-9-(2-oxo-oxazolidin-3-yl)-4H-pyrido[1,2-a]pyrimidine-2-carboxylic acid 4-fluoro-benzylamide

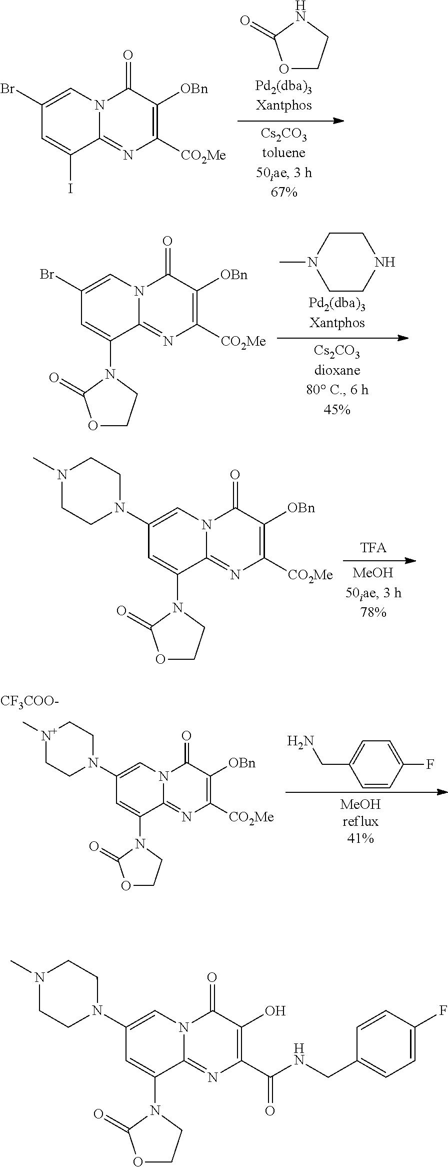

This target was made by adapting the procedure described in Example 2.

### Example 20.1

### Preparation of 3-Benzyloxy-7-bromo-4-oxo-9-(2-oxo-oxazolidin-3-yl)-4H-pyrido[1,2-a]pyrimidine-2-carboxylic acid methyl ester

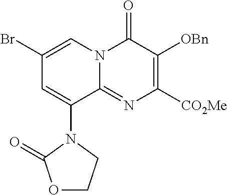 [SMILES: COC(=O)c1nc2c(N3CCOC3=O)cc(Br)cn2c(=O)c1OCc1ccccc1]

This compound was made by adapting the procedure described in example 2.1.

¹H NMR (300 MHz, CDCl₃) δ 9.07 (d, J=1.8 Hz, 1H), 8.05 (d, J=2.4 Hz, 1H), 7.53-7.45 (m, 2H), 7.41-7.31 (m, 3H), 5.31 (s, 2H), 4.43-4.62 (m, 4H), 3.88 (s, 3H)

MS (ESI⁺) no ionisation

### Example 20.2

### Preparation of 3-Benzyloxy-7-(4-methyl-piperazin-1-yl)-4-oxo-9-(2-oxo-Oxazolidin-3-yl)-4H-pyrido[1,2-a]pyrimidine-2-carboxylic acid methyl ester

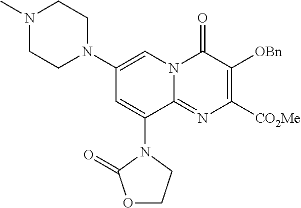 [SMILES: COC(=O)c1nc2c(N3CCOC3=O)cc(N3CCN(C)CC3)cn2c(=O)c1OCc1ccccc1]

This compound was made by adapting the procedure described in example 2.2.

¹H NMR (300 MHz, CDCl₃) δ 8.28 (d, J=2.7 Hz, 1H), 7.93 (d, J=2.7 Hz, 1H), 7.54-7.47 (m, 2H), 7.41-7.31 (m, 3H), 5.31 (s, 2H), 4.60-4.46 (m, 4H), 3.89 (s, 3H), 3.33-3.25 (m, 4H), 2.63-2.55 (m, 4H), 2.37 (s, 3H)

MS (ESI⁺) m/z 494 (M+1), 516 (M+23)

### Example 20.3

### Preparation of Trifluoro-acetate-4-[3-hydroxy-2-methoxy carbonyl-4-oxo-9-(2-oxo-oxazolidin-3-yl)-4H-pyrido[1,2-a]pyrimidin-7-yl]-1-methyl-piperazin-1-ium

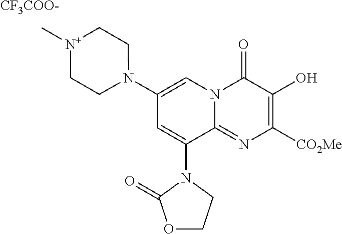 [SMILES: "CF3COO-" ; COC(=O)c1nc2c(N3CCOC3=O)cc(N3CC[NH+](C)CC3)cn2c(=O)c1O]

This compound was made by adapting the procedure described in example 2.3.

¹H NMR (300 MHz, DMSO-d⁶) δ 10.37-10.30 (brs, 1H), 10.05-9.92 (brs, 1H), 8.03 (d, J=2.4 Hz, 1H), 7.97 (d, J=2.4 Hz, 1H), 4.55-4.47 (t, J=7.8 Hz, 2H), 4.28-4.20 (t, J=8.1 Hz, 2H), 3.77-3.98 (m, 5H), 3.69-3.46 (m, 2H), 3.33-2.96 (m, 4H), 2.88 (s, 3H)

MS (ESI⁺) m/z 404 (M-TFA+1)

### Example 20.4

### Preparation of 3-Hydroxy-7-(4-methyl-piperazin-1-yl)-4-oxo-9-(2-oxo-oxazolidin-3-yl)-4H-pyrido[1,2-a]pyrimidine-2-carboxylic acid 4-fluoro-benzylamide

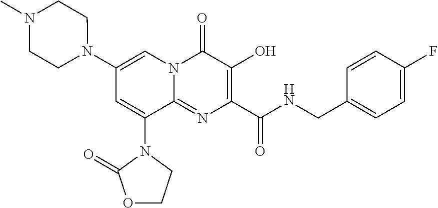 [SMILES: CN1CCN(c2cc(N3CCOC3=O)c3nc(C(=O)NCc4ccc(F)cc4)c(O)c(=O)n3c2)CC1]

This compound was made by adapting the procedure described in example 2.4.

¹H NMR (300 MHz, DMSO-d⁶) δ 12.12-11.69 (brs, 1H), 9.45-9.29 (brs, 1H), 7.98-7.91 (m, 2H), 7.42-7.35 (m, 2H), 7.21-7.11 (m, 2H), 4.58-4.47 (m, 4H), 4.22-4.13 (m, 2H), 3.21-3.14 (m, 4H), 2.60-2.40 (m, 4H), 2.25 (s, 3H)

MS (ESI⁺) m/z 497 (M+1)

### Example 21

### Preparation of N-(4-fluorobenzyl)-3-hydroxy-7,9-dimorpholino-4-oxo-4H-pyrido[1,2-a]pyrimidine-2-carboxamide

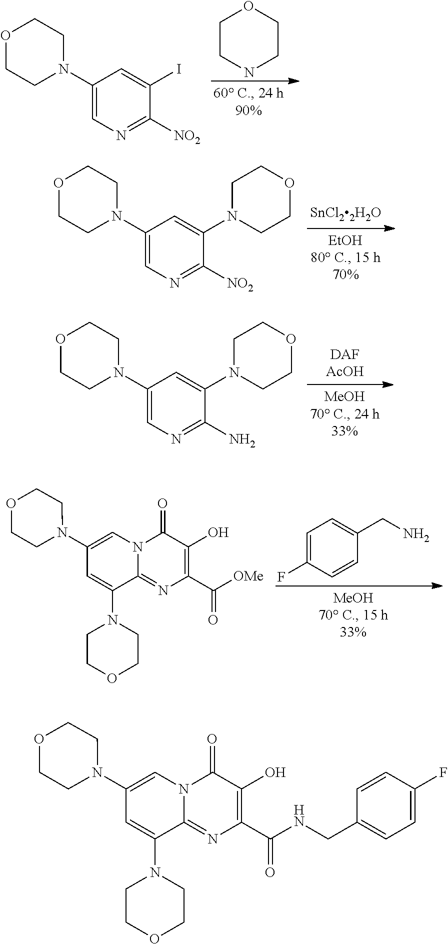

### Example 21.1

### Preparation of 4-(5-iodo-6-nitropyridin-3-yl)morpholine

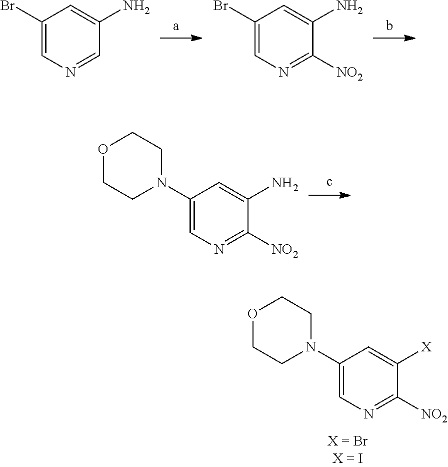

Reagents and conditions: (a) i) ClCOOEt, Py 93% ii) HNO₃, H₂SO₄, 30° C., 20 h, 68% iii) KOH/EtOH/H₂O, 75% (b) morpholine, 140° C., 10 h, 85% (c) NaNO₂, H₂SO₄, H₂O, 0° C., 2 h then 80° C. 2 h, 75% for X═OH or CuBr, X═Br 80% or CuI for X═I, 30%.

### Example 21.2

### Preparation of 4,4′-(2-nitropyridine-3,5-diyl)dimorpholine

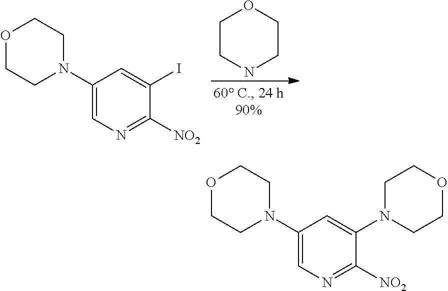

The mixture of 4-(5-iodo-6-nitropyridin-3-yl)morpholine (1.0 g, 3 mmol) and morpholine (10 ml) were heated at 60° C. for 24 h. After cooling down to room temperature, ethyl ether (20 ml) was added. The solids were collected by filtration, washed with water three times and dried in vacuo to afford the desired compound (790 mg, 90%).

¹H NMR (300 MHz, CDCl₃) δ 7.78 (d, J=1.5 Hz, 1H), 6.88 (d, J=1.5 Hz, 1H), 3.96-3.84 (m, 8H), 3.36 (t, J=4.5 Hz, 4H), 3.10 (t, J=4.5 Hz, 4H).

MS (ESI⁺) m/z 295 (M+1)

### Example 21.3

### Preparation of 3,5-dimorpholinopyridin-2-amine

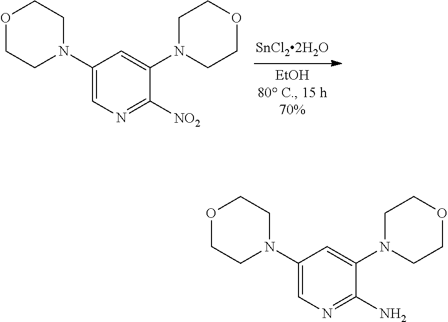 [SMILES: O=[N+]([O-])c1ncc(N2CCOCC2)cc1N1CCOCC1 ; Nc1ncc(N2CCOCC2)cc1N1CCOCC1]

Tin(II) chloride (4.3 g, 18.8 mmol) was added into the solution of the product from Example 21.2 (791 mg, 2.7 mmol) in ethanol (30 ml). The mixture was heated to reflux under nitrogen atmosphere for 15 hours. Then most of ethanol was removed in vacuo and water (30 ml) was added. Aqueous sodium hydroxide (4N) solution was added dropwise to make the pH to ˜9. Then the mixture was extracted with dichloromethane three times. The organic layers were combined, washed with water, dried and evaporated under reduced pressure to afford the desired compound (500 mg, 70%).

¹H NMR (300 MHz, CDCl₃) δ 7.49 (d, J=1.5 Hz, 1H), 6.93 (d, J=1.5 Hz, 1H), 4.60 (s, 2H), 3.91-3.79 (m, 8H), 3.02 (t, J=4.5 Hz, 4H), 2.92 (t, J=4.5 Hz, 4H).

MS (ESI⁺) m/z 265 (M+1)

### Example 21.4

### Preparation of methyl 3-hydroxy-7,9-dimorpholino-4-oxo-4H-pyrido[1,2-a]pyrimidine-2-carboxylate

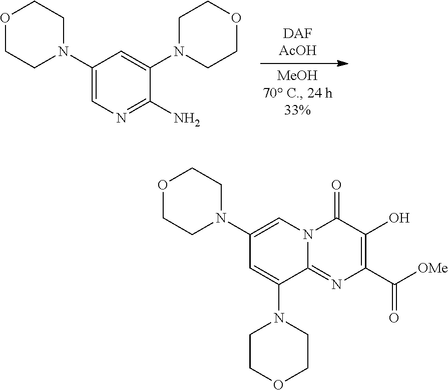 [SMILES: Nc1ncc(N2CCOCC2)cc1N1CCOCC1 ; COC(=O)c1nc2c(N3CCOCC3)cc(N3CCOCC3)cn2c(=O)c1O]

The product from Example 21.3 (250 mg, 0.95 mmol), dimethyl 2,3-diacetoxyfumarate (369 mg, 1.42 mmol) and acetic acid (57 mg, 0.95 mmol) was dissolved in methanol (2 ml) and the mixture was heated at 70° C. for 24 h. After cooling down to room temperature, the solids were collected by filtration, washed with methanol and dried in vacuo to afford the desired compound (123 mg, 33%).

¹H NMR (300 MHz, DMSO-d⁶) δ 10.08 (s, 1H), 7.76 (d, J=1.5 Hz, 1H), 6.96 (d, J=1.5 Hz, 1H), 3.86 (s, 3H), 3.82-3.73 (m, 8H), 3.34 (t, J=4.8 Hz, 4H), 3.17 (t, J=4.8 Hz, 4H).

MS (ESI⁺) m/z 391 (M+1), 413 (M+23), 445 (M+55)

### Example 21.5

### Preparation of N-(4-fluorobenzyl)-3-hydroxy-7,9-dimorpholino-4-oxo-4H-pyrido[1,2-a]pyrimidine-2-carboxamide

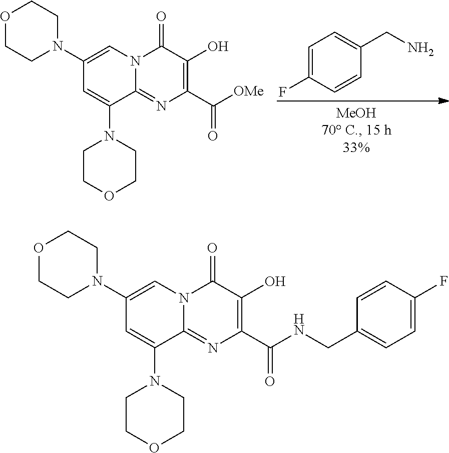

(4-Fluorophenyl)methanamine (64 mg, 0.5 mmol) was added into the solution of the product from Example 21.4 (80 mg, 0.2 mmol) in methanol (5 ml) and the mixture was heated at 70° C. for 15 h. After cooling down to room temperature, the solids were collected by filtration, washed with methanol three times and dried in vacuo to afford the desired compound (33 mg, 33%).

¹H NMR (300 MHz, DMSO-d⁶) δ 11.71 (s, 1H), 8.66 (t, J=6.0 Hz, 1H), 7.79 (d, J=1.5 Hz, 1H), 7.46-7.37 (m, 2H), 7.20 (t, J=8.7 Hz, 2H), 7.02 (d, J=1.5 Hz, 1H), 4.56 (d, J=6.0 Hz, 2H), 3.82-3.71 (m, 8H), 3.24 (t, J=4.2 Hz, 4H), 3.16 (t, J=4.2 Hz, 4H).

MS (ESI⁻) m/z 482 (M−1)

### Example 22

### Preparation of N-(3,4-dichlorobenzyl)-3-hydroxy-7,9-dimorpholino-4-oxo-4H-pyrido[1,2-a]pyrimidine-2-carboxamide

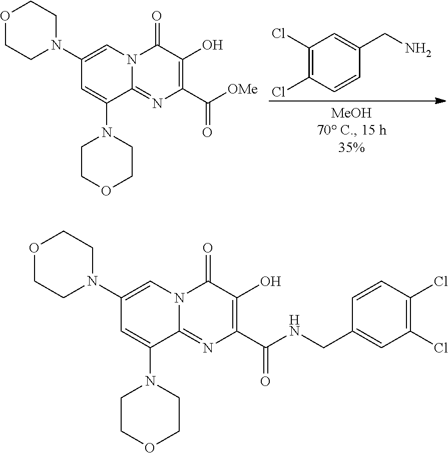 [SMILES: COC(=O)c1nc2c(N3CCOCC3)cc(N3CCOCC3)cn2c(=O)c1O ; NCc1ccc(Cl)c(Cl)c1 ; O=C(NCc1ccc(Cl)c(Cl)c1)c1nc2c(N3CCOCC3)cc(N3CCOCC3)cn2c(=O)c1O]

This compound was prepared by adapting the procedure in Example 21.5.

¹H NMR (300 MHz, DMSO-d⁶) δ 11.60 (s, 1H), 8.80 (t, J=6.0 Hz, 1H), 7.80 (d, J=1.5 Hz, 1H), 7.67-7.61 (m, 2H), 7.39-7.33 (m, 1H), 7.03 (d, J=1.5 Hz, 1H), 4.58 (d, J=6.0 Hz, 2H), 3.81 (t, J=4.5 Hz, 4H), 3.77 (t, J=4.5 Hz, 4H), 3.26 (t, J=4.5 Hz, 4H), 3.16 (t, J=4.5 Hz, 4H).

MS (ESI⁺) m/z 534 (M+1), 556 (M+23)

### Example 23

### Preparation of ethyl 2-(4-fluorobenzylcarbamoyl)-3-hydroxy-7-morpholino-4-oxo-4,9a-dihydro-3H-pyrido[1,2-a]pyrimidin-9-ylcarbamate

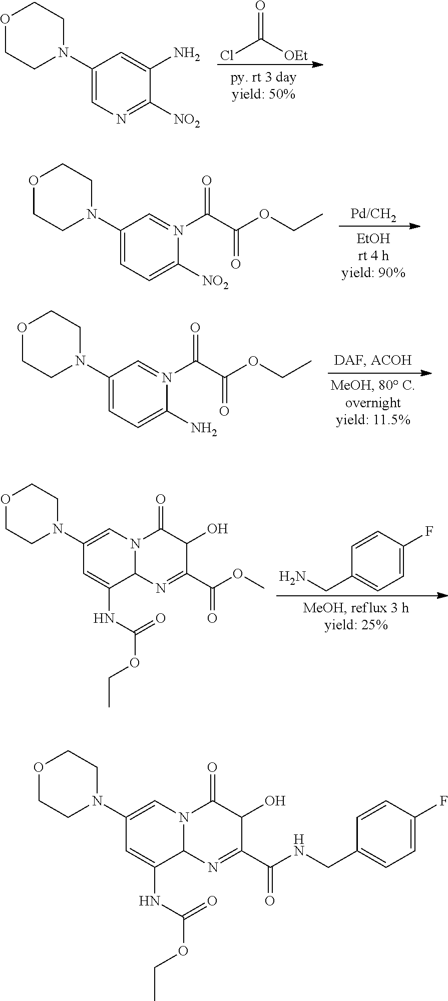

5-Morpholino-2-nitropyridin-3-amine was prepared as described in example 21.1.

### Example 23.1

### Preparation of ethyl 5-morpholino-2-nitropyridin-3-yl carbamate

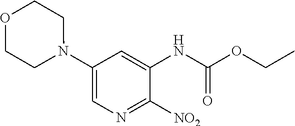 [SMILES: CCOC(=O)Nc1cc(N2CCOCC2)cnc1[N+](=O)[O-]]

To the solution of 5-morpholino-2-nitropyridin-3-amine (2 g, 8.8 mmol) in pyridine (30 ml) was added ethyl carbonochloridate (1.1 ml, 11 mmol) at 0° C. After stirring at room temperature for 3 days, EA was added. The organic phase was washed with saturated aqueous NaCl three times, dried over Na₂SO₄ and concentrated in vacuo. The residue was purified by column chromatography (EA/PE=1/1) to give the desired product (1.2 g, 50%)

¹H NMR (300 M Hz, CDCl₃) δ 10.06 (s, 1H), 8.40 (d, J=2.1 Hz, 1H), 7.84 (d, J=2.1 Hz, 1H), 4.26 (q, J₁=14.1 Hz, J₂=7.2 Hz, 2H), 3.87 (t, J=5.4 Hz, 4H), 3.45 (t, J=5.4 Hz, 4H), 1.35 (t, J=6.6 Hz, 3H)

MS (ESI⁺) m/z 351 (M+55)

### Example 23.2

### Preparation of ethyl 2-amino-5-morpholinopyridin-3-yl carbamate

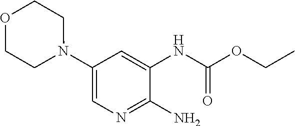 [SMILES: CCOC(=O)Nc1cc(N2CCOCC2)cnc1N]

The mixture of the product from example 23.1 (900 mg, 3 mmol) and Pd/C (90 mg) in EtOH (10 ml) was stirred at room temperature under H₂ atmosphere for 18 hour. The mixture was filtered and the filtrate was concentrated in vacuo to give titled compound (730 mg, 90%).

¹H NMR (300 M Hz, CDCl₃) δ 7.57 (s, 2H), 6.61 (s, 1H), 4.24 (q, J₁=14.1 Hz, J₂=6.9 Hz, 2H), 3.84 (t, J=4.8 Hz, 4H), 3.05 (t, J=4.8 Hz, 4H), 1.32 (t, J=6.9 Hz, 3H)

### Example 23.3

### Preparation of methyl 9-(ethoxycarbonylamino)-3-hydroxy-7-morpholino-4-oxo-4,9a-dihydro-3H-pyrido[1,2-a]pyrimidine-2-carboxylate

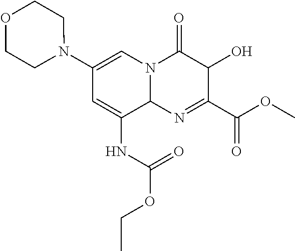 [SMILES: CCOC(=O)NC1=CC(N2CCOCC2)=CN2C(=O)C(O)C(C(=O)OC)=NC12]

The mixture of the product from example 23.2 (120 mg, 0.45 mmol), DAF (176 mg, 0.67 mmol) and AcOH (13 mg, 0.23 mmol) in MeOH (0.5 ml) was heated at 80° C. for 18 hours. After cooling down to room temperature, the solids were collected by filtration, washed with MeOH and dried in vacuo to give the desired product (20 mg, 11.5%).

¹H NMR (300 M Hz, DMSO-d⁶) δ 8.64 (s, 1H), 8.12 (d, J=2.4 Hz, 1H), 7.72 (d, J=2.4 Hz, 1H), 4.21 (q, J₁=14.1 Hz, J₂=6.9 Hz, 2H), 3.90 (s, 3H), 3.84-3.72 (m, 4H), 3.17-3.06 (m, 4H), 1.27 (t, J=6.9 Hz, 3H)

MS (ESI⁻) m/z 393 (M−1)

### Example 23.4

### Preparation of ethyl 2-(4-fluorobenzylcarbamoyl)-3-hydroxy-7-morpholino-4-oxo-4,9a-dihydro-3H-pyrido[1,2-a]pyrimidin-9-yl carbamate

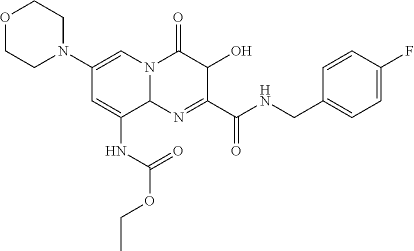 [SMILES: CCOC(=O)NC1=CC(N2CCOCC2)=CN2C(=O)C(O)C(C(=O)NCc3ccc(F)cc3)=NC12]

The solution of the product from example 23.3 (100 mg, 0.25 mmol) and (4-fluorophenyl)methan amine (158 mg, 1.27 mmol) in MeOH (2 ml) was heated at reflux for 3 h. After cooling down to room temperature, MeOH was concentrated in vacuo. The solids were collected by filtration, washed with MeOH and dried in vacuo to give the desired product (30 mg, 25%).

¹H NMR (300 M Hz, DMSO-d⁶) δ 12.41 (s, 1H), 10.41 (t, J=6.3 Hz, 1H), 9.90 (s, 1H), 8.32 (d, J=2.1 Hz, 1H), 7.76 (d, J=2.1 Hz, 1H), 7.38 (dd, J₁=8.7 Hz, J₂=5.7 Hz, 2H), 7.19 (t, J=8.7 Hz, 2H), 4.60 (d, J=6.0 Hz, 2H), 4.25 (m, 2H), 3.84-3.72 (m, 4H), 3.17-3.06 (m, 4H), 1.29 (t, J=7.2 Hz, 3H)

MS (ESI⁻) m/z 486 (M−1)

### Example 24

### Preparation of N-(4-fluorobenzyl)-9-(1,3-propanesultam)-3-hydroxyl-7-morpholino-4-oxo-4H-pyrido[1,2-a]pyrimidine-2-carboxamide

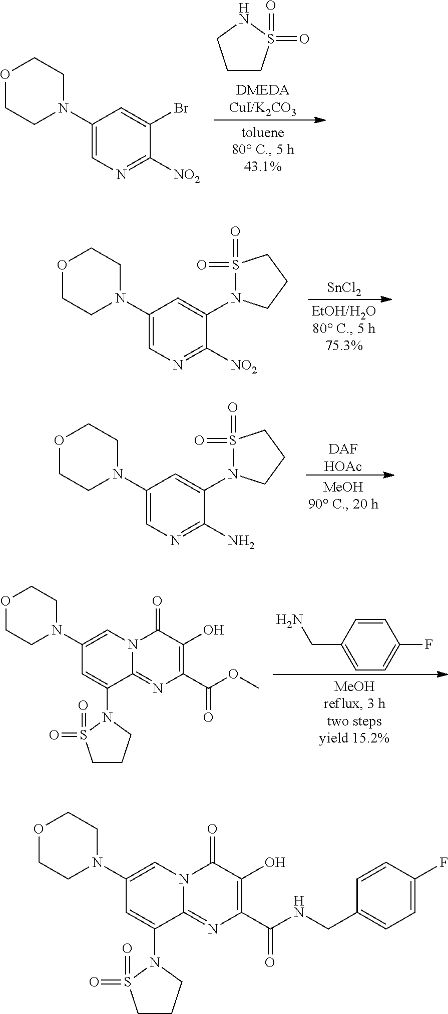

4-(5-Bromo-6-nitropyridin-3-yl)morpholine was prepared as described in example 21.1 Isothiazolidine 1,1-dioxide was made according to the literature: J. Org. Chem., 52, 11, 1987, 2162-2166.

### Example 24.1

### Preparation of 4-[5-(1,3-propanesultam)-6-nitropyridin-3-yl]morpholine

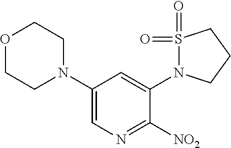 [SMILES: O=[N+]([O-])c1ncc(N2CCOCC2)cc1N1CCCS1(=O)=O]

To the mixture of 4-(5-bromo-6-nitropyridin-3-yl)morpholine (1.00 g, 3.47 mmol) in toluene (20 ml), isothiazolidine 1,1-dioxide (0.63 g, 5.21 mmol), CuI (0.17 g, 0.87 mmol), DMEDA (0.15 g, 1.74 mmol) and K₂CO₃ (0.96 g, 6.94 mmol) were added successively. The mixture was heated at 80° C. for 5 h under nitrogen atmosphere. After cooling down to room temperature, the mixture was filtrated and the filtrate was concentrated under reduced pressure. The residue was purified by column chromatography (DCM/MeOH=100/1) to give the desired product (0.49 g, 43.1%).

¹H NMR (300 MHz, d-DMSO) δ 8.16 (d, J=2.7 Hz, 1H), 7.51 (d, J=2.7 Hz, 1H), 3.79-3.72 (m, 6H), 3.43-3.34 (m, 6H), 2.43 (m, 2H).

MS (ESI⁺) m/z 329 (M+1)

### Example 24.2

### Preparation of 3-(1,3-propanesultam)-5-morpholinopyridin-2-amine

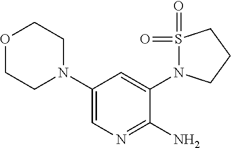 [SMILES: Nc1ncc(N2CCOCC2)cc1N1CCCS1(=O)=O]

To the solution of the product from example 24.1 (1.20 g, 3.66 mmol) in EtOH (20 ml), SnCl₂.2H₂O (2.48 g, 10.98 mmol) was added. The mixture was heated at 80° C. for 5 h. After cooling down to room temperature, the mixture was adjusted pH to 9-10 with KOH (20%) and then filtrated. The filtrate was concentrated under reduced pressure. The residue was purified by column chromatography (DCM/MeOH=50/1) to afford the product as a brown solid (0.82 g, 75.3%).

¹H NMR (300 MHz, d-DMSO) δ 7.67 (d, J=3.0 Hz, 1H), 7.23 (d, J=3.0 Hz, 1H), 5.52 (s, 2H), 3.71-3.67 (m, 4H), 3.53 (t, J=6.9 Hz, 2H), 3.40 (t, J=7.5 Hz, 2H), 2.94-2.89 (m, 4H), 2.41-2.35 (m, 2H).

MS (ESI⁺) m/z 299 (M+1)

### Example 24.3

### Preparation of methyl 9-(1,3-propanesultam)-3-hydroxy-7-morpholino-4-oxo-4H-pyrido[1,2-a]pyrimidine-2-carboxylate

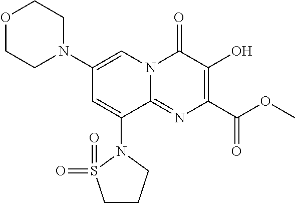 [SMILES: COC(=O)c1nc2c(N3CCCS3(=O)=O)cc(N3CCOCC3)cn2c(=O)c1O]

The product from example 24.2 (0.55 g, 1.85 mmol), DAF (0.96 g, 3.69 mmol) and HOAc (0.02 g 0.37 mmol) were mixed in MeOH (1.2 ml). The mixture was heated at 90° C. for 20 h. This mixture was used directly in the next step.

### Example 24.4

### Preparation of N-(4-fluorobenzyl)-9-(1,3-propanesultam)-3-hydroxy-7-morpholino-4-oxo-4H-pyrido[1,2-a]pyrimidine-2-carboxamide

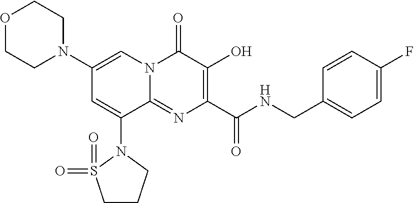 [SMILES: O=C(NCc1ccc(F)cc1)c1nc2c(N3CCCS3(=O)=O)cc(N3CCOCC3)cn2c(=O)c1O]

The mixture from example 24.3 was diluted with MeOH (20 ml) and then (4-fluorophenyl)methan-amine (0.23 g, 1.84 mmol) was added. The mixture was heated at reflux for 3 h. After cooling down to room temperature, the resulting precipitates were collected by filtration, washed with MeOH and dried in vacuo to afford the product as a yellow solid (0.14 g, two steps yield 15.2%).

¹H NMR (300 MHz, d-DMSO) δ 11.89 (s, 1H), 9.70 (t, J=6.3 Hz, 1H), 7.82 (d, J=1.8 Hz, 1H), 7.53 (d, J=1.8 Hz, 1H), 7.35 (m, 2H), 7.16 (t, J=8.7 Hz, 2H), 4.53 (d, J=6.6 Hz, 2H), 3.98 (t, J=6.3 Hz, 2H), 3.77-3.74 (m, 4H), 3.47 (t, J=6.9 Hz, 2H), 3.21-3.18 (m, 4H), 2.47-2.44 (m, 2H).

MS (ESI⁻) m/z 516 (M−1)

### Example 25

### Preparation of N-(3,4-dichlorobenzyl)-9-(1,3-propanesultam)-3-hydroxy-7-morpholino-4-oxo-4H-pyrido[1,2-a]pyrimidine-2-carboxamide

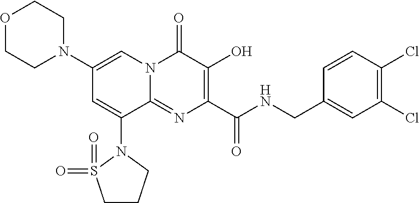 [SMILES: O=C(NCc1ccc(Cl)c(Cl)c1)c1nc2c(N3CCCS3(=O)=O)cc(N3CCOCC3)cn2c(=O)c1O]

This target was made in a similar way to Example 24.

¹H NMR (300 MHz, d-DMSO) δ 11.74 (s, 1H), 9.71 (t, J=6.3 Hz, 1H), 7.82 (d, J=1.8 Hz, 1H), 7.62-7.57 (m, 2H), 7.53 (d, J=1.8 Hz, 1H), 7.33-7.28 (m, 1H), 4.57 (d, J=6.0 Hz, 2H), 3.99 (t, J=6.3 Hz, 2H), 3.78-3.74 (m, 4H), 3.48 (t, J=6.9 Hz, 2H), 3.20-3.14 (m, 4H), 2.47-2.42 (m, 2H).

MS (ESI⁻) m/z 566 (M−1)

### Example 26

### Preparation of preparation of 9-acetamido-N-(4-fluorobenzyl)-3-hydroxy-7-morpholino-4-oxo-4H-pyrido[1,2-a]pyrimidine-2-carboxamide

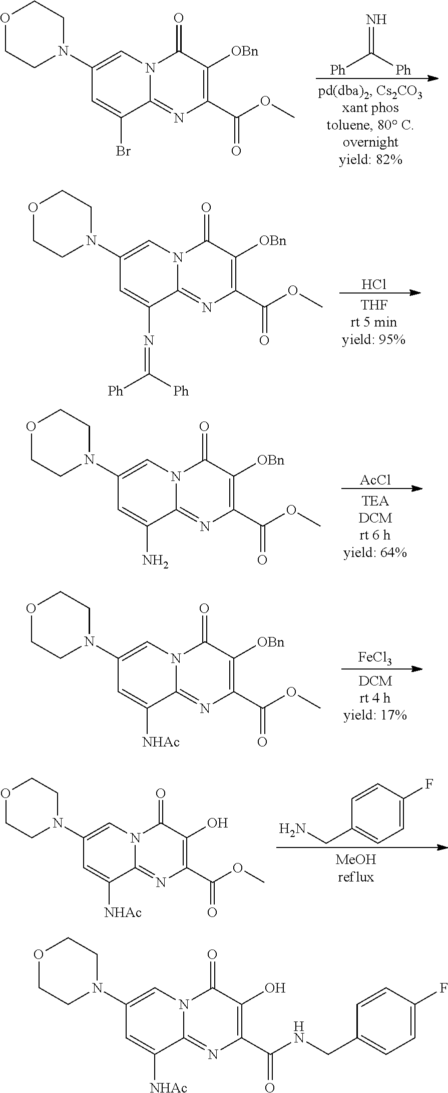

### Example 26.1

### Preparation of 9-acetamido-N-(4-fluorobenzyl)-3-hydroxy-7-morpholino-4-oxo-4H-pyrido[1,2-a]pyrimidine-2-carboxamide

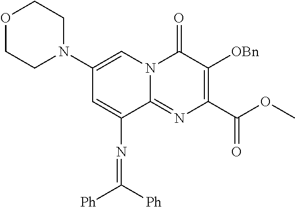 [SMILES: COC(=O)c1nc2c(N=C(c3ccccc3)c3ccccc3)cc(N3CCOCC3)cn2c(=O)c1OCc1ccccc1]

Methyl-3-(benzyloxy)-9-bromo-7-morpholino-4-oxo-4H-pyrido[1,2-a]pyrimidine-2-carboxylate (471 mg, 1.0 mmol), diphenylmethanimine (0.2 ml, 1.2 mmol), Pd₂ dba₃ (46 mg, 0.05 mmol), xantphos (58 mg, 0.1 mmol) and Cs₂CO₃ (489 mg, 1.5 mmol) were mixed in toluene (10 ml). The mixture was heated under N₂ atmosphere at 80° C. for 18 h. After cooling down to room temperature, toluene was removed in vacuo. The residue was purified by column chromatography (DCM/MeOH=50/1) to give the desired product (470 mg, 82%).

¹H NMR (300 M Hz, CDC1) δ 7.91 (d, J=2.4 Hz, 1H), 7.75-7.65 (m, 2H), 7.63-7.20 (m, 3H), 7.41-7.31 (m, 6H), 7.31-7.20 (m, 3H), 7.19-7.09 (m, 2H), 5.12 (s, 2H), 3.76 (s, 3H), 3.73-3.62 (m, 4H), 3.16-3.02 (m, 4H)

MS (ESI⁺) m/z 597 (M+23)

### Example 26.2

### Preparation of methyl 9-amino-3-(benzyloxy)-7-morpholino-4-oxo-4H-pyrido[1,2-a]pyrimidine-2-carboxylate

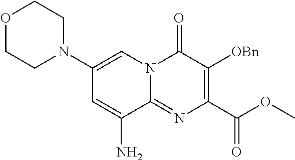 [SMILES: COC(=O)c1nc2c(N)cc(N3CCOCC3)cn2c(=O)c1OCc1ccccc1]

To the solution of the product from 26.1 (100 mg, 0.17 mmol) in THF (2 ml), 1N HCl (0.068 ml) was added. After stirring at room temperature for 5 minutes, aqueous NaHCO₃ solution was added. The mixture was extracted with DCM three times. The organic phase were combined, washed with water, dried and concentrated in vacuo. The residue was purified by gel column chromatography (PE/EA=1/1) to give the title compound (68 mg, 95%).

¹H NMR (300 M Hz, DMSO-d⁶) δ 7.71 (d, J=2.4 Hz, 1H), 7.42-7.30 (m, 5H), 6.91 (d, J=2.4 Hz, 1H), 6.11 (s, 2H), 5.13 (s, 2H), 3.79 (s, 3H), 3.76 (t, J=4.5 Hz, 4H), 3.13 (t, J=4.5 Hz, 4H)

### Example 26.3

### Preparation of methyl 9-acetamido-3-(benzyloxy)-7-morpholino-4-oxo-4H-pyrido[1,2-a]pyrimidine-2-carboxylate

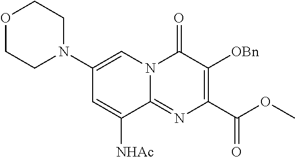 [SMILES: COC(=O)c1nc2c(NC(C)=O)cc(N3CCOCC3)cn2c(=O)c1OCc1ccccc1]

To the solution of the product from example 26.2 (350 mg, 0.9 mmol) in DCM (10 ml), TEA (370 mg, 3.66 mmol) and AcCl (143 mg, 1.83 mmol) were added successively at 0° C. After stirring at room temperature for 6 hours, water was added. The organic phase was washed with saturated brine three times, dried over Na₂SO₄ and concentrated in vacuo. The residue was purified by column chromatography (EA/PE=1/1) to give the desired product (250 mg, 64%).

¹H NMR (300 M Hz, CDC1) δ 9.26 (s, 1H), 8.81 (d, J=2.1 Hz, 1H), 8.04 (d, J=2.1 Hz, 1H), 7.51-7.45 (m, 2H), 7.39-7.33 (m, 3H), 5.30 (s, 2H), 4.02-3.83 (m, 7H), 3.34-3.19 (m, 4H), 2.31 (s, 3H)

### Example 26.4

### Preparation of methyl 9-acetamido-3-hydroxy-7-morpholino-4-oxo-4H-pyrido[1,2-a]pyrimidine-2-carboxylate

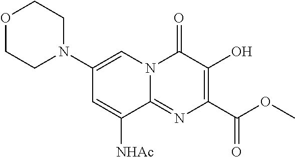 [SMILES: COC(=O)c1nc2c(NC(C)=O)cc(N3CCOCC3)cn2c(=O)c1O]

The product from example 26.3 (300 mg, 0.66 mmol) and FeCl₃ (535 mg, 3.3 mmol) were mixed in DCM (5 ml). After stirring at room temperature for 4 hours, dilute HCl was added. The organic phase was washed with saturated brine three times, dried over Na₂SO₄ and concentrated in vacuo. The residue was washed with EA to give the desired product (50 mg, 17%).

¹H NMR (300 M Hz, DMSO-d⁶) δ 10.24 (s, 1H), 9.66-9.58 (m, 1H), 8.49 (d, J=1.5 Hz, 1H), 7.78 (d, J=1.5 Hz, 1H), 3.88 (s, 3H), 3.82-3.71 (m, 4H), 3.19-3.06 (m, 4H), 2.25 (s, 3H)

### Example 26.5

### Preparation of 9-acetamido-N-(4-fluorobenzyl)-3-hydroxy-7-morpholino-4-oxo-4H-pyrido[1,2-a]pyrimidine-2-carboxamide

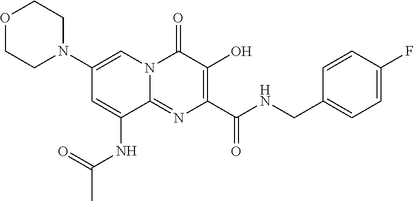 [SMILES: CC(=O)Nc1cc(N2CCOCC2)cn2c(=O)c(O)c(C(=O)NCc3ccc(F)cc3)nc12]

The solution of the product from example 26.4 (40 mg, 0.11 mmol) and (4-fluorophenyl)methan amine (69 mg, 0.55 mmol) in MeOH (3 ml) was heated at reflux for 3 h. After cooling down to room temperature, MeOH was concentrated in vacuo. The solids were collected by filtration, washed with MeOH and dried in vacuo to give the desired product (11 mg, 22%).

¹H NMR (300 M Hz, CDCl) δ 12.19-11.09 (m, 1H), 8.91-8.76 (m, 1H), 8.67 (d, J=1.8 Hz, 1H), 8.02 (s, 1H), 7.93 (d, J=1.8 Hz, 1H), 7.41-7.29 (m, 2H), 7.03 (t, J=8.1 Hz, 2H), 4.67 (s, 2H), 3.96-3.75 (m, 4H), 3.29-3.06 (m, 4H), 2.27 (s, 3H)

MS (ESI⁺) m/z 478 (M+23)

### Example 27

### Preparation of N-(4-fluorobenzyl)-9-(2-(dimethylamino)-2-oxoacetamido)-3-hydroxy-7-morpholino-4-oxo-4H-pyrido[1,2-a]pyrimidine-2-carboxamide

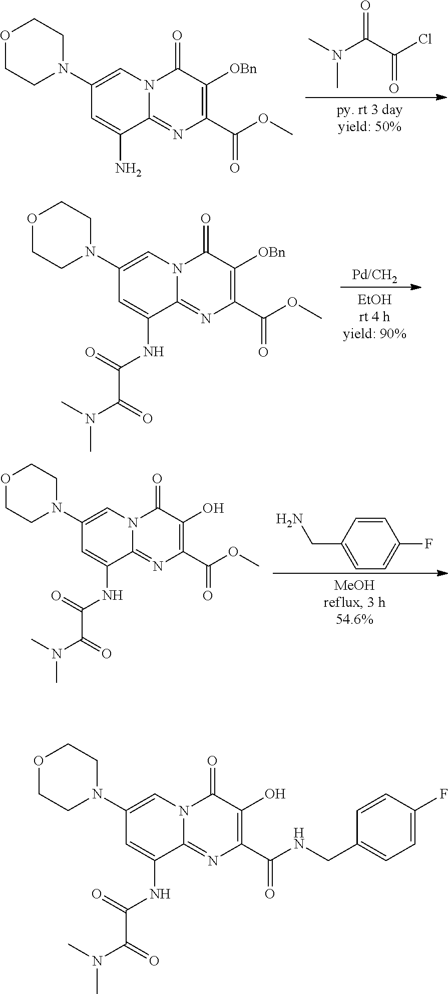

### Example 27.1

### Preparation of methyl 3-(benzyloxy)-9-(2-(dimethylamino)-2-oxo-acetamido)-7-morpholino-4-oxo-4H-pyrido[1,2-a]pyrimidine-2-carboxylate

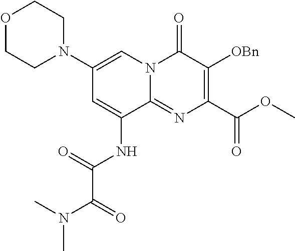 [SMILES: COC(=O)c1nc2c(NC(=O)C(=O)N(C)C)cc(N3CCOCC3)cn2c(=O)c1OCc1ccccc1]

To the solution of the product from example 26.2 (0.12 g, 0.29 mmol) and TEA (0.11 g, 0.88 mmol) in DCM (5 ml), 2-(dimethylamino)-2-oxoacetyl chloride (49 mg, 0.35 mmol) was added. The mixture was heated at 40° C. for 3 h. After cooling down to room temperature, the mixture was concentrated under reduced pressure and EA (20 ml) was added. The mixture was washed with H₂O (3×20 ml) and brine successively, dried over anhydrous sodium sulfate and concentrated in vacuo. The residue was purified by column chromatography (PE/EA=4/1) to give the crude product (0.11 g, crude yield 73.3%).

MS (ESI⁺) m/z 510 (M+1)

### Example 27.2

### Preparation of methyl 9-(2-(dimethylamino)-2-oxoacetamido)-3-hydroxy-7-morpholino-4-oxo-4H-pyrido[1,2-a]pyrimidine-2-carboxylate

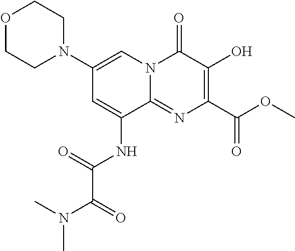 [SMILES: COC(=O)c1nc2c(NC(=O)C(=O)N(C)C)cc(N3CCOCC3)cn2c(=O)c1O]

This compound was prepared by adapting the procedure described in Example 26.4.

¹H NMR (300 MHz, d-DMSO) δ 10.66 (s, 1H), 10.33 (s, 1H), 8.47 (d, J=2.7 Hz, 1H), 7.80 (d, J=2.4 Hz, 1H), 3.88 (s, 3H), 3.80-3.75 (m, 4H), 3.26 (s, 3H), 3.17-3.13 (m, 4H), 2.97 (s, 3H).

MS (ESI⁺) m/z 418 (M−1)

### Example 27.3

### Preparation of N-(4-fluorobenzyl)-9-(2-(dimethylamino)-2-oxoacetamido)-3-hydroxy-7-morpholino-4-oxo-4H-pyrido[1,2-a]pyrimidine-2-carboxamide

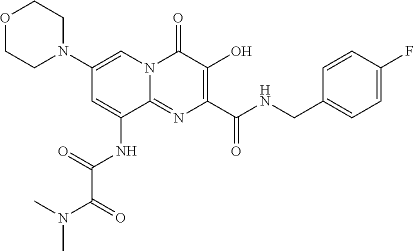 [SMILES: CN(C)C(=O)C(=O)Nc1cc(N2CCOCC2)cn2c(=O)c(O)c(C(=O)NCc3ccc(F)cc3)nc12]

This product was made by adapting the procedure described in Example 26.5.

¹H NMR (300 MHz, d-DMSO) δ 12.23 (s, 1H), 10.82 (s, 1H), 10.08 (t, J=6.3 Hz, 1H), 8.45 (d, J=1.8 Hz 1H), 7.82 (d, J=2.4 Hz, 1H), 7.42-7.37 (m, 2H), 7.23-7.16 (m, 3H), 4.60 (d, J=6.3 Hz, 2H), 3.80-3.74 (m, 4H), 3.15-3.12 (m, 4H), 2.92 (s, 3H), 2.91 (s, 3H).

MS (ESI⁻) m/z 511 (M−1)

### Example 28

### Preparation of N-(4-fluorobenzyl)-3-hydroxy-9-(N-methyl acetamido)-7-morpholino-4-oxo-4H-pyrido[1,2-a]pyrimidine-2-carboxamide

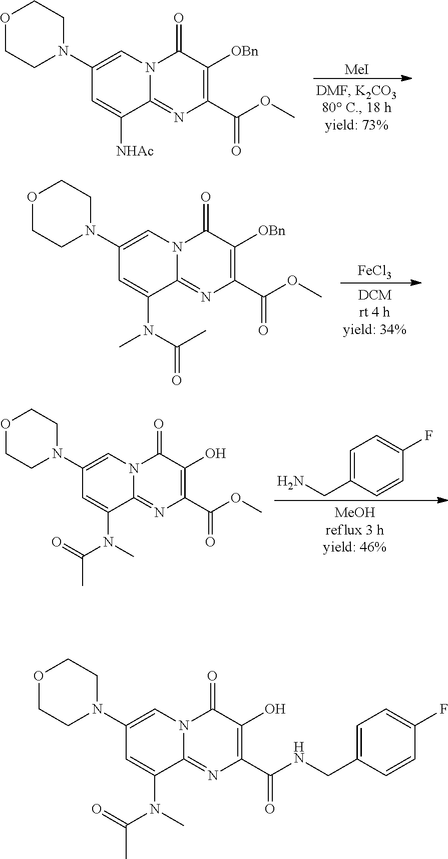

### Example 28.1

### Preparation of methyl 3-(benzyloxy)-9-(N-methylacetamido)-7-morpholino-4-oxo-4H-pyrido[1,2-a]pyrimidine-2-carboxylate

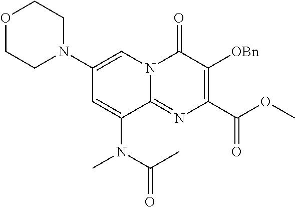 [SMILES: COC(=O)c1nc2c(N(C)C(C)=O)cc(N3CCOCC3)cn2c(=O)c1OCc1ccccc1]

The product from example 26.3 (500 mg, 0.1 mmol), MeI (669 mg, 4.71 mmol), and K₂CO₃ (976 mg, 7.07 mmol) were mixed in DMF (10 ml). After stirring at 80° C. for 18 hours, water was added. The reaction mixture was extracted with EA three times. The organic layers were combined, dried and concentrated in vacuo. The residue was purified by gel column chromatography (DCM/MeOH=30/1) to give the title compound (380 mg, 68%).

¹H NMR (300 M Hz, CDCl) δ 8.36 (d, J=1.8 Hz, 1H), 7.57 (d, J=1.8 Hz, 1H), 7.55-7.47 (m, 2H), 7.43-7.29 (m, 3H), 5.32 (s, 2H), 3.95-3.84 (m, 7H), 3.32 (s, 3H), 3.29-3.18 (m, 4H), 1.97 (s, 3H)

MS (ESI⁺) m/z 489 (M+23)

### Example 28.2

### Preparation of methyl 3-hydroxy-9-(N-methylacetamido)-7-morpholino-4-oxo-4H-pyrido[1,2-a]pyrimidine-2-carboxylate

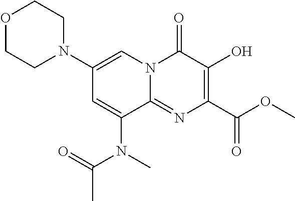 [SMILES: COC(=O)c1nc2c(N(C)C(C)=O)cc(N3CCOCC3)cn2c(=O)c1O]

The crude product was made by adapting the procedure described in Example 26.4, which was used for the next step directly.

### Example 28.3

### Preparation of N-(4-fluorobenzyl)-3-hydroxy-9-(N-methyl acetamido)-7-morpholino-4-oxo-4H-pyrido[1,2-a]pyrimidine-2-carboxamide

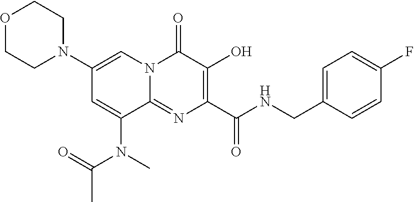 [SMILES: CC(=O)N(C)c1cc(N2CCOCC2)cn2c(=O)c(O)c(C(=O)NCc3ccc(F)cc3)nc12]

This target was made by adapting the procedure described in the Example 26.5.

¹H NMR (300 M Hz, DMSO-d⁶) δ 12.11 (s, 1H), 8.99 (t, J=1.8 Hz, 1H), 8.10-7.99 (m, 2H), 7.43-7.36 (m, 2H), 7.17 (t, J=8.7 Hz, 2H), 4.56 (d, J=6.0 Hz, 2H), 3.81-3.72 (m, 4H), 3.25-3.04 (m, 7H), 1.81 (s, 3H)

### Example 29

### Preparation of N-(4-fluorobenzyl)-3-hydroxy-9-(3-methyl-2-oxoimidazolidin-1-yl)-7-morpholino-4-oxo-4H-pyrido[1,2-a]pyrimidine-2-carboxamide

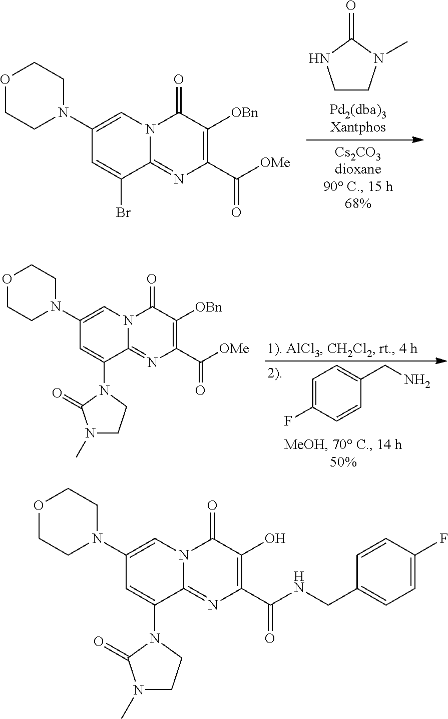

### Example 29.1

### Preparation of methyl 3-(benzyloxy)-9-(3-methyl-2-oxoimidazolidin-1-yl)-7-morpholino-4-oxo-4H-pyrido[1,2-a]pyrimidine-2-carboxylate

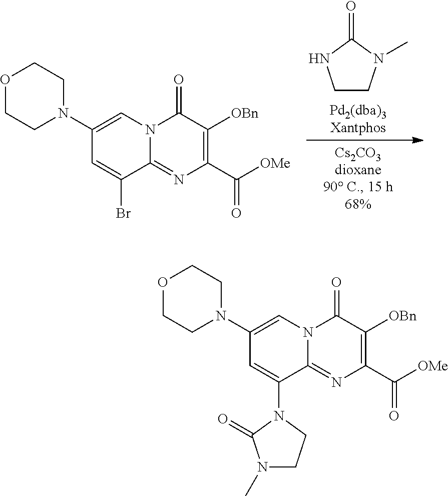

This compound was prepared by adapting the procedure described in Example 2.1

¹H NMR (300 MHz, CDCl₃) δ 8.24 (d, J=1.8 Hz, 1H), 7.95 (d, J=1.8 Hz, 1H), 7.53-7.48 (m, 2H), 7.41-7.30 (m, 3H), 5.31 (s, 2H), 4.33 (t, J=8.1 Hz, 2H), 3.91-3.84 (m, 7H), 3.54 (t, J=8.1 Hz, 3H), 3.24 (t, J=4.2 Hz, 4H), 2.93 (s, 3H).

MS (ESI⁺) m/z 494 (M+1), 516 (M+23)

### Example 29.2

### Preparation of N-(4-fluorobenzyl)-3-hydroxy-9-(3-methyl-2-oxoimidazolidin-1-yl)-7-morpholino-4-oxo-4H-pyrido[1,2-a]pyrimidine-2-carboxamide

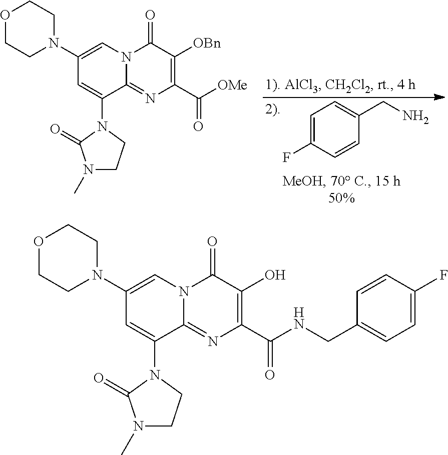

The product from Example 29.1 (100 mg, 0.20 mmol) and aluminum trichloride (162 mg, 1.22 mmol) were mixed in dichloromethane (5 ml). The mixture was stirred at room temperature for 4 h. Then water (10 ml) was added and the mixture was extracted with dichloromethane. The organic layers were washed with 1N HCl three times, dried over anhydrous sodium sulfate and evaporated under reduced pressure. The residue and (4-fluorophenyl)methanamine (33 mg, 0.26 mmol) were mixed in methanol (5 ml) and the mixture was heated at 70° C. for 15 h. After cooling down to room temperature, 6N HCl was added dropwise till the pH of the mixture was about 4. The solids were collected by filtration, washed with methanol and dried in vacuo to afford the desired compound (50 mg, 50%).

¹H NMR (300 MHz, DMSO-d⁶) δ 12.01 (s, 1H), 9.14 (t, J=6.0 Hz, 1H), 7.95 (d, J=2.1 Hz, 1H), 7.78 (d, J=2.1 Hz, 1H), 7.45-7.36 (m, 2H), 7.19 (t, J=8.7 Hz, 2H), 4.55 (d, J=6.0 Hz, 2H), 3.99 (t, J=8.7 Hz, 2H), 3.76 (t, J=4.5 Hz, 4H), 3.49 (t, J=8.7 Hz, 2H), 3.15 (t, J=4.5 Hz, 4H), 2.77 (s, 3H).

MS (ESI⁺) m/z 497 (M+1), 519 (M+23), 551 (M+55)

### Example 30

### Preparation of N-(3,4-dichlorobenzyl)-3-hydroxy-9-(3-methyl-2-oxoimidazolidin-1-yl)-7-morpholino-4-oxo-4H-pyrido[1,2-a]pyrimidine-2-carboxamide

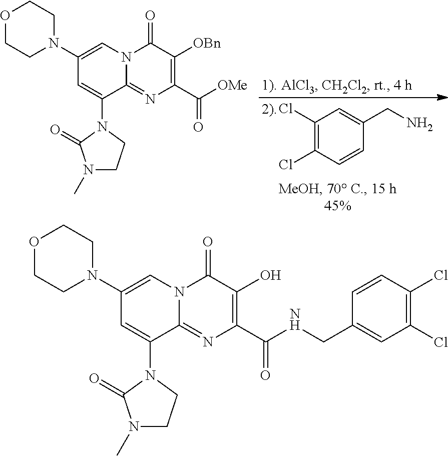

This compound was prepared by adapting the procedure described in Example 29.2

¹H NMR (300 MHz, DMSO-d⁶) δ 11.87 (s, 1H), 9.23 (t, J=6.0 Hz, 1H), 7.95 (d, J=2.1 Hz, 1H), 7.78 (d, J=2.1 Hz, 1H), 7.66-7.60 (m, 2H), 7.38-7.33 (m, 1H), 4.57 (d, J=6.0 Hz, 2H), 4.03 (t, J=8.1 Hz, 2H), 3.76 (t, J=4.2 Hz, 4H), 3.53 (t, J=8.1 Hz, 2H), 3.15 (t, J=4.2 Hz, 4H), 2.79 (s, 3H).

MS (ESI⁺) m/z 547 (M+1), 569 (M+23)

### Example 31

### Preparation of 7-Dimethylamino-3-hydroxy-9-(3-methyl-2-oxo-imidazolidin-1-yl)-4-oxo-4H-pyrido[1,2-a]pyrimidine-2-carboxylic acid 4-fluoro-benzylamide

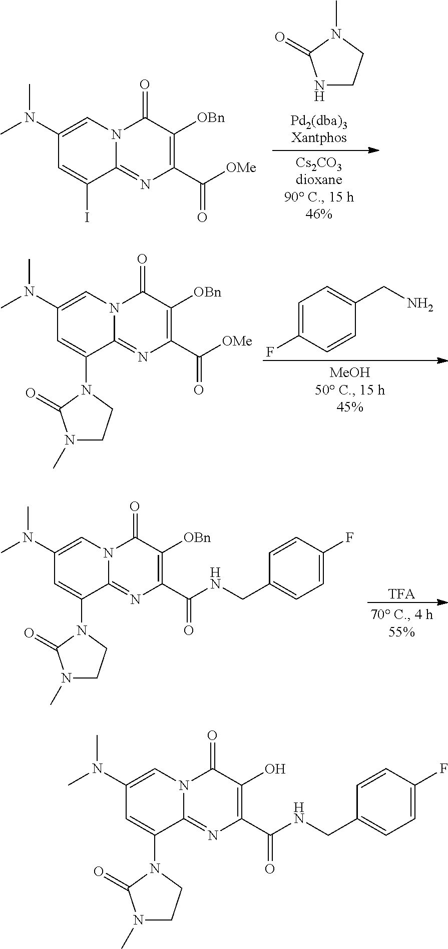

This target was made by adapting the procedure described in Example 7.

### Example 31.1

### Preparation of 3-Benzyloxy-7-dimethylamino-9-(3-methyl-2-oxo-imidazolidin-1-yl)-4-oxo-4H-pyrido[1,2-a]pyrimidine-2-carboxylic acid methyl ester

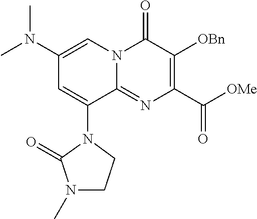 [SMILES: COC(=O)c1nc2c(N3CCN(C)C3=O)cc(N(C)C)cn2c(=O)c1OCc1ccccc1]

¹H NMR (300 MHz, DMSO-d⁶) δ 7.99 (d, J=2.2 Hz, 1H), 7.89 (d, J=2.2 Hz, 1H), 7.50-7.30 (m, 5H), 4.07 (t, J=7.5 Hz, 2H), 3.82 (s, 3H), 3.47 (t, J=8.1 Hz, 2H), 3.02 (s, 6H), 2.80 (s, 3H).

MS (ESI⁺) m/z 452 (M+1)

### Example 31.2

### Preparation of 3-Benzyloxy-7-dimethylamino-9-(3-methyl-2-oxo-imidazolidin-1-yl)-4-oxo-4H-pyrido[1,2-a]pyrimidine-2-carboxylic acid 4-fluoro-benzylamide

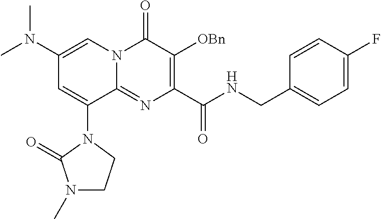 [SMILES: CN1CCN(c2cc(N(C)C)cn3c(=O)c(OCc4ccccc4)c(C(=O)NCc4ccc(F)cc4)nc23)C1=O]

The crude product was used in the next step.

MS (ESI⁺) m/z 545 (M+1)

### Example 31.3

### Preparation of 7-Dimethylamino-3-hydroxy-9-(3-methyl-2-oxo-imidazolidin-1-yl)-4-oxo-4H-pyrido[1,2-a]pyrimidine-2-carboxylic acid 4-fluoro-benzylamide

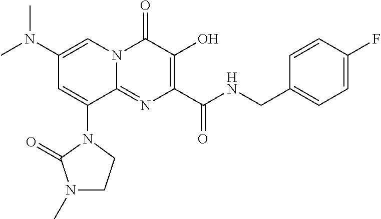 [SMILES: CN1CCN(c2cc(N(C)C)cn3c(=O)c(O)c(C(=O)NCc4ccc(F)cc4)nc23)C1=O]

¹H NMR (300 MHz, DMSO-d⁶) δ 11.89 (s, 1H), 9.07 (t, J=6.2 Hz, 1H), 7.78 (d, J=2.6 Hz, 1H), 7.72 (d, J=2.6 Hz, 1H), 7.41 (dd, J=5.9, 8.2 Hz, 2H), 7.19 (t, J=8.6 Hz, 2H), 4.56 (d, J=6.2 Hz, 2H), 4.01 (t, J=7.0 Hz, 2H), 3.49 (t, J=7.9 Hz, 2H), 2.96 (s, 6H), 2.77 (s, 3H).

MS (ESI⁺) m/z 455 (M+1)

### Example 32

### Preparation of 3-Hydroxy-7-(4-isopropyl-piperazin-1-yl)-9-(3-methyl-2-oxo-imidazolidin-1-yl)-4-oxo-4H-pyrido[1,2-a]pyrimidine-2-carboxylic acid 4-fluoro-benzylamide

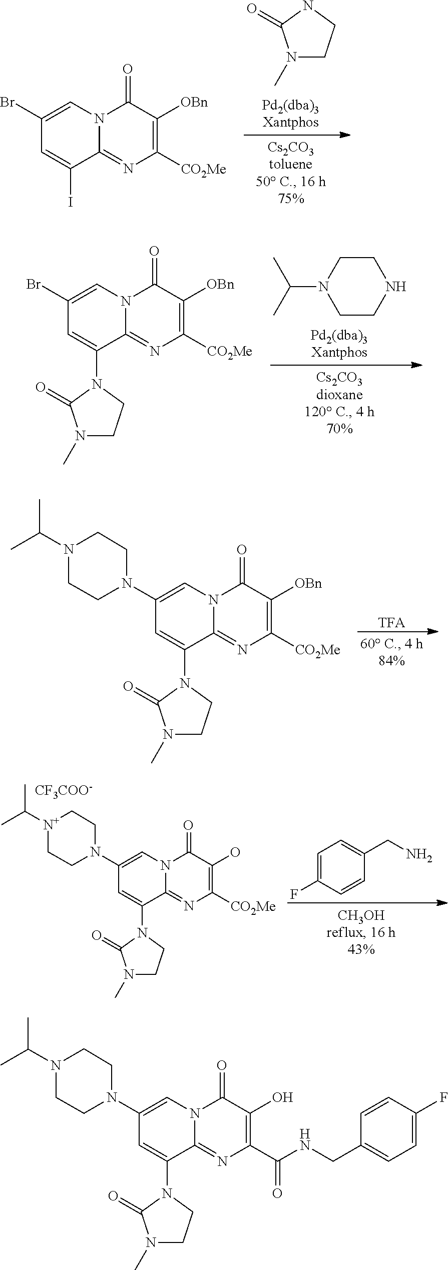

This target was made by adapting the procedure described in Example 2.

### Example 32.1

### Preparation of 3-Benzyloxy-7-bromo-9-(3-methyl-2-oxo-imidazolidin-1-yl)-4-oxo-4H-pyrido[1,2-a]pyrimidine-2-carboxylic acid methyl ester

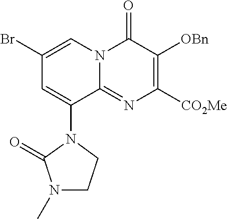 [SMILES: COC(=O)c1nc2c(N3CCN(C)C3=O)cc(Br)cn2c(=O)c1OCc1ccccc1]

This compound was prepared by adapting the procedure described in example 2.1.

¹H NMR (300 MHz, CDCl₃) δ 8.96 (s, 1H), 8.09 (s, 1H), 7.52-7.43 (m, 2H), 7.41-7.31 (m, 3H), 5.34 (s, 2H), 4.31 (t, J=8.0 Hz, 2H), 3.89 (s, 3H), 3.54 (t, J=8.0 Hz, 2H), 2.93 (s, 3H).

MS (ESI⁺) m/z 487 (M[Br⁷⁹]+1), 489 (M[Br⁸¹]+1)

### Example 32.2

### Preparation of 3-Benzyloxy-7-(4-isopropyl-piperazin-1-yl)-9-(3-methyl-2-oxo-imidazolidin-1-yl)-4-oxo-4H-pyrido[1,2-a]pyrimidine-2-carboxylic acid methyl ester

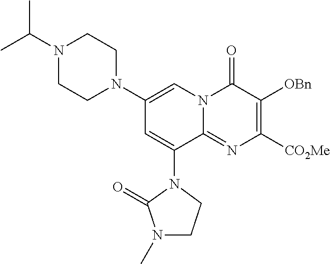 [SMILES: COC(=O)c1nc2c(N3CCN(C)C3=O)cc(N3CCN(C(C)C)CC3)cn2c(=O)c1OCc1ccccc1]

This compound was prepared by adapting the procedure described in example 2.2.

¹H NMR (300 MHz, CDCl₃) δ 8.23 (s, 1H), 7.95 (s, 1H), 7.52-7.43 (m, 2H), 7.41-7.31 (m, 3H), 5.30 (s, 2H), 4.31 (t, J=8.0 Hz, 2H), 3.89 (s, 3H), 3.54 (t, J=7.7 Hz, 2H), 3.29 (s, 4H), 2.93 (s, 3H), 2.68 (s, 5H), 1.09 (s, 6H).

MS (ESI⁺) m/z 535 (M+1)

### Example 32.3

### Preparation of 3-Hydroxy-7-(4-isopropyl-piperazin-1-yl)-9-(3-methyl-2-oxo-imidazolidin-1-yl)-4-oxo-4H-pyrido[1,2-a]pyrimidine-2-carboxylic acid methyl ester

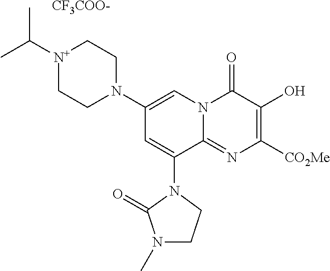

This compound was prepared by adapting the procedure described in example 2.3.

¹H NMR (300 MHz, DMSO-d⁶) δ 10.26 (s, 1H), 9.50 (s, 1H), 7.98 (d, J=2.5 Hz, 1H), 7.81 (d, J=2.5 Hz, 1H), 4.18-4.08 (m, 2H), 3.94-3.85 (m, 5H), 3.64-3.45 (m, 5H), 3.29-3.16 (m, 2H), 3.11-2.98 (m, 2H), 2.81 (s, 3H), 1.30 (d, J=6.6 Hz, 6H).

MS (ESI⁺) m/z 445 (M-TFA+1)

### Example 32.4

### Preparation of 3-Hydroxy-7-(4-isopropyl-piperazin-1-yl)-9-(3-methyl-2-oxo-imidazolidin-1-yl)-4-oxo-4H-pyrido[1,2-a]pyrimidine-2-carboxylic acid 4-fluoro-benzylamide

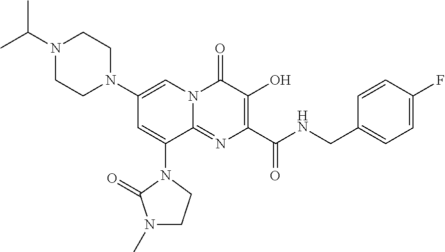 [SMILES: CC(C)N1CCN(c2cc(N3CCN(C)C3=O)c3nc(C(=O)NCc4ccc(F)cc4)c(O)c(=O)n3c2)CC1]

This compound was prepared by adapting the procedure described in example 2.4.

¹H NMR (300 MHz, DMSO-d⁶) δ 11.95 (s, 1H), 9.25-9.10 (brs, 1H), 7.90 (d, J=2.4 Hz, 1H), 7.75 (d, J=2.4 Hz, 1H), 7.40 (dd, J=5.8, 8.8 Hz, 2H), 7.18 (t, J=8.8 Hz, 2H), 4.55 (d, J=6.3 Hz, 2H), 4.00 (t, J=8.0 Hz, 2H), 3.49 (t, J=8.0 Hz, 2H), 3.20-3.08 (m, 4H), 2.77 (s, 3H), 2.74-2.67 (m, 1H), 2.65-2.56 (m, 4H), 1.01 (d, J=6.5 Hz, 6H).

MS (ESI⁺) m/z 538 (M+1)

### Example 33

### Preparation of 3-Hydroxy-4-oxo-9-(1,3-propanesultam)-7-piperazin-1-yl-4H-pyrido[1,2-a]pyrimidine-2-carboxylic acid 4-fluoro-benzylamide

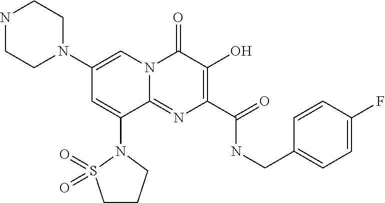 [SMILES: O=C(NCc1ccc(F)cc1)c1nc2c(N3CCCS3(=O)=O)cc(N3CCNCC3)cn2c(=O)c1O]

This compound was prepared by adapting the procedures described in examples 15 and example 24.

### Activity Assays

Compounds of the present invention were tested for biological activity using the assay techniques below:

### Inhibition of HIV Replication

HuT-78 cells are seeded into 96 well microtitre plates at 50,000 cells per 501 per well in RF-10 containing 2 μg/mL polybrene (RF-10/2). Compounds are prepared to 4× final concentration in RF-10/2, and 30 L added to cells. Virus (40 μL in RF-10/2 containing 1600 pfu) is added to each well or 40 L RF-10/2 for negative controls and for assaying compound cytotoxicity. After 24 hrs, an additional 90 μL of media or media containing 1× compound is added to each well. At 4 days post infection, 100 μL of media is removed from each well and replaced with 100 μl of fresh media with or without compound. Forty eight hours later supernatants are harvested and levels of extracellular p24 determined. Supernatants are diluted 1 in 10,000 and p24 levels assayed using the Vironostika p24 assay kit. EC₅₀ is calculated as the concentration required to inhibit HIV p24 production to 50% that of no drug controls.

In the tables below, this assay type is referred to as NL4-3.

### Luciferase Assay

Reporter Viruses:

Infectivity assays using reporter viruses derived from lentiviral vectors capable of a single round of infection were used to determine the activity (EC₅₀) of compounds. The DNA used to generate viruses for infection was the full-length HIV-1 genome which had been envelope-deleted. In addition, a reporter gene (the firefly luciferase gene from Photinus pyralis) was cloned into the nefregion of the HIV backbone for ease of assay readout. Viruses were generated via liposomal transfection of the lentiviral-derived DNA backbone together with a vesicular stomatitis virus glycoprotein (VSV-G) expression plasmid into 293T cells. Culture supernatants containing VSV-G pseudotyped virions were harvested 64 h post transfection, clarified by centrifugation to remove cell debris, and frozen at −70° C. until use.

### Assay Method:

293FT cells were plated out at 12000 cells per well in CellView 96-well cell culture plates (Invitrogen) 16 h prior to compound addition. Compounds were preincubated with cells for 4 h at 37° C. prior to the addition of virus sufficient to generate approximately 10000 Luciferase light units (as measured by the Victor Wallace luminometer) upon assaying using the Bright-Glo™ reagent (Promega) according to the manufacturer's instructions at 48 h post infection.

In the tables below, this assay is referred to as NLXLuc or HIV-Luc.

TABLE 1

| Example | Structure | Assay Type | Cell type | EC50 |
|---|---|---|---|---|
| 3.4 | 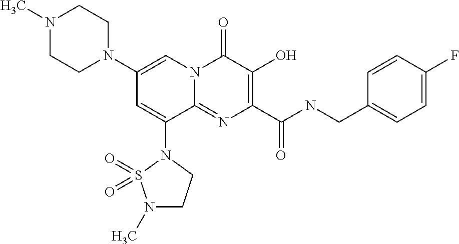 [SMILES: CN1CCN(c2cc(N3CCN(C)S3(=O)=O)c3nc(C(=O)NCc4ccc(F)cc4)c(O)c(=O)n3c2)CC1] | NLXLuc (WT) | 293FT | +++++ |
| 5.4 | 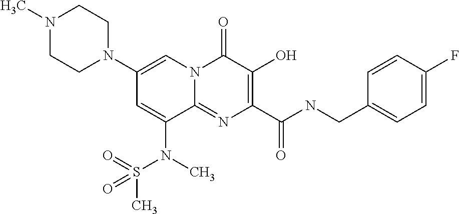 [SMILES: CN1CCN(c2cc(N(C)S(C)(=O)=O)c3nc(C(=O)NCc4ccc(F)cc4)c(O)c(=O)n3c2)CC1] | NLXLuc (WT) | 293FT | ++++ |
| 1.4 | 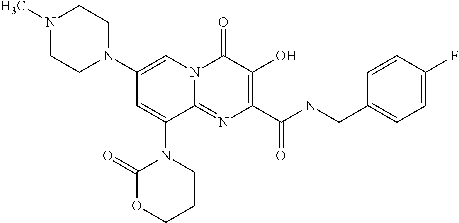 [SMILES: CN1CCN(c2cc(N3CCCOC3=O)c3nc(C(=O)NCc4ccc(F)cc4)c(O)c(=O)n3c2)CC1] | NLXLuc (WT) | 293FT | ++ |
| 2.4 | 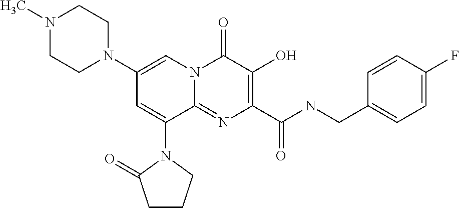 [SMILES: CN1CCN(c2cc(N3CCCC3=O)c3nc(C(=O)NCc4ccc(F)cc4)c(O)c(=O)n3c2)CC1] | NLXLuc (WT) | 293FT | ++ |
| 14.3 | 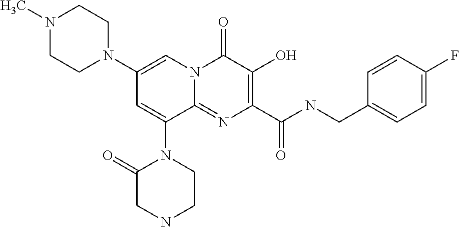 [SMILES: CN1CCN(c2cc(N3CCNCC3=O)c3nc(C(=O)NCc4ccc(F)cc4)c(O)c(=O)n3c2)CC1] | NLXLuc (WT) | 293FT | ++ |
| 17.4 | 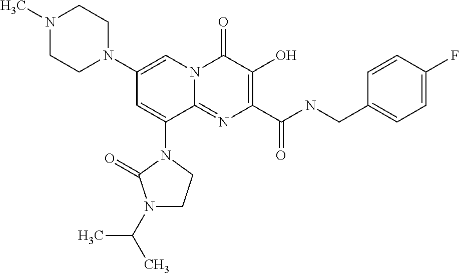 [SMILES: CC(C)N1CCN(c2cc(N3CCN(C)CC3)cn3c(=O)c(O)c(C(=O)NCc4ccc(F)cc4)nc23)C1=O] | NLXLuc (WT) | 293FT | +++ |
| 18.4 | 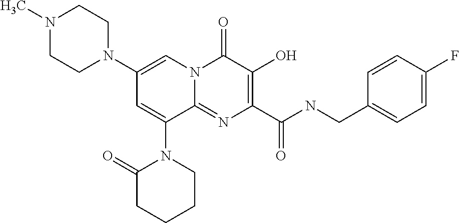 [SMILES: CN1CCN(c2cc(N3CCCCC3=O)c3nc(C(=O)NCc4ccc(F)cc4)c(O)c(=O)n3c2)CC1] | NLXLuc (WT) | 293FT | +++ |
| 20.4 | 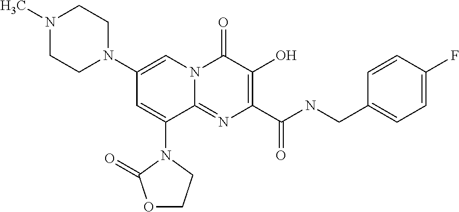 [SMILES: CN1CCN(c2cc(N3CCOC3=O)c3nc(C(=O)NCc4ccc(F)cc4)c(O)c(=O)n3c2)CC1] | NLXLuc (WT) | 293FT | +++ |
| comparative example | 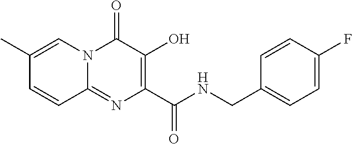 [SMILES: Cc1ccc2nc(C(=O)NCc3ccc(F)cc3)c(O)c(=O)n2c1] | NLXLuc (WT) | 293FT | + |

++++++ indicates value less than 1 nM
+++++ indicates value between 1 nM and 10 nM
++++ indicates value between 10 nM and 20 nM
+++ indicates value between 20 nM and 100 nM
++ indicates value between 100 nM and 1 μM
+ indicates value greater than 1 μM

TABLE 2

| Example | Structure | Assay Type | Cell type | EC50 |
|---|---|---|---|---|
| 24.4 | 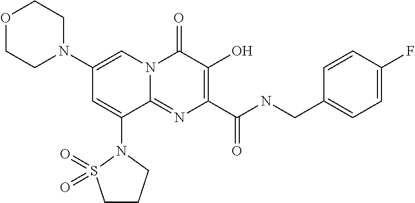 [SMILES: O=C(NCc1ccc(F)cc1)c1nc2c(N3CCCS3(=O)=O)cc(N3CCOCC3)cn2c(=O)c1O] | NL4-3 | HuT-78 | ++++++ |
| 28.3 | 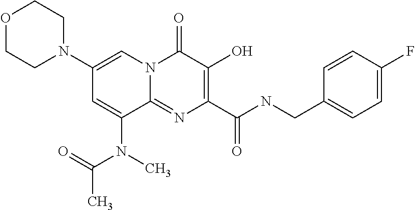 [SMILES: CC(=O)N(C)c1cc(N2CCOCC2)cn2c(=O)c(O)c(C(=O)NCc3ccc(F)cc3)nc12] | NL4-3 | HuT-78 | +++++ |
| 23.4 | 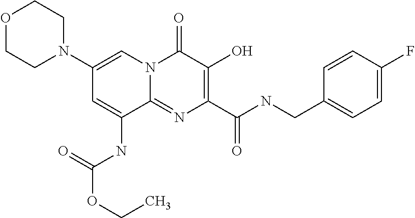 [SMILES: CCOC(=O)Nc1cc(N2CCOCC2)cn2c(=O)c(O)c(C(=O)NCc3ccc(F)cc3)nc12] | NL4-3 | HuT-78 | +++++ |
| 26.5 | 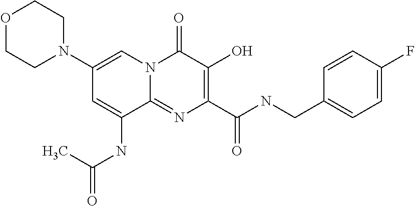 [SMILES: CC(=O)Nc1cc(N2CCOCC2)cn2c(=O)c(O)c(C(=O)NCc3ccc(F)cc3)nc12] | NL4-3 | HuT-78 | +++ |
| comparative example | 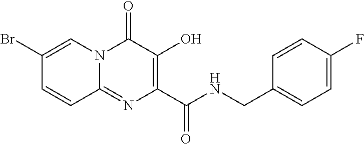 [SMILES: O=C(NCc1ccc(F)cc1)c1nc2ccc(Br)cn2c(=O)c1O] | NL4-3 | HuT-78 | ++ |
| comparative example | 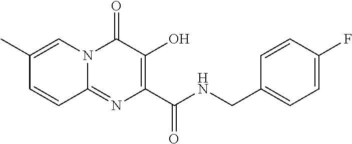 [SMILES: Cc1ccc2nc(C(=O)NCc3ccc(F)cc3)c(O)c(=O)n2c1] | NL4-3 | HuT-78 | ++ |

TABLE 3

| Example | Structure | Assay Type | Cell type | EC50 |
|---|---|---|---|---|
| 25 | 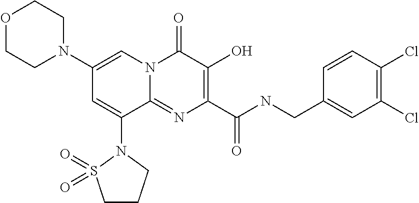 [SMILES: O=C(NCc1ccc(Cl)c(Cl)c1)c1nc2c(N3CCCS3(=O)=O)cc(N3CCOCC3)cn2c(=O)c1O] | HIV-Luc | 293T | ++++++ |
| 30 | 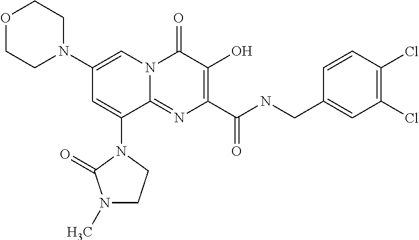 [SMILES: CN1CCN(c2cc(N3CCOCC3)cn3c(=O)c(O)c(C(=O)NCc4ccc(Cl)c(Cl)c4)nc23)C1=O] | NLXLuc (WT) | 293FT | +++ |
| comparative example | 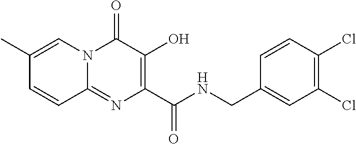 [SMILES: Cc1ccc2nc(C(=O)NCc3ccc(Cl)c(Cl)c3)c(O)c(=O)n2c1] | NLXLuc (WT) | 293FT | ++ |

TABLE 4

| Example | Structure | Assay Type | Cell type | EC50 |
|---|---|---|---|---|
| 25 | 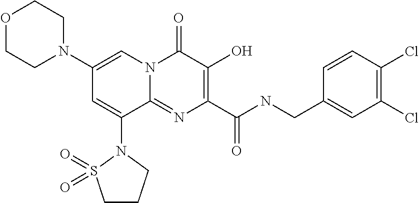 [SMILES: O=C(NCc1ccc(Cl)c(Cl)c1)c1nc2c(N3CCCS3(=O)=O)cc(N3CCOCC3)cn2c(=O)c1O] | NL4-3 | HuT-78 | ++++++ |
| 22 | 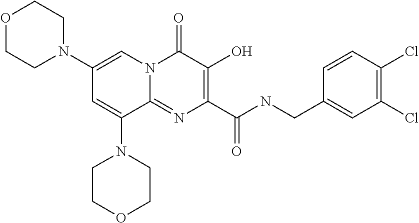 [SMILES: O=C(NCc1ccc(Cl)c(Cl)c1)c1nc2c(N3CCOCC3)cc(N3CCOCC3)cn2c(=O)c1O] | NL4-3 | HuT-78 | +++++ |
| comparative example | 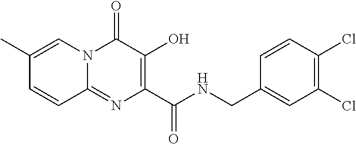 [SMILES: Cc1ccc2nc(C(=O)NCc3ccc(Cl)c(Cl)c3)c(O)c(=O)n2c1] | NL4-3 | HuT-78 | ++ |

As can be seen from Tables 1 to 4, the compounds of the present invention show surprisingly enhanced activity against the HIV virus when compared to compounds which have previously been exemplified.

TABLE 5

| Example | Structure | Assay Type | Cell type | EC50 |
|---|---|---|---|---|
| 24.4 | 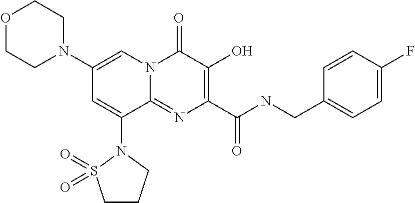 [SMILES: O=C(NCc1ccc(F)cc1)c1nc2c(N3CCCS3(=O)=O)cc(N3CCOCC3)cn2c(=O)c1O] | NLXLuc (WT) | 293FT | ++++ |
| 21.5 | 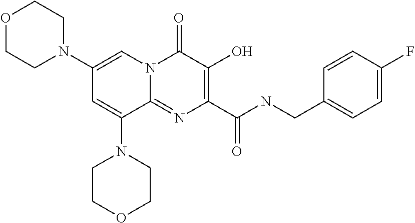 [SMILES: O=C(NCc1ccc(F)cc1)c1nc2c(N3CCOCC3)cc(N3CCOCC3)cn2c(=O)c1O] | NLXLuc (WT) | 293FT | ++ |
| 28.3 | 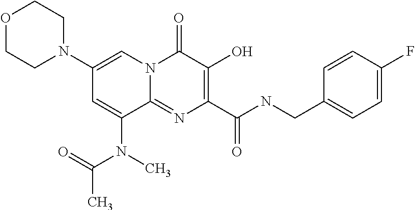 [SMILES: CC(=O)N(C)c1cc(N2CCOCC2)cn2c(=O)c(O)c(C(=O)NCc3ccc(F)cc3)nc12] | NLXLuc (WT) | 293FT | +++ |
| 29.2 | 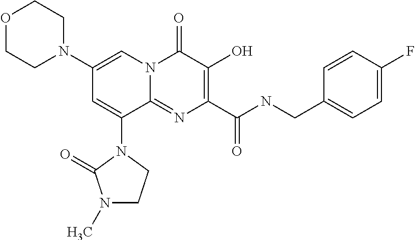 [SMILES: CN1CCN(c2cc(N3CCOCC3)cn3c(=O)c(O)c(C(=O)NCc4ccc(F)cc4)nc23)C1=O] | NLXLuc (WT) | 293FT | +++ |

TABLE 6

| Example | Structure | Assay Type | Cell type | EC50 |
|---|---|---|---|---|
| 4.4 | 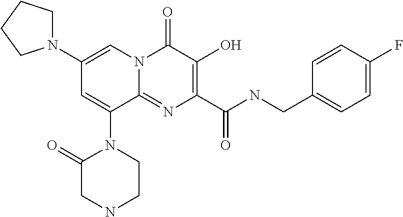 [SMILES: O=C(NCc1ccc(F)cc1)c1nc2c(N3CCNCC3=O)cc(N3CCCC3)cn2c(=O)c1O] | NLXLuc (WT) | 293FT | ++ |
| 6.3 | 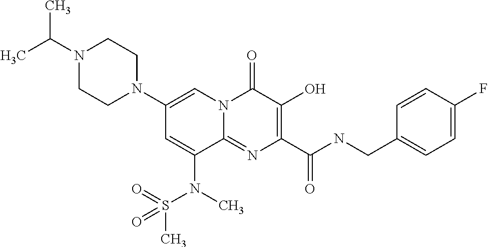 [SMILES: CC(C)N1CCN(c2cc(N(C)S(C)(=O)=O)c3nc(C(=O)NCc4ccc(F)cc4)c(O)c(=O)n3c2)CC1] | NLXLuc (WT) | 293FT | ++++ |
| 7.3 | 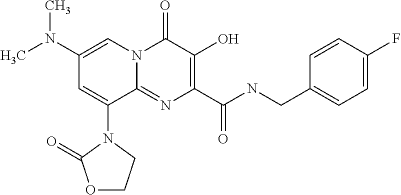 [SMILES: CN(C)c1cc(N2CCOC2=O)c2nc(C(=O)NCc3ccc(F)cc3)c(O)c(=O)n2c1] | NLXLuc (WT) | 293FT | ++ |
| 8.3 | 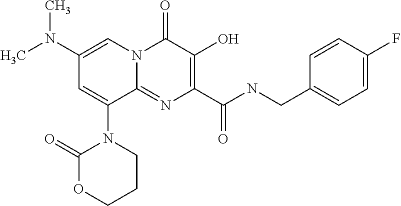 [SMILES: CN(C)c1cc(N2CCCOC2=O)c2nc(C(=O)NCc3ccc(F)cc3)c(O)c(=O)n2c1] | NLXLuc (WT) | 293FT | +++ |
| 9.3 | 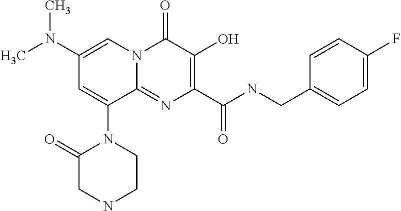 [SMILES: CN(C)c1cc(N2CCNCC2=O)c2nc(C(=O)NCc3ccc(F)cc3)c(O)c(=O)n2c1] | NLXLuc (WT) | 293FT | ++ |
| 10.3 | 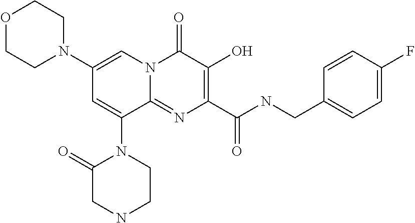 [SMILES: O=C(NCc1ccc(F)cc1)c1nc2c(N3CCNCC3=O)cc(N3CCOCC3)cn2c(=O)c1O] | NLXLuc (WT) | 293FT | ++ |
| 11.3 | 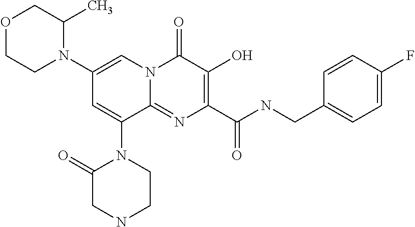 [SMILES: CC1COCCN1c1cc(N2CCNCC2=O)c2nc(C(=O)NCc3ccc(F)cc3)c(O)c(=O)n2c1] | NLXLuc (WT) | 293FT | ++ |
| 12.3 | 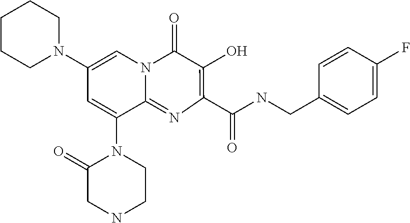 [SMILES: O=C(NCc1ccc(F)cc1)c1nc2c(N3CCNCC3=O)cc(N3CCCCC3)cn2c(=O)c1O] | NLXLuc (WT) | 293FT | +++ |
| 13.4 | 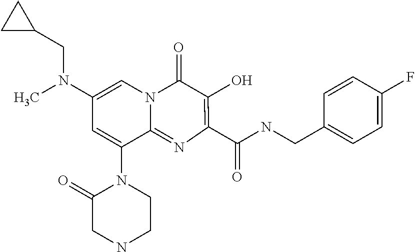 [SMILES: CN(CC1CC1)c1cc(N2CCNCC2=O)c2nc(C(=O)NCc3ccc(F)cc3)c(O)c(=O)n2c1] | NLXLuc (WT) | 293FT | +++ |
| 15.3 | 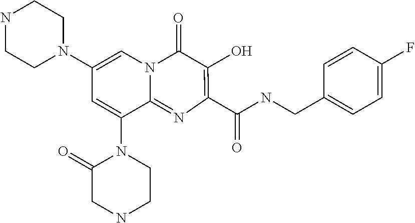 [SMILES: O=C(NCc1ccc(F)cc1)c1nc2c(N3CCNCC3=O)cc(N3CCNCC3)cn2c(=O)c1O] | NLXLuc (WT) | 293FT | ++ |
| 16.4 | 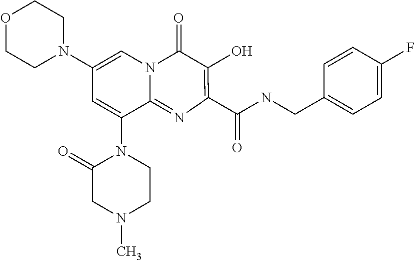 [SMILES: CN1CCN(c2cc(N3CCOCC3)cn3c(=O)c(O)c(C(=O)NCc4ccc(F)cc4)nc23)C(=O)C1] | NLXLuc (WT) | 293FT | +++ |
| 19.4 | 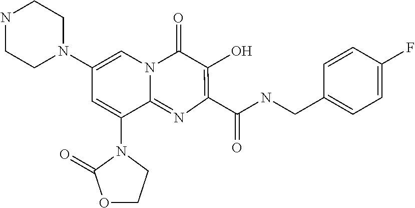 [SMILES: O=C(NCc1ccc(F)cc1)c1nc2c(N3CCOC3=O)cc(N3CCNCC3)cn2c(=O)c1O] | NLXLuc (WT) | 293FT | + |
| 31.3 | 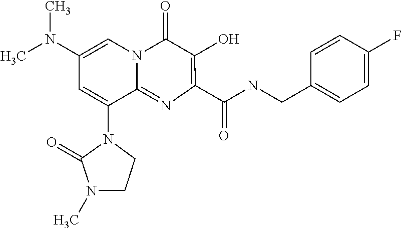 [SMILES: CN1CCN(c2cc(N(C)C)cn3c(=O)c(O)c(C(=O)NCc4ccc(F)cc4)nc23)C1=O] | NLXLuc (WT) | 293FT | ++ |
| 32.4 | 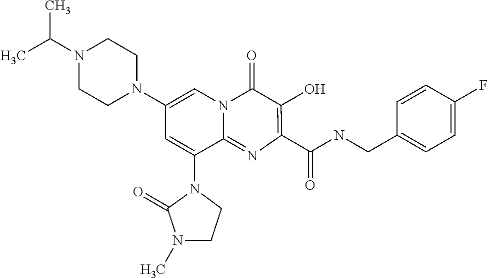 [SMILES: CC(C)N1CCN(c2cc(N3CCN(C)C3=O)c3nc(C(=O)NCc4ccc(F)cc4)c(O)c(=O)n3c2)CC1] | NLXLuc (WT) | 293FT | ++++ |
| 33 | 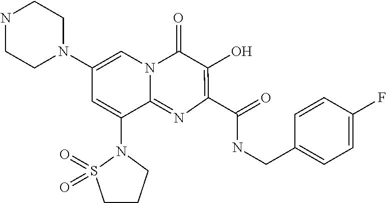 [SMILES: O=C(NCc1ccc(F)cc1)c1nc2c(N3CCCS3(=O)=O)cc(N3CCNCC3)cn2c(=O)c1O] | NLXLuc (WT) | 293FT | ++ |

### Evaluation of Bioavailability and Pharmacokinetic Profiles of the Compound of Example 3.4 in Male Sprague Dawley Rats Following Intravenous and Oral Administration

Method: For IV infusion over 10 minutes the compound of Example 3.4 was dissolved in 20% DMSO, 30% PEG400 and 50% Water. For oral (PO) dosing AVX62738 was dissolved in 0.5% benzyl alcohol and 0.4% Tween 80 and 99.1% 0.5% hydroxypropylmethylcellulose. Compounds were dosed IV and then after a one week washout, orally in the same rats. (Body weight: Rat 1: 254.7 g; Rat 2: 253.4 g). Ex-vivo plasma samples were assayed for AVX62738 using an LC-ms method of Lower limit of quantitation. (LLOQ)=2.5 ng/mL.

### Results:

TABLE 7
Plasma Concentrations of Compound 3.4 in Male Sprague Dawley
Rats following Intravenous and Oral Administration
Plasma Concentration (ng/mL)
Animal Number

| Sample | | | | | | |
|---|---|---|---|---|---|---|
| Collection | | | | | | |
| Time | AVX62738 (IV-5 mg/kg) | | | AVX62738 (PO-10 mg/kg) | | |
| Point (hr) | Rat 1 | Rat 2 | Mean | Rat 1 | Rat 2 | Mean |
| Pre-dose | BLQ | BLQ | NA | BLQ | BLQ | NA |
| 0.083 | 2713.49 | 3352.49 | 3032.99 | 42.37 | 28.06 | 35.21 |
| 0.25 | 1994.77 | 2133.03 | 2063.90 | 66.22 | 78.08 | 72.15 |
| 0.5 | 1211.86 | 1064.57 | 1138.21 | 35.66 | 91.60 | 63.63 |
| 1 | 547.66 | 506.38 | 527.02 | 20.94 | 58.11 | 39.52 |
| 2 | 122.82 | 103.78 | 113.30 | 13.83 | 14.31 | 14.07 |
| 4 | 19.34 | 18.73 | 19.03 | 5.62 | 4.96 | 5.29 |
| 6 | 5.44 | 6.97 | 6.21 | 2.62 | BLQ | 1.31 |
| 8 | 2.64 | 2.19 | 2.41 | 148.29* | BLQ | NA |
| 24 | BLQ | BLQ | NA | 4.66* | BLQ | NA |

*The concentrations of these timepoints were not kept for calculation.
Lower limit of quantitation. (LLOQ) = 2.5 ng/mL
BLQ: Below limit of quantitation
SD: Standard deviation
NA: Not applicable, or failed to collect samples

TABLE 8
Selected Pharmacokinetics Parameters of Compound 3.4 in Male Sprague
Dawley Rats following Intravenous and Oral Administration

| | | | | | | | | |
|---|---|---|---|---|---|---|---|---|
| AUC₍₀₋ₜ₎ | AUC_{(0-∞)} | MRT_{(0-∞)} | t_{1/2z} | Tₘₐₓ | Vz | CLz | Cₘₐₓ | F |
| μg/L*hr | μg/L*hr | hr | hr | hr | L/kg | L/hr/kg | μg/L | % |

IV (5 mg/kg)

| | | | | | | | | |
|---|---|---|---|---|---|---|---|---|
| 701 | 1856.72 | 1862.02 | 0.68 | 1.39 | 0.08 | 5.39 | 2.69 | 2713.49 |
| 702 | 1852.06 | 1856.14 | 0.63 | 1.29 | 0.08 | 5.02 | 2.69 | 3352.49 |
| Mean | 1854.39 | 1859.08 | 0.66 | 1.34 | 0.08 | 5.21 | 2.69 | 3032.99 |

PO (10 mg/kg)

| | | | | | | | | | |
|---|---|---|---|---|---|---|---|---|---|
| 801 | 82.79 | 89.04 | 2.03 | 1.65 | 0.25 | NA | NA | 66.22 | 2.39 |
| 802 | 124.14 | 130.06 | 1.22 | 0.83 | 0.50 | NA | NA | 91.60 | 3.50 |
| Mean | 103.47 | 109.55 | 1.63 | 1.24 | 0.38 | NA | NA | 78.91 | 2.95 |

TABLE 9
Clinical Observation
Clinical Observation

Following intravenous administration, no obvious abnormalities were
observed..
Following oral administration, no obvious abnormalities were observed.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

1. A compound of Formula I or a pharmaceutically acceptable derivative, salt or prodrug thereof wherein: 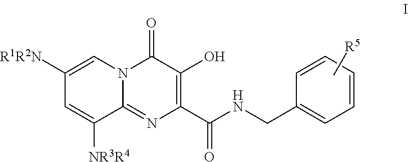
R¹ and R² are each independently selected from the group consisting of hydrogen, C₁₋₄alkyl, C₁₋₄alkylC₃₋₆cycloalkyl, C(O)C₁₋₄alkyl, CO₂C₁₋₄alkyl, —C(O)C(O)NR⁶R⁷, SO₂C₁₋₄alkyl, SO₂NR⁶R⁷; or R¹ and R² taken together with the attached nitrogen form a 4-7 membered heterocyclic ring which contains zero to two additional heteroatoms selected from N, O or S where S can be at the S, S(O) or S(O)₂ oxidation state and wherein said heterocyclic ring is optionally substituted at the carbon or nitrogen atoms with one or more substituents selected from C₁₋₄alkyl, C₃₋₆cycloalkyl, halo, aryl, C(O)C₁₋₄alkyl, SO₂C₁₋₄alkyl, SO₂H, CO₂H, CO₂C₁₋₄alkyl, NR⁶R⁷, C₁₋₄alkylNR⁶R⁷; and further wherein one of the carbon atoms in the heterocyclic ring is optionally a carbonyl carbon;
wherein R⁶ and R⁷ are independently selected from the group consisting of hydrogen and C₁₋₄alkyl, and C₃₋₆cycloalkyl; or R⁶ and R⁷ taken together with the attached nitrogen form a 4-7 membered heterocyclic ring which contains zero to two additional heteroatoms selected from N and O;
R³ and R⁴ are each independently selected from the group consisting of hydrogen, C₁₋₄alkyl, C₁₋₄alkylC₃₋₆cycloalkyl, C(O)C₁₋₄alkyl, CO₂C₁₋₄alkyl, —C(O)C(O)NR⁸R⁹, SO₂C₁₋₄alkyl, SO₂NR⁸R⁹; or R³ and R⁴ taken together with the attached nitrogen form a 4-7 membered heterocyclic ring which contains zero to two additional heteroatoms selected from N, O or S where S can be at the S, S(O) or S(O)₂ oxidation state and wherein said heterocyclic ring is optionally substituted at the carbon or nitrogen atoms with one or more substituents selected from C₁₋₄alkyl, C₃₋₆cycloalkyl, halo, aryl, C(O)C₁₋₄alkyl, SO₂C₁₋₄alkyl, SO₂H, CO₂H, CO₂C₁₋₄alkyl, NR⁸R⁹; C₁₋₄alkylNR⁸R⁹; and further wherein one of the carbon atoms in the heterocyclic ring is optionally a carbonyl carbon;
wherein R⁸ and R⁹ are each independently selected from the group consisting of hydrogen, C₁₋₄alkyl and C₃₋₆cycloalkyl; or R⁸ and R⁹ taken together with the attached nitrogen form a 4-7 membered heterocyclic ring which contains zero to two additional heteroatoms selected from N and O;
R⁵ is 0-3 substituents each of which is independently selected from the group consisting of halo, C₁₋₁₀alkyl, C₂₋₁₀alkenyl, —O—C₁₋₁₀alkyl, C(O)C₁₋₄alkyl CO₂H, CO₂C₁₋₄alkyl, CN, NH₂, NO₂, CF₃, aryl, heteroaryl, alkylaryl, alkylheteroaryl, —O-alkylaryl.

2. A compound according to claim 1 wherein R³ and R⁴ taken together with the attached nitrogen form a 4-7 membered heterocyclic ring which contains zero to two additional heteroatoms selected from N, O or S where S can be at the S, S(O) or S(O)₂ oxidation state and wherein said heterocyclic ring is optionally substituted at the carbon or nitrogen atoms with one or more substituents selected from C₁₋₄alkyl.

3. A compound according to claim 1, wherein R³ and R⁴ taken together with the attached nitrogen forms a 4-7 membered heterocyclic ring which contains at least one additional sulfur heteroatom in the S(O)₂ oxidation state adjacent to the attached nitrogen, and wherein the ring contains one additional nitrogen atom, wherein the additional nitrogen atom is optionally substituted with C₁₋₄alkyl.

4. A compound according to claim 3, wherein the additional nitrogen is substituted with methyl.

5. A compound according to claim 1, wherein NR³R⁴ is selected from the group consisting of:

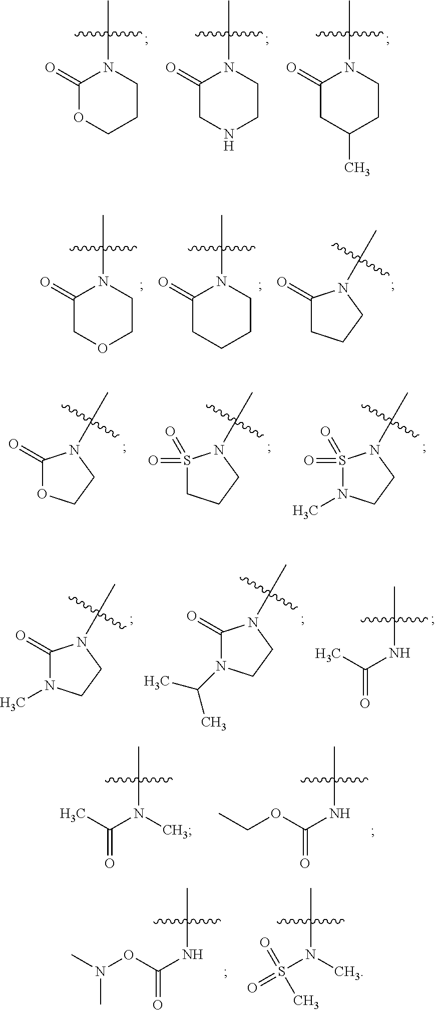

6. A compound of Formula I or a pharmaceutically acceptable derivative, salt or prodrug thereof: 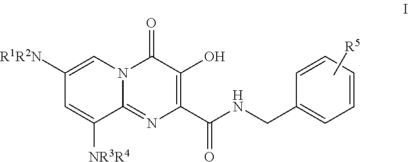
wherein R¹ and R² are each independently selected from the group consisting of hydrogen, C₁₋₄alkyl, C₁₋₄alkylC₃₋₆cycloalkyl, C(O)C₁₋₄alkyl, CO₂C₁₋₄alkyl, —C(O)C(O)NR⁶R⁷, SO₂C₁₋₄alkyl, SO₂NR⁶R⁷; or R¹ and R² taken together with the attached nitrogen form a 4-7 membered heterocyclic ring which contains zero to two additional heteroatoms selected from N, O or S where S can be at the S, S(O) or S(O)₂ oxidation state and wherein said heterocyclic ring is optionally substituted at the carbon or nitrogen atoms with one or more substituents selected from C₁₋₄alkyl, C₃₋₆cycloalkyl, halo, aryl, C(O)C₁₋₄alkyl, SO₂C₁₋₄alkyl, SO₂H, CO₂H, CO₂C₁₋₄alkyl, NR⁶R⁷, C₁₋₄alkylNR⁶R⁷; and further wherein one of the carbon atoms in the heterocyclic ring is optionally a carbonyl carbon;
wherein R⁶ and R⁷ are independently selected from the group consisting of hydrogen and C₁₋₄alkyl, and C₃₋₆cycloalkyl; or R⁶ and R⁷ taken together with the attached nitrogen form a 4-7 membered heterocyclic ring which contains zero to two additional heteroatoms selected from N and O;
wherein R³ is C₁₋₄alkyl and R⁴ is SO₂C₁₋₄alkyl;
or wherein NR³R⁴ forms a cyclic sulphonamide of the formula II: 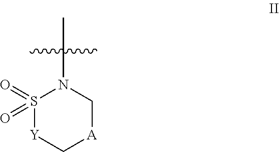
wherein Y is selected from the group consisting of a bond, CH₂, NH and NC₁₋₄alkyl; and A is a bond or CH₂;
wherein R⁵ is 0-3 substituents each of which is independently selected from the group consisting of halo, C₁₋₁₀alkyl, C₂₋₁₀alkenyl, —O—C₁₋₁₀alkyl, C(O)C₁₋₄alkyl CO₂H, CO₂C₁₋₄alkyl, CN, NH₂, NO₂, CF₃, aryl, heteroaryl, alkylaryl, alkylheteroaryl, —O-alkylaryl.

7. A compound according to claim 1, wherein R¹ and R² taken together with the attached nitrogen form a 4-7 membered heterocyclic ring which contains zero to two additional heteroatoms selected from N or O, wherein said heterocyclic ring is optionally substituted at the carbon or nitrogen atoms with one or more C₁₋₄alkyl substituents.

8. A compound according to claim 1, wherein R¹ and R² taken together with the attached nitrogen form morpholine.

9. A compound according to claim 1, wherein R¹ and R² are taken together with the attached nitrogen form piperazine

10. A compound according to claim 1, wherein R¹ and R² are taken together with the attached nitrogen form N-methyl piperazine.

11. A compound according to claim 1, wherein NR¹R² is selected from the group consisting of:

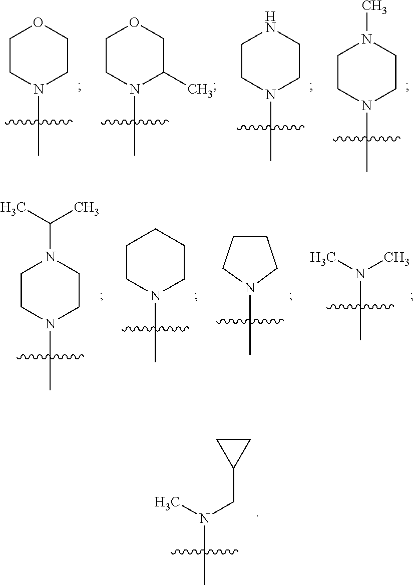

12. A compound according to claim 1, wherein R⁵ is 1-2 substituents each independently selected from halo.

13. A compound according to claim 1, wherein R⁵ is 1-2 substituents each independently selected from Cl or F.

14. A compound according to claim 1 wherein R⁵ is a fluorine substituent at the 4-position of the phenyl ring.

15. A compound according to claim 1 wherein R⁵ is two chlorine substituents at the 3 and 4-position of the phenyl ring.

16. A compound selected from the group consisting of:

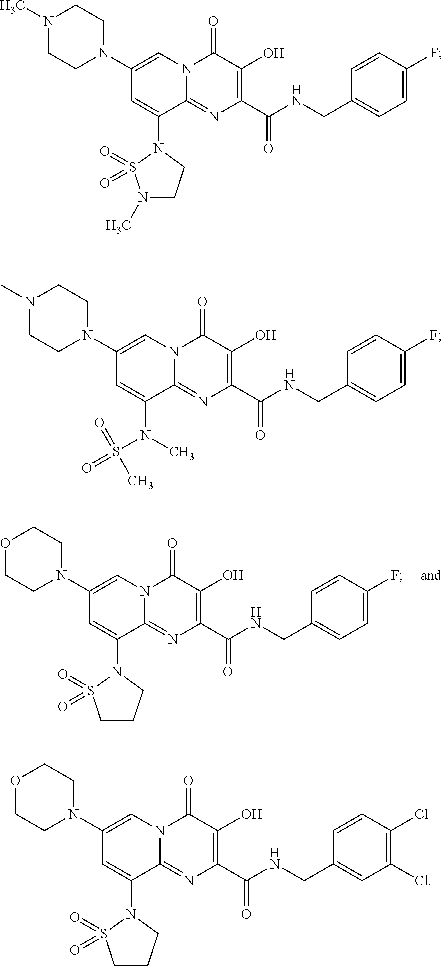 [SMILES: CN1CCN(c2cc(N3CCN(C)S3(=O)=O)c3nc(C(=O)NCc4ccc(F)cc4)c(O)c(=O)n3c2)CC1 ; CN1CCN(c2cc(N(C)S(C)(=O)=O)c3nc(C(=O)NCc4ccc(F)cc4)c(O)c(=O)n3c2)CC1 ; O=C(NCc1ccc(F)cc1)c1nc2c(N3CCCS3(=O)=O)cc(N3CCOCC3)cn2c(=O)c1O ; "and" ; O=C(NCc1ccc(Cl)c(Cl)c1)c1nc2c(N3CCCS3(=O)=O)cc(N3CCOCC3)cn2c(=O)c1O]

17. A method of treatment or prophylaxis of a viral infection in a subject comprising administering to said subject an effective amount of a compound of claim 16 or a pharmaceutically acceptable derivative, salt or prodrug thereof.

18. (canceled)

19. A method according to claim 17, wherein the viral infection is a HIV or SIV infection.

20. A pharmaceutical composition comprising a compound according to claim 1 and a pharmaceutically acceptable carrier, diluent or excipient.
